(19) United States
(12) Patent Application Publication
(10) Pub. No.: US 2021/0236557 A1
(43) Pub. Date: Aug. 5, 2021

(54) IMMATURE DENTAL PULP STEM CELLS AND METHODS OF USE TO TREAT BONE MARROW FAILURE

(71) Applicants: AVITA INTERNATONAL LTD., Tortola (VG); FUNDAÇÃO BUTANTAN, São Paulo, SP (BR)

(72) Inventors: Irina Kerkis, São Paulo (BR); Cristiane Valverde Wenceslau, São Paulo, SP (BR); Vivian Fonseca Gonzaga, São Paulo, SP (BR)

(21) Appl. No.: 17/049,956

(22) PCT Filed: Apr. 30, 2019
(86) PCT No.: PCT/IB2019/053513

§ 371 Date: Oct. 22, 2020

Related U.S. Application Data
(60) Provisional application No. 62/664,894, filed on Apr. 30, 2018.

(51) Int. Cl. A61K 35/28; C07K 14/705; C07K 14/78; C12N 5/0775

(52) U.S. Cl. CPC A61K 35/28; C12N 5/0664; C07K 14/78; C07K 14/70596

(57) ABSTRACT

The present invention relates to the field of adult stem cells of embryonic neural crest origin, in particular, multifunctional immature dental pulp stem cells (IDPSCs). The invention discloses useful compositions and methods for the prevention or treatment of a wide range of diseases that lead to or derived from bone marrow failure (BMF), Acute Radiation Syndromes, or chemical injury through the use of isolated IDPSC population with minimal in vitro manipulation.

### CROSS-REFERENCE TO RELATED APPLICATIONS

This application claims priority to and the benefit of U.S. Provisional patent Applications No. 62/664,894 filed on Apr. 30, 2018. The content of which is incorporated herein by reference in its entirety.

### INCORPORATION BY REFERENCE IN ITS ENTIRETY

This application cites U.S. Pat. No. 9,790,468, filed Mar. 14, 2014. The content of which is incorporated herein by reference in its entirety.

### FIELD OF INVENTION

The present invention relates to adult stem cells of embryonic neural crest origin. More specifically, this disclosure relates to multifunctional immature dental pulp stem cells (IDPSCs), pharmaceutical compositions comprising IDPSCs, and methods of treating conditions associated with bone marrow failure (BMF).

### BACKGROUND OF INVENTION

Red bone marrow (BM) is a gluey, complex, and heterogeneous tissue found in the medullary cavity of long bones and spongy bones cavities of the body. It is anatomically made up of the stromal cells (fibroblasts, adventitial reticular cells, adipocytes, and others) responsible for the tissue structure and the parenchymal cells (hematopoietic cells—blood-producing cells). To fabricate these blood-producing cells, BM contains a pool of hematopoietic stem cells (HSCs), which are self-renewing cells and differentiate into red (erythrocytes) and white (leukocytes) blood cells and generate megakaryocytes and those produce platelets (PLT). Only mature hematopoietic cells enter the bloodstream. With age, red BM tends to be substituted with yellow BM, which is mostly made up of fat cells.

BM stroma is a key element of hematopoiesis that provides the structural and physiological support for blood cells production. It also consists of a heterogeneous population of different cell types among which is a rare population of non-hematopoietic skeletal progenitor cells named Bone Marrow Stromal Cells (BMSC). Red BM (hematopoietic marrow) and stroma are crucial components of the hematopoietic microenvironment as they interact and produce together—or individually—humoral growth and/or inhibitory factors necessary to maintain normal hematopoiesis, which is essential for life and human health.

BM can be susceptible to two types of failure syndromes: inherited or acquired. The inherited bone marrow failure (BMF) syndromes are a group of disorders characterized by BMF in association with one or more somatic abnormality. It is usually diagnosed in childhood and passed down from parent to child through the association with some genetic abnormality, which may cause the aplastic anemia (AA) and cancer predisposition. Young people and adults may develop the acquired BMF, which can be caused by different extrinsic and intrinsic factors including chemicals, irradiation, chemotherapy treatments, and immune system harms.

Among BMF diseases, the acquired AA is more common. AA was first described in 1888 by Paul Erich merely as an “empty” BM with replacement by fat cells and now is defined by decreased hematopoietic precursors in the BM, resulting in BM hypoplasia, peripheral blood (PB) pancytopenia, and precocious fat replacement. AA pathophysiology may be immune-mediated; oligoclonal expanded T-cells destroy autologous stem cells by inducing apoptosis; transcription factor T-bet (T-box expressed in T cells) is up-regulated in T cells from patients with AA and decreased levels of T regulatory cells is also observed. A small proportion of acquired cases share pathophysiology with the inherited disease-shortened telomeres.

The treatment of acquired AA depends on the patient's age, health, and the severity of the disease. To treat moderate cases of acquired AA typically require blood transfusions and supportive care with an antibiotic. However, many moderate cases may progress to severe AA (SAA). Therefore, to treat acquired SAA, HSCs transplant from matched sibling donor is a matter of choice, which in some cases is satisfactorily effective. It may be used in combination with immunosuppressive (IS) therapies. However, most patients have no access to immediate HSCs transplant due to the lack of a matched sibling donor.

Nevertheless, the success of HSCs transplant is limited due to late complications, such as graft rejection and relapse due to resurgent autoimmune attack, and more often due to development of Graft-versus-host disease (GVHD), whereas lack of response, relapse, and clonal evolution limit the success of immune-suppression drugs. Thus, there is a need for stem cells that self-renew, can differentiate into multi-lineage cells, and do not lead to late complications.

### SUMMARY OF THE INVENTION

The present invention provides useful compositions and methods of treating or preventing bone marrow failure.

In certain aspects, the method typically involves administering to a subject a composition comprising a population of isolated immature dental pulp stem cells (IDPSCs) in an amount sufficient to treat or prevent bone marrow failure in the subject.

In an exemplary embodiment, the method of treating or preventing bone marrow failure comprises administering IDPSCs capable of migrating to the bone marrow of the subject.

In another exemplary embodiment, the method treats or prevents bone marrow failure caused by radiation exposure. In a particular implementation, the bone marrow failure is caused by a radiation exposure at a dose of 1-30 Gy. In another particular implementation, the composition is administered between 24 and 60 hours after the radiation exposure.

In one embodiment, the IDPSCs are in an amount sufficient to increases fibroblast precursors in the bone marrow of the subject.

In another embodiment, the IDPSCs are in an amount sufficient to increase erythrocyte (red blood cell) count in the bone marrow of the subject.

In a further embodiment, the IDPSCs are in an amount sufficient to increase hematocrit level in the bone marrow of the subject.

In one aspect, the IDPSCs are in an amount sufficient to increase leukocyte (white blood cell) count in the bone marrow of the subject.

In another aspect, the IDPSCs are in an amount sufficient to increase megakaryocytes, Sca-1-positive cells, Nestin-positive cells, fibronectin-positive cells, CD44-positive cells, or a combination thereof in the bone marrow of the subject.

In a further aspect, the IDPSCs are in an amount sufficient to increase bone marrow cellularity in the subject.

In one implementation, the IDPSCs are in an amount sufficient to increase extracellular matrix remodeling in the bone marrow of the subject.

In another implementation, the IDPSCs are in an amount sufficient to reduce bone marrow cell apoptosis in the subject.

In a further implementation, the IDPSCs are in an amount sufficient to reduce serum levels of TNF-α and/or IL-17A in the subject.

In one embodiment, the IDPSCs are in an amount sufficient to increase serum levels of IL-4 and/or IL-10 in the subject.

In another embodiment, the IDPSCs are in an amount sufficient to improve hematopoietic recovery for at least 6 months.

In one aspect, the method comprises administering the composition at least twice. In a particular embodiment, the consecutive administrations have an interval of between 10 and 20 days.

In other aspects, the present invention provides a composition for treating or preventing bone marrow failure. Typically, the composition comprises a population of isolated immature dental pulp stem cells (IDPSCs) prepared by obtaining a dental pulp (DP); establishing an explant culture by placing the DP onto a plastic surface in a culture medium and allowing IDPSCs to migrate out of the DP; passaging the IDPSCs to expand the explant culture; mechanically transferring the DP onto a different plastic surface in the culture medium; and collecting the IDPSCs that adhere to the plastic surface from the explant culture, the population of IDPSCs comprising IDPSCs. In particular embodiments, the IDPSCs are obtained after five transfers and four or five passages.

In one implementation of the method or composition, at least 80% of the IDPSCs express CD117 (c-kit) and/or Fibronectin.

In another implementation, 96%-99% of the IDPSCs are positive for one or more of the following markers: CD105, CD73, CD44, CD117, Nestin, Vimentin, and Fibronectin.

In a particular implementation, 96%-99% of the IDPSCs are negative for one or more of the following markers: CD45, CD34-negative, and HLA DR.

In certain embodiment, the composition further includes bone-marrow stem-cells, hematopoietic stem cells, or both.

In certain exemplary embodiments, the subject is human, and/or the IDPSCs are allogeneic.

Exemplary compositions that are useful comprise 5×10⁶-1×10⁹ IDPSCs.

### BRIEF DESCRIPTION OF THE DRAWINGS

FIG. 1 depicts embryonic development ontology tree (adapted from http://discovery.lifemapsc.com/) demonstrating difference in embryonic origin observed in MSC and MSC-like cells. Early embryonic tissues originate from the ectoderm, mesoderm, endoderm (not shown) and extraembryonic mesoderm. Both bone marrow and adipose tissue MSC originate from mesoderm. Dental pulp MSC-like cells originate from the ectoderm.

FIG. 2 depicts a non-limiting scheme of treating bone marrow failure using IDPSC therapy. The chronological order: Pre-irradiation (D0) followed by irradiation, the subjects receive three IDPSC administrations: 48 hours after irradiation (D2), 15 days after the first administration (D17), and 15 days after the second administration (D32). Euthanasia was performed 30 days after the third administration (D62). Blood samples were collected on D0, 2, 17, 32, and 62, and the spleen was collected on D62.

FIGS. 3A-3E show positive immunostaining (FITC) for CD90, Fibronectin, Nestin, Vimentin, and CD44 expression in undifferentiated IDPSCs. Nucleus is stained with DAPI. Scale bars=5 μm. FIG. 3F quantifies percent IDPSCs expressing indicated MSC markers using flow cytometry.

FIGS. 4A-4C compare the body mass between IDPSC treatment and non-treatment groups. Body mass was significantly increased on D32 (*, P<0.05) and D62 (***, P<0.001). Asterisks indicate statistical significance (ANOVA).

FIGS. 5A-5B compare erythrocyte (red blood cell) counts between IDPSC treatment and non-treatment groups. The IDPSC treatment group displays a significant decrease on D2 (**, P<0.01) and D17 (***, P<0.001), a significant recovery (##, P<0.01) on D32, and maintaining normal erythrocyte counts (>11×10⁶/μL) up to D62. The IDPSC non-treatment group displays consecutive drops in erythrocyte counts up to D62. Before irradiation (D0), statistically significant difference between the groups was not detected.

FIGS. 6A-6B compare hematocrit levels (HTC %) between IDPSC treatment and non-treatment groups. The IDPSC treatment group displays a significant decrease on D2 (**, P<0.01) and D17 (***, P<0.001), a significant recovery (##, P<0.01) on D32, and maintaining normal hematocrit levels (>50%) up to D62. The IDPSC non-treatment group displays a significant reduction on D2 (**, P<0.01), the reduction peaking on D17 (***, P<0.001), and the reduction last until D62 (**, P<0.01). Asterisk indicates statistical significance (ANOVA). Before irradiation (D0), statistically significant difference between the groups was not detected.

FIGS. 7A-7C compare leukocyte (white blood cell) counts (×10³/μL) between IDPSC treatment and non-treatment groups. The IDPSC treatment group displays a significant reduction of the absolute values of leukocytes on D2 (***), D17, (***), D32 (**), and D62 (**), and a significant recovery (###) on D32. The IDPSC non-treatment group displays a significant reduction after irradiation (***), which was maintained until D62 (***). Asterisk indicates statistical significance (ANOVA), P<0.01 (**), P<0.001 (***), and P<0.001 (###). Before irradiation (D0), statistically significant difference between the groups was not detected.

FIGS. 8A-8D compare microphotography of BM sections stained with hematoxylin and eosin at a magnification of 40× or 200×. Without irradiation, spinal canals are filled with hematopoietic elements in BM. Irradiation causes a decrease in cellularity, a greater region of the spinal stromal (indicated by the arrows), and infiltrated red blood cells (FIG. 8D) (indicated by the larger asterisk in red) in BM of the irradiated group (48 hours after irradiation).

FIGS. 9A-9F compare microphotography of BM sections stained with hematoxylin and eosin at magnification of 40× or 200×. The BM of IDPSC treatment group displays spinal canal filled with medullary elements (FIGS. 9A-9B). The BM of IDPSCs non-treatment group displays medullary hypoplasia due to decreased cellularity and fat replacement (FIGS. 9C-9D), and BM aplasia with an absence of cells (FIGS. 9E-9F). Medullar stroma is indicated by the shorter, red arrow, fat cells are indicated by longer, black arrows, and infiltrated red blood cells are indicated by the asterisk.

FIGS. 10A-10F compare microphotography of the smears of BM at a magnification of 200×, 400×, or 1000×. Hematopoietic elements, hematopoietic precursors (FIGS. 10A-10B), and megakaryocytes (FIG. 10C) of IDPSC treatment group, are indicated by red, shorter arrows. FIGS. 10D-10F depict BM of IDPSC non-treatment group. Notice a decrease of hematopoietic elements. FIGS. 10E-10F depict adipose cells that replaced the hematopoietic elements (indicated by black, longer arrows).

FIGS. 11A-11G depict microphotography of the colony-forming unit assay (CFU-F) derived from the medullary flushing of IDPSC treated animals. FIG. 11A depicts 6-well culture plate after 21 days of plating stained with violet crystal. FIGS. 11B-11G depict cell colonies.

FIGS. 12A-12G depict microphotography of the colony-forming unit assay (CFU-F) derived from the medullary flushing of IDPSC non-treated animals.

FIG. 13. Compare the number of CFU-F in groups that are non-irradiated, irradiated without IDPSC treatment, and IDPSC treatment 48 hours after irradiation. Colony forming units were significantly reduced 48 hours after irradiation (***). The reduction was not recovered in the untreated group (***). IDPSC treatment significantly increased the efficiency of generating CFU (##). Asterisk indicates statistical significance (ANOVA) in the reduction of CFU levels between the experimental groups P<0.001 (***) and P<0.01. (##) indicates statistical significance in the increase of the number of CFUs.

FIGS. 14A-14D depict representative histograms from flow cytometric analysis of p53 phenotype in bone marrow analyzed in: control (FIG. 14A), IDPSC treatment 48 hours after irradiation (FIG. 14B), and irradiation without treatment with IDPSCs (FIG. 14C). FIG. 14D depicts the percentage of p53-immunopositive cells. There is a significant increase (*) of p53-positive cells 48 hours after irradiation in relation to the control. There is a decrease of p53-positive cells in both experimental groups, and the group treated with IDPSCs has a lower mean of p53-positive cells than the untreated group. Asterisks indicate statistical significance (ANOVA) between 48 hours after irradiation and control, P<0.05 (*). Graphs are generated by GraphPad Prism 5 software (GraphPad; PRISM, 2007). FIGS. 14E-14G1 depict Immunohistochemistry anti-p53 antibody (brown color). Treatment with hIDPSC promoted greater protection of affected BM cells after irradiation.

FIGS. 15A-15D depict representative histograms from flow cytometric analysis of Fibronectin phenotype in bone marrow analyzed in: control (FIG. 15A), IDPSC treatment 48 hours after irradiation (FIG. 15B), and irradiation without treatment with IDPSCs (FIG. 15C). FIG. 15D depicts the percentage of fibronectin-immunopositive cells. There is a significant decrease in fibronectin-positive cells (*) 48 hours after irradiation in relation to the control. There is an increase in both experimental groups, with the IDPSC treated being greater than in untreated. Asterisks indicate statistical significance (ANOVA) between irradiation and control, P<0.05 (*). Graphs are generated by GraphPad Prism 5 software (GraphPad; PRISM, 2007).

FIGS. 16A-16D depict microphotography of the immunocytofluorescence (FIGS. 16A-16B), immunocytochemistry (FIGS. 16C-16D), and immunohistochemistry (FIGS. 16E-16F) assays of the intermediate filament fibronectin in BM aspirate of the IDPSC-treated or untreated group. Fibronectin was detected in the cytoplasm of the treated groups (FIGS. 16A, 16C, and 16E) and untreated groups (FIGS. 16B, 16D, and 16F) in BM (FIGS. 16A-16B), the medullary aspirate cells cultured in vitro (FIGS. 16C-16D), and the extracellular matrix of the BM (FIGS. 16E-16F). White arrow shows FITC labeled cells with fibroblastoid morphology (FIG. 16C) from treated group. Notice a lack of labeled cells in untreated group (FIG. 16D).

FIGS. 17A-17D depict representative histograms from flow cytometric analysis of Nestin phenotype in bone marrow. Control (FIG. 17A), IDPSC treatment 48 hours after irradiation (FIG. 17B), and irradiation without treatment with IDPSCs (FIG. 17C). FIG. 17D depicts the percentage of fibronectin-immunopositive cells. There is a significant decrease of Nestin-positive cells (*) 48 hours after irradiation and in the group untreated with IDPSCs (**) in relation to the control. There is an increase in the group treated with IDPSCs (#) in relation to irradiation. Asterisks indicate statistical significance (ANOVA) between the groups, P<0.05 (*) and P<0.01 (**). # indicates statistical significance in the increase of expression of Nestin, P<0.05 (#).

FIGS. 18A-18F depict immunostaining for Nestin observed in treated (FIGS. 18A, 18C, and 18E) and untreated (FIGS. 18B, 18D, and 18F) groups. Positive immunostainings for Nestin were observed in the cytoplasm of BM (FIGS. 18A-18B), the medullary aspirate cells cultured in vitro (FIGS. 18C-18D), and the extracellular matrix of the BM (FIGS. 18E-18F).

FIGS. 19A-19D depict representative histograms from flow cytometric analysis of CD105 phenotype in bone marrow. Control (FIG. 19A), IDPSC treatment 48 hours after irradiation (FIG. 19B), and irradiation without treatment with IDPSCs (FIG. 19C). FIG. 19D depicts the percentage of CD105-immunopositive cells. There is a tendency to an increase of CD105-positive cells in the group treated with IDPSCs (#) in relation to the irradiated group. No statistical significance between the groups was observed.

FIGS. 20A-20D depict representative histograms from flow cytometric analysis of C-kit phenotype in bone marrow. Control (FIG. 20A), IDPSC treatment 48 hours after irradiation (FIG. 20B), and irradiation without treatment with IDPSCs (FIG. 20C). FIG. 20D depicts the percentage of C-kit-positive cells. There is a slight decrease in C-kit-positive cells 48 hours after irradiation in relation to the control. There is an increase of C-kit-positive cells in the group treated with IDPSCs compare to the untreated group.

FIGS. 21A-21F depict immunostaining for C-kit in treated (FIGS. 21A, 21C, and 21E) and untreated (FIGS. 21B, 21D, and 21F) groups. Positive immunostaining for C-kit is observed in the cytoplasm of BM (FIGS. 21A-21B), the medullary aspirate cells cultured in vitro (FIGS. 21C-21D), and the extracellular matrix of the BM (FIGS. 21E-21F).

FIGS. 22A-22D depict representative histograms from flow cytometric analysis of CD34 phenotype in bone marrow analyzed in: control (FIG. 22A), IDPSC treatment 48 hours after irradiation (FIG. 22B), and irradiation without treatment with IDPSCs (FIG. 22C). FIG. 22D depicts the percentage of Sca-1-positive cells. There was a decrease of CD34-positive cells 48 hours after irradiation in relation to the control. There were increases in both experimental groups, with a greater increase in the IDPSC treated group.

FIGS. 23A-23F depict immunostaining of CD34 in treated (FIGS. 23A, 23C, and 23E) and untreated (FIGS. 23B, 23D, and FIG. 23F) groups. Positive immunostaining of CD34 is observed in the cytoplasm of BM (FIGS. 23A-23B), the medullary aspirate cells cultured in vitro (FIGS. 23C-23D), and the extracellular matrix of the BM (FIGS. 23E-23F).

FIGS. 24A-24D depict representative histograms from flow cytometric analysis of Sca-1 phenotype in bone marrow analyzed in: control (FIG. 24A), IDPSC treatment 48 hours after irradiation (FIG. 24B), and irradiation without treatment with IDPSCs (FIG. 24C). FIG. 24D depicts the percentage of C-kit-positive cells. There is a slight increase in Sca-1-positive cells 48 hours after irradiation in relation to the control. There is an increase of Sca-1-positive cells in the group treated with IDPSCs compared to the untreated group.

FIGS. 25A-25F depict immunostaining of Sca-1 in treated (FIGS. 25A, 25C, and 25E) and untreated (FIGS. 25B, 25D, and 25F) groups. Positive immunostaining of Sca-1 is observed in the cytoplasm of BM (FIGS. 25A-25B), the medullary aspirate cells cultured in vitro (FIGS. 25C-25D), and the extracellular matrix of the BM (FIGS. 25E-25F).

FIGS. 26A-26D depict representative histograms from flow cytometric analysis of CD45 phenotype in bone marrow analyzed in: control (FIG. 26A), IDPSC treatment 48 hours after irradiation (FIG. 26B), and irradiation without treatment with IDPSCs (FIG. 26C). FIG. 26D depicts the percentage of CD45-positive cells. There is a dramatic decrease in CD45-positive cells 48 hours after irradiation in relation to the control. There is a significant increase of CD45-positive cells in the group treated with IDPSCs compared to the untreated group.

FIGS. 27A-27F depict immunostaining of CD45 in treated (FIGS. 27A, 27C, and 27E) and untreated (FIGS. 27B, 27D, and 27F) groups. Positive immunostaining of CD45 is observed in the cytoplasm of BM (FIGS. 27A-27B), the medullary aspirate cells cultured in vitro (FIGS. 27C-27D), and the extracellular matrix of the BM (FIGS. 27E-27F).

FIGS. 28A-28D depict representative histograms from flow cytometric analysis of CD44 phenotype in bone marrow analyzed in: control (FIG. 28A), IDPSC treatment 48 hours after irradiation (FIG. 28B), and irradiation without treatment with IDPSCs (FIG. 28C). FIG. 28D depicts the percentage of CD44-positive cells. There is a dramatic decrease in CD44-positive cells 48 hours after irradiation in relation to the control. There is a significant increase of CD44-positive cells in the group treated with IDPSCs compared to the untreated group. Asterisks indicate statistical significance (ANOVA) between 48 hours after irradiation and control, P<0.05 (*). # indicates statistical significance in increasing expression of CD44-positive cells, P<0.05 (#).

FIGS. 29A-29F depict immunostaining of CD44 in treated (FIGS. 29A, 29C, and 29E) and untreated ((FIGS. 29B, 29D, and 29F) groups. Positive immunostaining for CD44 is observed in the cytoplasm of BM (FIGS. 29A-29B), the medullary aspirate cells cultured in vitro (FIGS. 29C-29D), and the extracellular matrix of the BM (FIGS. 29E-29F).

FIGS. 30A-30D depict representative histograms from flow cytometric analysis of CD90 phenotype in bone marrow analyzed in: control (FIG. 30A), IDPSC treatment 48 hours after irradiation (FIG. 30B), and irradiation without treatment with IDPSCs (FIG. 30C). FIG. 30D depicts the percentage of CD90-positive cells. There is a decrease in CD90-positive cells 48 hours after irradiation in relation to the control. There is an increase of CD90-positive cells in the group treated with IDPSCs compared to the untreated group.

FIGS. 31A-31F depict immunostaining of CD90 in treated (FIGS. 31A, 31C, and 31E) and untreated (FIGS. 31B, 31D, and 31F) groups. Positive immunostaining for CD90 was observed in the cytoplasm of BM (FIGS. 31A-31B), the medullary aspirate cells cultured in vitro (FIGS. 31C-31D), and the extracellular matrix of the BM (FIGS. 31E-31F).

FIGS. 32A-32F depict immunohistochemistry demonstrating positive labeling for anti-human antibody in BM of IDPSC treated groups (FIGS. 32A-32C) and an absence of any labeling in untreated groups (FIGS. 32D-32F). Arrows indicate immunopositive cells in the presence of the human anti-nucleus antibody.

FIGS. 33A-33C depict immunohistochemistry demonstrating positive labeling for anti-human antibody in mouse spleen of the IDPSC treated group. Arrows indicate immunopositive cells in the presence of the human anti-nucleus antibody.

FIGS. 34A-34C depict values of Erythrocytes (FIG. 34A), Hematocrit (FIG. 34B) and Leukocytes (FIG. 34C) in the groups treated with IDPSCs (D62 or 6 months) or untreated with IDPSCs (D62 or 6 months). The graphs show blood normalization tendency in all hematimetric indexes evaluated, with slower recovery in untreated animals. Graphs are generated by GraphPad Prism 5 software (GraphPad; PRISM, 2007).

FIGS. 35A-35D depict photomicrography of long-term BM group (6 months) stained with hematoxylin and eosin at a magnification of 100× and 200×. (FIGS. 35A-35B) the medullary canal is filled with hematopoietic elements in the treated group (6 months). (FIGS. 35C-35D) medullary hypocellularity with fat replacement (red arrow) in the long-term untreated group (6 months).

FIGS. 36A-36B depict percentage of p53-positive cells (FIG. 36A), Fibronectin-positive (FIG. 36B) in groups treated with IDPSCs (D62 or 6 months) or untreated with IDPSCs (D62 or 6 months).

FIGS. 37A-37B depict percentage of Nestin-positive cells (FIG. 37A), C-kit-positive (FIG. 37B) in groups treated with IDPSCs (D62 or 6 months) or untreated with IDPSCs (D62 or 6 months).

FIGS. 38A-38B depict percentage of CD105-positive cells (FIG. 38A), CD90-positive cells (FIG. 38B) in groups treated with IDPSCs (D62 or 6 months) or untreated with IDPSCs (D62 or 6 months).

FIGS. 39A-39B depict percentage of Sca-1-positive cells (FIG. 39A), CD34-positive (FIG. 39B) in groups treated with IDPSCs (D62 or 6 months) or untreated with IDPSCs (D62 or 6 months).

FIGS. 40A-40B depict percentage of CD45-positive cells (FIG. 40A), CD44-positive cells (FIG. 40B) in groups treated with IDPSCs (D62 or 6 months) or untreated with IDPSCs (D62 or 6 months).

FIGS. 41A-41D depict microphotography demonstrating positive effects of IDPSCs after ionic radiation in the mice treated with IDPSCs (FIGS. 41A-41B) or untreated with IDPSCs (FIGS. 41C-41D). (FIGS. 41A-41B) Notice the presence of hair (red arrow) and whiskers (white arrow). (FIGS. 41C-41D) Notice the absence of hair (red arrow) and whiskers (white arrow).

FIG. 42 depicts TNF-alpha secretion data. After irradiation (D2), the concentration of TNF-α increased compared to the control group (D0). After the first therapy (D17) there was difference between the hIDPSC treated and untreated groups, where the treated group had lower levels. In D32, although there was a significant difference between the groups, the levels were higher than in D17. One month after the third therapy (D62) the levels were lower than the control, without difference between them, but the group treated with less expressive values than non-treated group.

FIG. 43A-43B depict IL4 secretion data. (FIG. 43A) In D17, after the first therapy, IL-4 levels were higher in the hIDPSCs treated group, the same happens in D32. In D62, IL-4 levels stand out in the group that received the treatment with hIDPSC. (FIG. 43B). After the first therapy (D17), the hIDPSC treatment group showed higher levels in relation to the animals not treated. Subsequent to the second therapy (D32), IL-10 values remained similar to D17, in which the hIDPSC treatment group presented higher values than the untreated group. Already at the end of the test (D62), the treated group showed a significant increase in relation to the non-treated.

FIG. 44 depicts IL-17A secretion data. After irradiation (D2) the level of IL-17A was reduced in comparison to the control. After (D17), the level of IL-17A was statistically lower in compared to the untreated group. After the second therapy (D32) the hIDPSC treated group was maintained with lower IL-17A levels compared to the untreated group. At the end of (D62), once again the level of IL-17A was statistically lower in the hIDPSC treated compared to the untreated group.

### DETAILED DESCRIPTION OF INVENTION

The following description includes information that may be useful in understanding aspects of the disclosures. It is not an admission that any of the information provided herein is prior art relevant to the presently claimed inventions, or that any publication specifically or implicitly referenced is prior art.

As used herein, the verb “comprise” as is used in this description and the claims and its conjugations are used in its non-limiting sense to mean that items following the word are included, but items not specifically mentioned are not excluded. Also, reference to an element by the indefinite article “a” or “an” does not exclude the possibility that more than one of the elements are present unless the context clearly requires that there is one and only one of the elements. The indefinite article “a” or “an” thus usually means “at least one.”

As used herein, the term “patient” or “subject” is a mammal including but not limited to a human, mouse, rat, guinea pig, dog, cat, horse, cow, pig, or non-human primate, such as a monkey, chimpanzee, baboon or rhesus.

As used herein, the term “Matrigel” refers to a gelatinous protein mixture secreted by Engelbreth-Holm-Swarm (EHS) mouse sarcoma cells that resembles the complex extracellular environment found in many tissues.

A “harvesting cycle” constitutes a transfer of the DP to a new cell culture container after adherence and outgrowth of the IDPSCs followed by preservation (e.g. cryopreservation) and/or sub-culturing of the outgrowth of IDPSCs.

As used herein, “hypoxic conditions” comprise culturing the cells under culture conditions comprising or equivalent to: (i) a maximum of between about 0.5% and 1%, or a maximum of between about 0.5% and 15% oxygen (O₂); (ii) about 0.05%, 0.1%, 0.2%, 0.3%, 0.4%, 0.5%, 0.6%, 0.7%, 0.8%, 0.9%, 1% or 2% or 5% of oxygen (O₂); (iii) between about 0.1% and 2% oxygen (O₂); or any of (i), (ii) or (iii) with 5-7% CO₂.

As used herein, the term “stem cells” refers to immature, unspecialized cells that, under certain conditions, may differentiate into mature, functional cells.

As used herein, the term “IDPSC” or “hIDPSC” refers to an undifferentiated stem cell that is capable to differentiate into a wide spectrum of cell types, including but not limited to cells from central and periphery neural systems, e.g., neurons, astrocytes, ganglial cells, and/or oligodendrocytes.

As used herein, the terms “cell culture” or “cultured cell” refers to cells or tissues that are maintained, cultured, cultivated or grown in an artificial, in vitro environment.

As used herein, the term “pluripotent” refers to precursor cells that can form any adult cell.

As used herein, the term “treating” refers to medically caring for or dealing with a condition (such as a disease or an injury).

As used herein, the term “preventing” refers to medically caring for or dealing with a subject to inhibit or reduce the likelihood a condition (such as a disease or an injury) will occur.

As used herein, the term “undifferentiated” refers to cultured cells that display morphological characteristics of undifferentiated cells, distinguishing them from differentiated cells.

As used herein, the term “substantially homogenous” population of cells refers to a population of cells wherein the majority (e.g., between about 50% to about 90%) of the total number of cells have a specified characteristic of interest (for example, expression of CD44, CD90, Nestin, Vimentin, and Fibronectin).

As used herein, the term “co-express” refers to the simultaneous detection of two or more molecular markers, e.g., Nestin, Vimentin, and Fibronectin in the same cell population, preferably in/on the same cell.

As used herein, the terms “(cell) culture maintenance,” “proliferation,” “propagation,” “expansion,” and “growth,” refers to continued survival of a cell or population of cells with an increase in numbers of cells.

As used herein, the terms “bone marrow cellularity” refers to the volume ratio of hematopoiesis and fat.

As used herein with regard to cell cultivation and expansion, the term “large scale” refers to the isolation from DP and cultivation of SC under conditions, which permit at least the doubling of cells after each nonenzymatic harvesting cycle.

As used herein, the term “mesenchymal stem cells (MSCs)” refers to multipotent stromal cells that can self-renew by dividing and can differentiate into a variety of cell types, including osteoblasts (bone cells), chondrocytes (cartilage cells), myocytes (muscle cells) and adipocytes (fat cells that give rise to marrow adipose tissue).

As used herein, the term “condition” refers to an abnormal state of health that interferes with normal or regular feelings of wellbeing, which includes disease, i.e., a disorder of structure or function in the subject.

The present invention provides useful methods of treating, inhibiting, or preventing a condition (e.g., a disease or an injury) in a subject. The method comprises administering to a subject in need thereof a population of MSC-like cells isolated from a tissue of neural crest embryonic origin in an amount sufficient to treat, inhibit, or prevent the condition in the subject.

The present invention also provides a composition comprising a population of MSC-like cells isolated from a tissue of neural crest embryonic origin.

In some non-limiting embodiments, the condition leads to or derives from BMF. Non-limiting examples include aplastic anemia, fanconi anemia, dyskeratosis congenital, Shwachman-Diamond syndrome, Diamond-Blackfan anemia, severe congenital neutropenia (e.g., Kostmann syndrome), and congenital amegakaryocytic thrombocytopenia.

The BMF may be inherited or acquired. Non-limiting examples of the causes of acquired BMF include chemical injury, radiation exposure, chemotherapy, and immune system harms, etc. In certain non-limiting implementations, the dose of the radiation exposure is between 0.1-30 Gy, or any number range in between, e.g., 0.1-25 Gy, 0.1-20 Gy, 0.1-15 Gy, 0.1-10 Gy, 0.1-6 Gy, 0.5-25 Gy, 0.5-20 Gy, 0.5-15 Gy, 0.5-10 Gy, 0.5-6 Gy, 1-20 Gy, 1-15 Gy, 1-10 Gy, 1-6 Gy, 2-15 Gy, 2-10 Gy, 2-6 Gy, 4-10 Gy, and 4-6 Gy, etc.

Non-limiting examples of the subject include human, mouse, rat, guinea pig, or dog. In preferred embodiments, the subject is human, e.g., a victim of irradiation accident (e.g., exposed to atomic bomb or irradiation exposition accidents), a patient with myelogenous leukemia (e.g., 2-3 years following irradiation exposure), or a patient has BM inflammatory reaction.

In further non-limiting embodiments, the tissue of neural crest embryonic origin consists of a dental pulp from a tooth selected from a deciduous tooth, a permanent tooth, and a third molar.

The MSC-like cells are immature dental pulp stem cells (IDPSCs), e.g., human post-natal IDPSCs (hIDPSCs). The population of IDPSCs (e.g., hIDPSCs) may be an essentially phenotypically uniform population that is multifunctional. In some non-limiting implementations, the population of IDPSCs (e.g., hIDPSCs) is autologous. In other non-limiting implementations, the population of IDPSCs (e.g., hIDPSCs) is allogeneic. In preferred, non-limiting implementations, the IDPSCs (e.g., hIDPSCs) do not differentiate into intrinsic blood cells.

In preferred, non-limiting implementations, the IDPSCs (e.g., hIDPSCs) are capable of migrating to the bone marrow and/or the spleen of the subject. In other non-limiting implementations, the IDPSCs (e.g., hIDPSCs) migrate and home in bone marrow and/or spleen of the subject.

In other non-limiting implementations, a fraction of the population expresses at least one mesenchymal stem cell (MSC) markers. Non-limiting examples of the MSC markers include Nestin, CD105, CD90, CD73, CD44, CD29, CD117 (c-kit), Nestin, Vimentin, and Fibronectin, etc. In preferred, non-limiting implementations, the MSC marker comprises CD117 (c-kit) and/or Fibronectin. In other non-limiting implementations, the MSC marker comprises CD105, CD90, CD44, and Nestin.

The expression of above MSC markers is therapeutically important for BMF treatment. Nestin-positive stem cells exhibit angiogenesis potential and are essential for HSC maintenance. All cell types from the HSC niche, including endothelial cells, perivascular stromal cells, endosteal osteoblasts, and pericytes express Nestin, indicating that nestin-expressing cells are important for the HSC niche. CD90 is expressed in early-stage hematopoietic cells. CD90-positive cells mediate hematopoietic reconstitution and long-term engraftment and are involved in the improvement of human bone marrow transplantation efficiency. CD44 plays an important role in the maintenance of hematopoietic stem and progenitor pools, HSC migration, and homing. Fibronectin (FN) is a ubiquitous extracellular matrix (ECM) glycoprotein that plays a vital role in hematopoiesis: providing an anchorage and acting as a proliferative stimulus for erythroid and primitive progenitors. Vimentin is crucial for regulating monocyte/macrophage differentiation during blood cells maturation. Therefore, these five molecules may contribute to hematopoietic reconstruction in different manners.

In further non-limiting implementations, the fraction of the population lacks expression of CD34, CD45, CD14, and/or HLA ABC. In yet further non-limiting implementations, the fraction of the population expresses a low level of HLA ABC.

In some non-limiting implementations, the fraction is between 60% and 100%, or any percent number in between, e.g., 65-100%, 65-99%, 70-99%, 70-98%, 75-98%, 75-97%, 80-97%, 80-96%, 85-96%, 85-95%, 90-95%, 96-99%, and 90-100%. In other non-limiting implementations, the fraction is at least 60%, at least 65%, at least 70%, at least 75%, at least 80%, at least 85%, at least 90%, at least 95%, or at least 96%.

In a preferred, non-limiting implementation, 96-99% of the IDPSCs (e.g., hIDPSCs) are CD105⁺, CD73⁺, CD90⁺, CD29⁺, CD44⁺, CD117⁺ (c-kit⁺), Nestin⁺, Vimentin⁺, and Fibronectin⁺, CD45⁻, CD34⁻, HLA DW, and HLA ABC (low). Said biomarkers are defined cell signature fingerprints typical for MSCs with classic multipotent cell type characteristics.

In certain non-limiting embodiments, the composition is administered after a critical event selected from the group consisting of: radiation exposure, diagnosis of pancytopenia, diagnosis of bone marrow aplasia, diagnosis of bone marrow hypoplasia, and combinations thereof. The time interval between the critical event and the initial administration may be about 12 (e.g., 10-14) hours, about 18 (e.g., 14-22) hours, about 24 (e.g., 19-29) hours, about 30 (e.g., 24-36) hours, about 36 (e.g., 29-43) hours, about 48 (e.g., 38-58) hours, about 54 (e.g., 43-65) hours, about 60 (e.g., 48-72) hours, about 66 (e.g., 53-79) hours, about 72 (e.g., 58-86) hours, about 78 (e.g., 63-93) hours, about 84 (e.g., 67-101) hours, about 90 (e.g., 72-118) hours, or about 96 (e.g., 77-123) hours. In preferred, non-limiting implementations, the composition is administered 12 to 96 hours after the critical event (e.g., radiation exposure), or any number in between, e.g., 12-84 hours, 18-84 hours, 18-72 hours, 24-72 hours, 24-60 hours, 36-60 hours, or 36-48 hours, etc. In case of AA, the composition is administered about 12, about 18, about 24, about 30, about 36, about 48, about 54, about 60, about 66, about 72, about 78, about 84, about 90, or about 96 hours after human subject shows pancytopenia and/or bone morrow aplasia/hypoplasia.

In preferred, non-limiting embodiments, the administered IDPSCs (e.g., hIDPSCs) is in an amount sufficient to increase erythrocyte (RBC) counts, hematocrit levels, and/or leukocyte (WBC) counts in the bone marrow of the subject. For example, after irradiation, administered IDPSCs (e.g., hIDPSCs) is in an amount sufficient to normalize erythrocyte counts, hematocrit levels, and/or leukocyte counts. In some non-limiting implementations, the increase compared to non-treatment control is at least 5%, at least 10%, at least 15%, at least 20%, at least 25%, at least 30%, at least 40%, or at least 50%.

In some non-limiting implementations, the administered IDPSCs (e.g., hIDPSCs) is in an amount sufficient to improve platelet function, increase bone marrow fibroblast colony-forming units (CFU-F) after bone marrow ablation, promote recovery of bone marrow cellular components, and/or accelerate recovery of bone marrow cellular components. The composition may promote and/or accelerate recovery of bone marrow cellular components by increasing hematopoietic precursors and/or nucleated cells (e.g., megakaryocytes and stroma with different degrees of maturation).

In other non-limiting implementations, the administered IDPSCs (e.g., hIDPSCs) is in an amount sufficient to reduce bone marrow cell apoptosis in the subject, e.g., by reducing the number of p53-expressing cells. After irradiation, apoptosis occurs relating to the oncogene and anti-oncogene regulation. P53 is an important biomarker for apoptosis, and by decreasing the number of p53⁺ cells in the bone marrow may promote recovery of bone marrow milieu by an anti-apoptotic mechanism.

In further non-limiting implementations, the administered IDPSCs (e.g., hIDPSCs) is in an amount sufficient to modulate inflammatory reaction of organism in response to damages caused by irradiation and/or the BM injury or disease.

In yet further non-limiting implementations, the administered IDPSCs (e.g., hIDPSCs) is in an amount sufficient to increase Nestin-positive cells, CD44-positive cells, CD45-positive cells, CD105-positive cells, Sca-1-positive cells, and/or Nestin-positive cells, and/or reduce p53-positive cells in bone marrow of the subject. Sca-1-positive cells are HSC progenitors; CD105-positive cells are hematopoietic stem/progenitor cells; CD45-positive cells are hematopoietic lineage-restricted antigen cells; Nestin-positive cells support hematopoiesis (stromal) in the bone marrow HSC niche; and CD44-positive cells regulate hematopoietic progenitor distribution.

In some non-limiting implementations, the administered IDPSCs (e.g., hIDPSCs) is in an amount sufficient to facilitate the recovery of HSC/progenitors production and distribution within bone marrow of the subject and/or re-establish hematopoiesis in the subject's bone marrow. In other non-limiting implementations, the administered IDPSCs (e.g., hIDPSCs) is in an amount sufficient to promote BM vascular niche recovery and/or hematopoiesis reconstruction. AA impair medullary microenvironment and angiogenesis, decrease endothelial vessels by decreasing Nestin- and CD105-positive proliferative endothelial progenitor cells.

In further non-limiting implementations, the administered IDPSCs (e.g., hIDPSCs) is in an amount sufficient to increase regeneration of HSC niche components by increasing the number and/or the bone-marrow colony-forming-unit activity of intrinsic Nestin-positive MSCs. In further non-limiting implementations, the administered IDPSCs (e.g., hIDPSCs) is in an amount sufficient to promote regeneration of the stromal niche in the subject after receiving irradiation by enriching intrinsic CD105-positive cells, CD44-positive cells, and/or CD90-positive cells in the subject's bone marrow. Improving the enrichment of bone marrow with intrinsic endothelial Nestin- and CD105-positive progenitors promotes recovery of bone marrow vascular niche and angiogenesis in a subject after irradiation.

In some non-limiting implementations, the administered IDPSCs (e.g., hIDPSCs) is in an amount sufficient to promote hematopoietic niche recovery, e.g., by increasing proliferation of intrinsic BM cells, and/or increasing Sca-1-positive cells and/or preserving intrinsic, quiescent Sca-1-positive cells. Sca-1 plays a critical role in the activation of HSC and self-renewal of HSCs.

In other non-limiting implementations, the administered IDPSCs (e.g., hIDPSCs) is in an amount sufficient to promote HSCs niche recovery and hematopoiesis reconstruction, e.g., by increasing CD45-positive cells in the bone marrow of the subject. The hematopoietic stem cell (HSC) niche is a complex microenvironment that provides for the survival, expansion, and differentiation of quiescent HSCs as well as the migration of hematopoietic precursors to replenish the blood. HSC niche is composed of primitive HSCs, reticular cells, endothelial cells, bone-forming osteoblasts, and osteoclasts. Also, evidence revealed the role of the hematopoietic transmembrane tyrosine phosphatase, CD45, in HSC niche. CD45 is expressed in all leukocytes.

In yet other non-limiting implementations, the administered IDPSCs (e.g., hIDPSCs) is in an amount sufficient to increase homing of progenitor cells to the BM microenvironment and/or trafficking of cells within the BM compartment, e.g., by increasing CD44-positive cells. Myeloid and erythroid progenitor cells mediate lymphocyte activation and homing. These cells express an adhesion molecule, CD44. Adhesive interactions cause homing of progenitor cells to the BM microenvironment and likely contribute to trafficking of cells within the BM compartment. CD44 serves a variety of functions in human hematopoiesis.

In further non-limiting implementations, the administered IDPSCs (e.g., hIDPSCs) is in an amount sufficient to treat AA by promoting differentiation, recovering BM vascular niche, reducing apoptosis, modulating inflammatory reaction, and/or recovering bone marrow cellular components.

In yet other non-limiting implementations, the administered IDPSCs (e.g., hIDPSCs) is in an amount sufficient to promote the synthesis of Fibronectin and remodeling of extracellular matrix, to preserve the hearing and/or the ability to smell in the subject after radiation; to reduce side effects of irradiation, and/or to protect against the side effect of long-term irradiation or BMF by promoting weight increase. Radiation triggers hearing loss and temporary changes in smell. Cancer therapy uses irradiation intensively, especially in hematological malignancies. Irradiation inflicts damages to tissues surrounding the tumor, which may cause severe complications to patients. Human subjects frequently suffer weight loss from the diseases or injuries.

In further non-limiting implementations, the administered IDPSCs (e.g., hIDPSCs) is in an amount sufficient to lead to partial or complete BM hematopoiesis recovery in the subject, e.g., reverse a hematopoietic parameter counting after irradiation.

In further non-limiting implementations, the administered IDPSCs (e.g., hIDPSCs) is in an amount sufficient to promote remodeling of the extracellular matrix, e.g., by increasing fibronectin-positive fibroblast like cells and fibronectin with a characteristic extracellular matrix formation in an extensive network in the bone morrow of the subject. Extracellular matrix (ECM) is a dynamic and versatile compartment that supports organ development, function, and repairing. ECM is a relevant component of stem cell niche, which have a primary role in regulating stem cell properties, survival, motility, differentiation, and communication. Fibronectin, involved in cell adhesion, growth, migration, and differentiation, supports the normal function of an organism.

In yet further non-limiting implementations, the administered IDPSCs (e.g., hIDPSCs) is in an amount sufficient to enhance bone marrow regeneration, e.g., by reducing or eliminating fatty elements in bone marrow of the subject. Adipocytes have shown to be negative regulators of hematopoietic progenitor's cells thus, increased level of adipocytes in the marrow decrease the hematopoietic progenitor cells content leading to hematopoietic progenitor cell depletion.

In some non-limiting implementations, the administered IDPSCs (e.g., hIDPSCs) is in an amount sufficient to suppress intrinsic adipocytes development and/or enhance regeneration or hematopoietic recovery of bone marrow in the subject. In other non-limiting implementations, the administered IDPSCs (e.g., hIDPSCs) is in an amount sufficient to facilitate recovery of immunological health, thymus and spleen functions, and/or bone marrow hematopoiesis, e.g., by suppressing intrinsic adipocytes in the subject following irradiation. Since BMF is defined as the inability to produce the proper number of blood cells necessary to control immune function, the hematopoietic progenitor cells depletion would compromise immunologic health and affect the bone marrow, thymus, and spleen. The function of the thymus is to produce mature T cells, while spleen is an immunologic filter of the blood composed by B cells, T cells, macrophages, dendritic cells, natural killer cells and red blood cells.

In other non-limiting implementations, the administered IDPSCs (e.g., hIDPSCs) is in an amount sufficient to attenuate inflammation of the subject, e.g., by anti-inflammatory action by stabilizing IL-17A levels and/or increasing IL-4 and/or IL-10 secretion. Cytokine secretion modulates inflammation and decreased prokaryotic cytokine secretion inflammatory; stabilizing IL-17A levels prevents the progression of inflammation; and induction of IL-17A late response reduces chronic inflammation. In a preferred, non-limiting implementation, the IDPSCs are in an amount sufficient to reduce serum levels of TNF-α, IL-17A, or both in the subject. In another preferred, non-limiting implementation, the IDPSCs are in an amount sufficient to increase serum levels of IL-4, IL-10, or both in the subject.

In yet other non-limiting implementations, the administered IDPSCs (e.g., hIDPSCs) is in an amount sufficient to protective against irradiation and/or accelerate the recovery of hematopoiesis and the hematopoietic microenvironment in the subject's bone marrow.

In some non-limiting implementations, the increase, decrease, acceleration is by between 5% and 300%, or any percent number in between, e.g., 5-250%, 5-200%, 5-150%, 5-100%, 5-50%, 5-25%, 10-300%, 10-250%, 10-200%, 10-150%, 10-100%, 10-50%, 10-25%, 15-300%, 15-250%, 15-200%, 15-150%, 15-100%, 15-50%, 15-25%, 20-300%, 20-250%, 20-200%, 20-150%, 20-100%, 20-50%, 20-40%, 25-300%, 25-250%, 25-200%, 25-150%, 25-100%, 25-50%, 30-300%, 30-250%, 30-200%, 30-150%, 30-100%, 30-50%, 50-300%, 50-250%, 50-200%, 50-150%, or 50-100%, etc. In other non-limiting implementations, the increase, decrease, acceleration is by at least 5%, at least 10%, at least 15%, at least 20%, at least 25%, at least 30%, at least 35%, at least 40%, at least 45%, at least 50%, at least 100%, at least 200%, at least 300%, etc.

In certain non-limiting embodiments, the composition comprises between 10⁵ and 10¹⁰ IDPSCs (e.g., hIDPSCs), or any number range in between, e.g., 10⁵-10⁹, 10⁵-10⁸, 10⁵-10⁷, 10⁵-10⁶, 5×10⁵-10¹⁰, 5×10⁵-10⁹, 5×10⁵-10⁸, 5×10⁵-10⁷, 5×10⁵-10⁶, 10⁶-10¹⁰, 10⁶-10⁹, 10⁶-10⁸, 10⁶-10⁷, 5×10⁶-10¹⁰, 5×10⁶-10⁹, 5×10⁶-10⁸, 5×10⁶-10⁷, 10⁷-10¹⁰, 10⁷-10⁹, 10⁷-10⁸, 5×10⁷-10¹⁰ 5×10-10⁹ 10⁸-10¹⁰, 10⁸-10⁹, 5×10⁸-10¹⁰ or 10⁹-10¹⁰ cells. In a preferred, non-limiting implementation, the composition comprises 5×10⁶-10⁹ IDPSCs (e.g., hIDPSCs).

In some non-limiting embodiments, the IDPSCs (e.g., hIDPSCs) is between 0.5×10⁵ and 1.5×10⁹ per kg body weight of the subject per transplant, or any number in between, e.g., 0.5×10⁵-1.5×10⁸, 0.5×10⁵-1.5×10⁷, 0.5×10⁵-1.5×10⁶, 1×10⁵-1.5×10⁹, 1×10⁵-1.5×10⁸, 1×10⁵-1.5×10⁷, 1×10⁵-1.5×10⁶, 3×10⁵-1.5×10⁹, 3×10⁵-1.5×10⁸, 3×10⁵-1.5×10⁷, 3×10⁵-1.5×10⁶, 0.5×10⁶-1.5×10⁹, 0.5×10⁶-1.5×10⁸, 0.5×10⁶-1.5×10⁷, 0.5×10⁶-1.5×10⁶, 1×10⁷-1.5×10⁹, 1×10⁷-1.5×10⁸, 1×10⁷-1.5×10⁷, 3×10⁷-1.5×10⁹, 3×10⁷-1.5×10⁸, 0.5×10⁸-1.5×10⁹, 0.5×10⁸-1.5×10⁸, 1×10⁸-1.5×10⁹, 1×10⁸-1.5×10⁸, 0.5×10⁵-1.5×10⁵, 2×10⁵-3×10⁵, 4×10⁵-6×10⁵ 7×10⁵-8×10⁵ 0.5×10⁶-1.5×10⁶ 2×10⁶-3×10⁶ 4×10⁶-6×10⁶ 7×10⁶-8×10⁶, 0.5×10⁷-1.5×10⁷, 2×10⁷-3×10⁷, 4×10⁷-6×10⁷, 7×10⁷-8×10⁷, 0.5×10⁸-1.5×10⁸, 2×10⁸-3×10⁸, 4×10⁸-6×10⁸, 7×10⁸-8×10⁸, or 0.5×10⁹-1.5×10⁹ per kg body weight of the subject per transplant.

In other non-limiting embodiments, the IDPSCs (e.g., hIDPSCs) is about 1×10⁵ (e.g., 0.8×10⁵-1.2×10⁵), about 2.5×10⁵ (e.g., 2×10⁵-3×10⁵), about 5×10⁵ (e.g., 4×10⁵-6×10⁵), about 7.5×10⁵ (e.g., 6×10⁵-9×10⁵), about 1×10⁶ (e.g., 0.8×10⁶-1.2×10⁶), about 2×10⁶ (e.g., 1.6×10⁶-2.4×10⁶), about 2.5×10⁶ (e.g., 2×10⁶-4×10⁶), about 5×10⁶ (e.g., 4×10⁶-6×10⁶), about 7.5×10⁶ (e.g., 6×10⁶-9×10⁶), about 1×10⁷ (e.g., 0.8×10⁷-1.2×10⁷), about 2.5×10⁷ (e.g., 2×10⁷-3×10⁷), about 5×10⁷ (e.g., 4×10⁶-6×10⁶), about 7.5×10⁷ (e.g., 6×10⁷-9×10⁷), about 1×10⁸ (e.g., 0.8×10⁸-1.2×10⁸), about 2.5×10⁸ (e.g., 2×10⁸-3×10⁸), about 5×10⁸ (e.g., 4×10⁸-6×10⁸), about 7.5×10⁸ (e.g., 6×10⁸-9×10⁸), or 1×10⁹ (e.g., 0.8×10⁹-1.2×10⁹) per kg of the subject per transplant.

In some non-limiting embodiments, the IDPSCs (e.g., hIDPSCs) are administered in a pharmaceutically effective amount. As used herein, the phrase “pharmaceutically effective amount” refers to an amount sufficient to treat a disease, condition, or disorder. The level of the effective dosage can be determined according to the severity of the disease; the age, weight, health, and sex of a patient; the drug sensitivity in a patient; the administration time, route, and release rate; the treatment duration; or elements including drugs that are blended or simultaneously used with the composition of some embodiments, or other elements well-known in the medical field. For example, the dosage of the IDPSCs varies within a wide range and is determined according to the requirements of the individuals in specific cases. Generally, in the case of parenteral administration, the IDPSCs are usually administered in an amount of between 1×10⁵ and 1×10⁷, for example, 2×10⁵-8×10⁶, 4×10⁵-8×10⁶, 4×10⁵-6×10⁶ 6×10⁵-6×10⁶ 6×10⁵-4×10⁶ 8×10⁵-4×10⁶ 8×10⁵-3×10⁶ 9×10⁵-3×10⁶ 9×10⁵-2×10⁶, or 1×10⁶-2×10⁶. about 1×10⁷, about 1×10⁸, about 1×10⁹, or about 1×10¹⁰ cells/kg of a body weight of the individual. Further, the composition of some embodiments and other disease-treating substances known in the art can be simultaneously or sequentially administered to an individual.

In certain non-limiting embodiments, the composition is administered at least twice, at least three times, at least four times, or at least five times. In some non-limiting implementations, the composition is administered about once per month, about once per 2 months, about once per 3 months, about once per 4 months, about once per 5 months, about once per 6 months, about once per 7 months, about once per 8 months, about once per 9 months, about once per 10 months, about once per 11 months, about once per year, or about once per two years.

In other non-limiting implementations, the interval between administration is between 4 and 60 days, or any number in between, e.g., 4-50 days, 5-50 days, 5-40 days, 6-40 days, 6-30 days, 8-30 days, 8-20 days, or 10-20 days, etc. In yet other non-limiting implementations, the interval between administration is about 5 (e.g., 4-6) days, about 10 (e.g., 8-12) days, about 15 (e.g., 12-18) days, about 20 (e.g., 16-24) days, about 25 (e.g., 20-30) days, about 30 (e.g., 24-36) days, about 45 (e.g., 36-54) days, about 60 (e.g., 48-72) days. In further non-limiting implementations, three administrations equal one cycle, repeated after about 1 month, about 2 months, about 3 months, about 4 months, about 5 months, about 6 months, about 7 months, about 8 months, about 9 months, about 10 months, about 11 months, about one year, or about two years.

In certain non-limiting implementations, one or more clinical improvements are observed after the 1st, the 2nd, or the 3rd administration of the composition. In other non-limiting implementations, the one or more clinical improvements last at least 1 month, at least 2 months, at least 3 months, at least 4 months, at least 5 months, or at least 6 months. In further non-limiting implementations, no apparent reversion of the recovery of hematopoiesis is observable.

In some non-limiting implementations, the composition comprising allogeneic IDPSCs is administered at least three times with an about one-month interval do not induce immunological responses without applying immunosuppressive protocols.

In some non-limiting embodiments, the administered IDPSCs (e.g., hIDPSCs) is in an amount sufficient to provide long-term (e.g., six-month) and stable hematopoietic recovery, by improving the function of stromal, hematopoietic, and vascular niche, extracellular matrix remodeling, suppression of adipose tissue development, anti-apoptotic action within bone marrow, continuously providing protection of hematopoiesis, angiogenesis, and/or stimulation of cell proliferation.

In further non-limiting embodiments, the composition further comprises bone-marrow stem-cells. In some non-limiting implementations, the bone-marrow stem-cells are autologous. In other non-limiting implementations, the bone-marrow stem-cells are allogeneic. In yet further non-limiting embodiments, the composition further comprises hematopoietic stem cells (HSCs). In some non-limiting implementations, the HSCs are autologous. In other non-limiting implementations, the HSCs are allogeneic. In a preferred non-limiting embodiment, the HSCs is between 1×10⁶/kg and 1×10/kg of the subject per transplant. In further non-limiting implementations, the IDPSCs, HSCs, and/or BM mononuclear cells are administered in an amount sufficient to protect the bone marrow from degenerative clinical damage, loss of tissue or cellular functioning. In yet further non-limiting implementations, MSCs with autologous or allogeneic HSCs leads to more robust engraftment and better therapeutic effects than co-transplanting MSCs with autologous or allogeneic mononuclear BM cells.

The IDPSCs (e.g., hIDPSCs) may be administrated intravenously, intraperitoneally, or using intraosseous infusion that is the process of injecting directly into the bone. Irradiation activates molecular pathways that increase the release of tissue chemokines, which attract MSCs to injured tissues. In certain embodiments, IDPSCs are intravenously or intraperitoneally administered which allows IDPSCs to migrate to BM and prevent the inflammatory reaction. Intraosseous infusion provides a non-collapsible entry point into the systemic venous system. This technique can also be used to provide fluids and medication when intravenous access is not available or not feasible. The FDA approved automatic intra-osseous devices for fluid and drug administration, providing quick and safe access to the patient's vascular system. In some non-limiting implementations, the IDPSCs (e.g., hIDPSCs) is administrated intravenously. In other non-limiting implementations, the IDPSCs (e.g., hIDPSCs) is administrated using intraosseous infusion.

In some non-limiting embodiments, the compositions is administered locally or systemically, e.g., require ex vivo or in vivo manipulations to obtain therapeutic activity to induce a local effect (e.g., a direct regenerative effect of IDPSCs in contact with degenerative tissue affecting cell proliferation/cell death, tissue integrity, and functionality) and/or due to a systemic effect (e.g., by releasing protective trophic factors and reducing levels of pro-inflammatory cytokines).

Non-limiting examples of the administration routes include known medical methods of treating BMF, degenerative, reproductive clinical damage, and/or to preventing subject, tissue, cells from future damage. IDPSC treatment herein encompasses abrogating, substantially inhibiting, slowing or reversing the progression of a condition, substantially ameliorating clinical symptoms or substantially preventing or reducing the appearance of clinical symptoms.

According to some aspects, a subject having BMF is administered with undifferentiated IDPSCs. According to other aspects, a subject having BMF is administered with IDPSCs induced to undergo special differentiation (progenitors). IDPSCs, for example, may be injected directly into the disease loci/cavity/milieu, transplanted elsewhere in the body, or may be adhered to a scaffold or delivery vehicle and surgically inserted/transplanted/grafted into an acceptable location in the body. Typically, IDPSCs are administered at a therapeutically effective amount that is sufficient to prevent or treat BMF through various administration routes including intravenous, Intraperitoneal, and intraosseous infusion, which is preferential.

In some non-limiting embodiments, the population of IDPSCs (e.g., hIDPSCs) is prepared by: a) obtaining a dental pulp (DP); b) washing the DP with a solution containing antibiotics; c) establishing an explant culture by placing the DP onto a plastic surface in a culture medium; d) mechanically transferring the DP onto a different plastic surface in the culture medium; e) repeating steps (c) and (d); and f) collecting the hIDPSCs that adhere to the plastic surface from the explant culture. In further non-limiting implementations, the preparation of the population of hIDPSCs further comprising passaging the explant culture of hIDPSCs from step c) prior to collection, wherein passaging comprises enzymatically treating the IDPSCs (e.g., hIDPSCs) and expanding the explant culture. In yet further non-limiting implementations, the population of IDPSCs (e.g., hIDPSCs) comprises IDPSCs after 2, 3, 4, 5, 6, and/or 7 passages. In preferred implementations, the population of IDPSCs (e.g., hIDPSCs) comprises IDPSCs after 4 and/or 5 passages.

In some non-limiting implementations, the population of IDPSCs (e.g., hIDPSCs) is obtained after at least 3, 4, 5, 6, and/or 7 harvesting cycles. In preferred implementations, the population of IDPSCs (e.g., hIDPSCs) is obtained after at least 5 harvesting cycles.

In other non-limiting embodiments, the population of IDPSCs (e.g., hIDPSCs) is cultured under normoxic or hypoxic conditions. In some non-limiting implementations, the percent oxygen (O₂) is between about 0.05% and 15%, or any percent number in between, e.g., 0.05-10%, 0.05-5%, 0.05-2%, 0.05-1%, 0.1-15%, 0.1-10%, 0.1-5%, 0.1-2%, 0.1-1%, 0.2-15%, 0.2-10%, 0.2-5%, 0.2-2%, 0.2-1%, 0.4-15%, 0.4-10%, 0.4-5%, 0.4-2%, 0.4-1%, 0.5-15%, 0.5-10%, 0.5-5%, 0.5-2%, 0.5-1%, 0.6-15%, 0.6-10%, 0.6-5%, 0.6-2%, 0.6-1%, 0.7-15%, 0.7-10%, 0.7-5%, 0.7-2%, 0.7-1%, 0.8-15%, 0.8-10%, 0.8-5%, 0.8-2%, 0.8-1%, 0.9-15%, 0.9-10%, 0.9-5%, 0.9-2%, 0.9-1%, 1-15%, 1-10%, 1-5%, 1-2%, 2-15%, 2-10%, 2-5%, 5-15%, or 5-10%, etc. In other non-limiting implementations, the percent oxygen (O₂) is about 0.05% (e.g., 0.04-0.06%), about 0.1% (e.g., 0.08-0.12%), about 0.2% (e.g., 0.16-0.24%), about 0.3% (e.g., 0.24-0.36%), about 0.4% (e.g., 0.32-0.48%), about 0.5% (e.g., 0.4-0.6%), about 0.6% (e.g., 0.48-0.72%), about 0.7% (e.g., 0.56-0.84%), about 0.8% (e.g., 0.64-0.96%), about 0.9% (e.g., 0.72-1.08%), about 1% (e.g., 0.8-1.2%), about 2% (e.g., 1.6%-2.4%), or about 5% (e.g., 4%-6%) of oxygen (O₂), etc.

In some non-limiting embodiments, the population of IDPSCs (e.g., hIDPSCs) is prepared using Cellavita technology followed by an in vitro expansion. In other non-limiting embodiments, the population of IDPSCs (e.g., hIDPSCs) is minimally manipulated young stem cells at low passages and PDs without losing their stemness.

Various embodiments of the contemplated disclosures are further illustrated by the following examples that should not be construed as limiting. The contents of all references, patents, and published patent applications cited throughout this application, as well as the Figures, are incorporated herein by reference in their entirety for all purposes.

### EXAMPLES

Aspects of this disclosures described herein relate to a method of BMF treatment using unique and mixed population of multipotent human adult stem cells derived from dental germ (the dental germ are primitive embryonic cells that are the precursors of teeth), primary dental pulp of deciduous, third molar, and permanent teeth (see Lizier et al. (2012) PLoS ONE 7:e39885).

Said mixed population comprises MSCs, pericytes, and neuro-epithelial capable of differentiating into endoderm, ectoderm, and mesoderm embryonic germ layers.

A further embodiment relates to a method of continuous derivation of IDPSCs from the prolonged cultivation of dental DP explant ex vivo through multiple harvesting cycles.

Said method allows an unexpected and almost unlimited number of harvesting/derivation cycles and therefore may reduce passage number of IDPSCs to obtain a similar number of cells as previously reported methods.

Said disclosed method is easy to perform and diminishes the probability of occurrence of spontaneous genomic mutations and eventual karyotype abnormalities. Spontaneous genomic mutations or karyotype abnormalities may arise during multiple passages in stem cells, which occur in traditional methods for derivation of IDPSCs using enzymatic digestion of dental pulp.

The number of stem cells that present an MSC/pericyte phenotype is diverse in different populations of MSCs from dental tissue, and it presents individual variations. Therefore, a significant advantage for single donor-derived culturing for multiple batches includes reduced variability, consistency, and reproducibility.

As an example, using said method it is possible to derive a therapeutically sufficient quantity of low passage IDPSCs derived from a single donor's DPs which may be obtained from at least one deciduous tooth or optionally four upper teeth and four lower “milk” teeth that may be stored or preserved and used as needed.

An additional embodiment relates to a method of wherein a clinically sufficient number of IDPSCs can be obtained at low passage number.

Stem cells are self-renewing and can differentiate into multi-lineage cells. They are divided into three principal classes: embryonic stem cells (ESCs), induced pluripotent stem cells (iPSCs), and adult stem cells. Mesenchymal stem cells (MSCs) are adult stem cells, which can be found in bone marrow, umbilical cord (UC) blood and the Wharton's jelly, adipose tissue (AT), dental pulp (DP) tissue, amniotic fluid, and other fetal and postnatal tissues. MSCs are the non-hematopoietic, multipotent stem cells able to differentiate into mesodermal lineages such as osteocytes, adipocytes, and chondrocytes. MSCs express cell surface markers like a cluster of differentiation CD73, CD90, CD105, CD29, and CD44. MSCs lack the expression of CD34, CD45, CD14, and HLA (human leucocyte antigen)-DR. For the first time, these cells were obtained from bone marrow.

MSCs is an effective tool in the treatment of chronic diseases, due to immunomodulatory properties, capacity to secrete cytokines and immune-receptors that regulate the microenvironment in the host environment. MSCs have different functional purpose when compared with ESCs and iPSCs, which is limited by tissue regeneration. The major challenge of using MSCs in cell therapy and regenerative medicine is to obtain great cell number without losing their potency during sub-culturing and at higher passages. It has been shown, for example, that human BM and adipose tissue-derived MSCs become senescent, exhibiting restricted differentiation potential, shortening of the telomere length, and morphological alterations when they were progressively cultured at higher passages and high population doubling (PD) time. Furthermore, long-term culturing of MSCs increases the probability of mutation occurrence and malignant transformation.

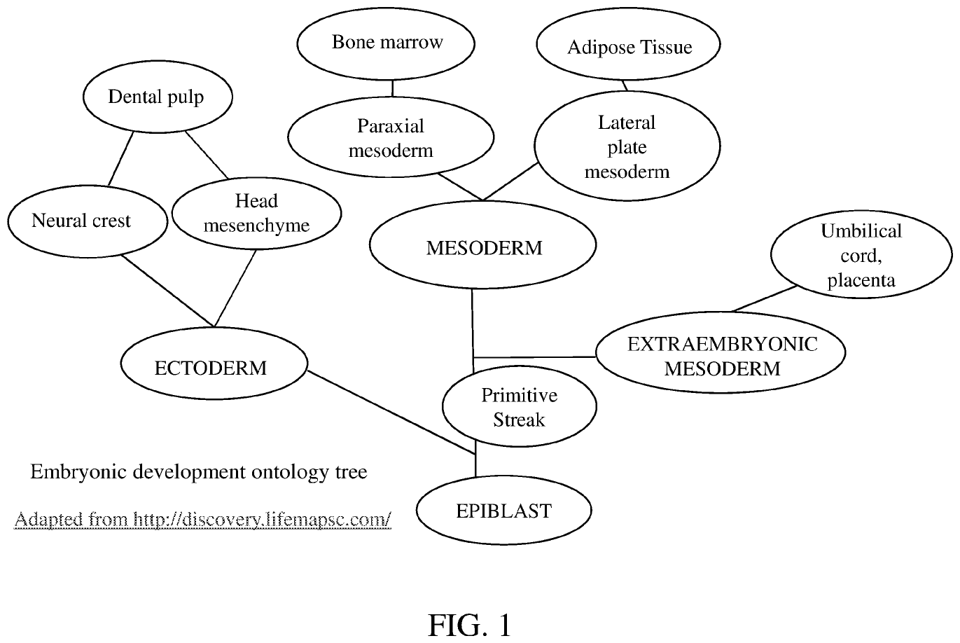

Moreover, although MSCs belong to the same class of stem cells—adult, they are derived from different sources and can possess a distinct differentiation potential and biological function, which depends on their embryonic and adult tissue origin (FIG. 1). Moreover, profound differences in development potential between MSCs sources were found, which might implicate with MSCs clinical use. Accordingly, it was recently postulated “MSCs from different sources have radically different differentiation properties” (Sacchetti et al., 2016). MSCs are isolated from tissues which may possess mesoderm, endoderm, ectoderm, and extraembryonic mesoderm embryonic origin. Bone marrow and adipose tissue-derived MSCs are originated from mesoderm, while MSC derived from, for example, umbilical cord and placenta are of extraembryonic mesoderm origin. Dental pulp stem cells are of ectoderm origin; more precisely, they are derived from neural crest (NC). In contrast to mesoderm, the NC is the source of a variety of MSCs termed mesectoderm or ectomesenchyme. Thus, MSCs isolated from different sources are desirable for providing products for BMF treatment.

Hierarchically mesectoderm is anterior to the ectomesenchyme. Mesectoderm originates of the formation of the meninges and becomes pigment cells. Ectomesenchyme is a key piece for the formation of the hard and soft tissues of the head and neck such as bones, muscles, teeth, and the pharyngeal arches. Prior to the present invention, neither mesectoderm nor ectomesenchyme was known to contribute to hematopoietic cells formation or to support or regulate hematopoiesis. Stromal cells and hematopoietic stem cells are derived from embryonic mesoderm and found in the red bone marrow, which is a component of the most bones core. Therefore, while mesoderm origin is known to be essential for BMF treatment, it is unexpected that stem cells of mesectoderm or ectomesenchyme were able to support and regulate hematopoiesis.

Advantages for using immature dental pulp stem cells (IDPSCs)-Cellavita™ (U.S. Pat. No. 9,790,468) for the treatment of BMF include the ability to greatly expand the IDPSCs in culture after isolation following Cellavita technology. This technology avoids sub-culturing at higher passages and high population doubling (PD) time as well as induction of senescing, restricted differentiation potential, shortening of the telomere length, morphological alterations, and probability of malignant transformation.

Unlike MSC from other adult sources that do not propagate well in culture, IDPSCs may be greatly expanded in culture and retain the ability to differentiate into a plurality of cell products. For example, one dental pulp may provide one lot of IDPSCs enough to treat a large number of patients (˜35-100), which also helps to standardize clinical outcomes.

Described herein are the distinct pattern and characteristics of a mixed population of human immature dental pulp stem cells isolated from their niche localized in the central region of the coronal and radicular pulp, which contains large nerve trunks and blood vessels. More specifically this region can be identified as the innermost pulp layer, which is a cell-rich zone and contains fibroblasts and undifferentiated stem cells in close association with blood vessels. A new immature dental pulp stem cell phenotype is described and characterized by the expression of hallmarks of mesenchymal-like, limbal-like and neuronal/neuroepithelial-like stem cells. The IDPSCs are multipotent cells, which can produce several type of terminally differentiated cells derivative of the three germ layers, the endoderm, ectoderm, and mesoderm.

During development and in an adult organism, stem cells can be found in a special microenvironment called a stem cell niche. A niche is a specialized anatomic site, which interacts with stem cells. It is hypothesized that a stem cell niche prevents differentiation and thus regulates their fate. During embryonic development, niche factors act on embryonic stem cells inducing their proliferation or differentiation. These changes occur due to alteration of genes and specific proteins expression leading to the development of the fetus and formation of the entire organism. During the post-natal period of human life, especially in childhood, stem cell niches continuously produce specialized cells that are necessary to complete development. In an adult organism, the niche maintains adult stem cells in a quiescent state. Stem cell activation occurs in response to tissue injury when the surrounding microenvironment is sending signals to stem cells to promote self-renewal, proliferation, and differentiation to restore damaged tissues. Several factors are involved in regulation of stem cell behavior within the niche. First, cell-cell interactions between stem cells as well as interactions between stem cells and neighboring differentiated cells, interactions between stem cells and adhesion molecules, extracellular matrix components, the oxygen tension, growth factors, cytokines, and the physiochemical nature of the environment including the pH, ionic strength (e.g., Ca²⁺ concentration), and metabolites like ATP are also important. Such reciprocal interaction between stem cells and the niche occurs during development and is maintained during adulthood.

Knowledge about stem cell niches in vivo is very relevant because it can help develop cell culture in vitro conditions and to preserve cell stemness after isolation. Identification of such niches within different tissues and discovery of their components and functioning is essential for regenerative therapies. This knowledge will provide information about various components, which is important for cell proliferation and differentiation during stem cells in vitro growing, expansion and differentiation, which must be controlled in flasks or plates to provide a sufficient quantity of the proper cell type prior to being introduced back into the patient for therapy. Adult stem cells remain in an undifferentiated state throughout adult life. However, when they are cultured in vitro, they often undergo an “aging” process in which their morphology is changed, and their proliferative capacity decrease. Correct culturing conditions of adult stem cells need to improve so that adult stem cells can maintain their stemness over time.

Two multipotent stem cell populations, HSC and MSC, compose the bone marrow. The HSCs control the immune cell lineages, and the MSCs give rise to bone, cartilage, muscle, tendon, ligament and fat cells. In an adult organism, HSCs of bone marrow have their niche composed of subendoosteal osteoblasts, sinusoidal endothelial cells, and bone marrow stromal cells, such as fibroblasts, monocytes, and adipocytes.

Recently, two types of MSCs in bone marrow of long bones with specialized HSC niche function were described. These MSCs comprise different populations and have distinct functions. Thus, mesoderm-derived Nestin negative cells lose their activity soon after birth. In contrast, Nestin-positive neural crest-derived quiescent cells preserve their MSC activity in adulthood differentiating into HSC niche-forming MSCs. These Nestin positive cells help to set up the HSC niche by secreting stromal cell-derived factor 1 (SDF1), also named as CXCL12, known to maintain the HSC population and lymphoid progenitors within the bone marrow. Both populations are dependent on one another for survival.

The studies showed that in the case of bone marrow transplantation, HSCs co-transplanted with MSCs demonstrate better engrafting. Moreover, recent data suggest that bone marrow MSCs have regulatory factors that control the HSC proliferative capacity.

Dental pulp stem cells are a kind of somatic stem cells which is present in dental pulp tissue inside the dentine of teeth, and which is capable of differentiating into dental pulp, dentine and the like (capable of differentiating mainly into odontoblasts). Dental pulp stem cells can be obtained by extirpating dental pulp tissue from (i) a tooth extracted for the sake of convenience in orthodontic treatment or a tooth extracted because of periodontal disease and the like, or from (ii) a wisdom tooth (aka a third molar) extracted for the sake of convenience in orthodontic treatment or of treatment of wisdom tooth periodontitis and the like.

Although any tooth retaining dental pulp tissue can be used as a source of dental pulp stem cells, it is preferable to select a tooth that is rich in dental pulp stem cells with a high potential for proliferation.

One suitable source of dental pulp stem cells is dental pulp tissue derived from a wisdom tooth of a young person (for example, in humans, about 12-16 years) having the wisdom tooth extracted for orthodontic purposes. Wisdom teeth at these ages are still in the midst of dental root formation during the initial stage of dental differentiation and are characterized by a high abundance of dental pulp tissue, a relatively high density of dental pulp stem cells, and a very high potential for their proliferation.

Because wisdom teeth are sometimes extracted for orthodontic purposes in other age groups, and also because dental pulp tissue can be obtained from teeth other than wisdom teeth, extracted for the sake of convenience, the availability of these extracted teeth is high.

Other potential sources of dental pulp stem cells include teeth extracted for the treatment of periodontal disease, wisdom teeth extracted because of wisdom tooth periodontitis and the like. In this case, there are disadvantages of an increased risk of contamination and a smaller amount of dental pulp tissue obtained. Because of the ease of obtaining these teeth from adults (particularly the elderly), however, these materials can serve as a major source of dental pulp stem cells when autologous transplantation of cells or tissue differentiated from these cells is desired.

The dental pulp stem cells that can be used in the present invention may be derived from mammals. Although dental pulp stem cells can be collected from any animal species, it is particularly preferable that the dental pulp stem cells be collected from the patient or from another person sharing the same type of human leukocyte antigen (HLA) because of the absence of graft rejection, when the stem cells obtained are used for human regenerative medicine. When the stem cells are not administered (i.e., transplanted) to a human, but are used as, for example, as a source of cells for screening to determine the presence or absence of the patient's drug susceptibility and adverse drug reactions, the dental pulp stem cells must be collected from the patient or from another person sharing the same gene polymorphism correlating to the drug susceptibility and adverse drug reactions.

Various companies have set up of tooth banking to isolate MSCs/pericytes from dental tissues and to tap the potential of this new and innovative approach for preserving stem cells from deciduous teeth and other dental sources. In the USA, StemSave, BioEden, and Store-A-Tooth are companies involved in banking of tooth stem cells. In Japan, the first tooth banks were established at Hiroshima University and Nagoya University.

### Example 1. Experimental Procedures

Immature Dental Pulp Stem Cells

Dental pulp was extracted from normal exfoliated human deciduous teeth of 5- to 7-year-old children (10 patients) under local with informed consent of the patients. IDPSC were obtained from human DP using explant method. For IDPSC isolation we used culture medium described previously in Kerkis et al., 2006. Briefly: Dulbecco's modified Eagle's medium (DMEM)/Ham's F12 (1:1, Invitrogen, Carlsbad, Calif., USA) supplemented with 15% fetal bovine serum (FBS, HyClone, Logan, Utah, USA), 100 U/ml penicillin, 100 μg/ml streptomycin, 2 mM L-glutamine, and 2 mM nonessential amino acids.

Further for IDPSC cultivation a basal culture medium described above was changed to Dulbecco's modified Eagle's medium (DMEM)/Ham's F12 (1:1, Invitrogen, Carlsbad, Calif., USA) supplemented with 10% fetal bovine serum (HyClone, Logan, Utah-USA), 1% penicillin and streptomycin (Gibco) and 1% non-essential amino acids (ANE-Gibco). DMEM/F12 has L-glutamine in its formulation. It was observed that supplementing the media with additional L-glutamine increases stem cells aggregation, which could clog the vein during stem cells transplantation, and might even cause the death of the recipient.

The cells were obtained after 5 dental pulp transfers, no pooling was made and used for inoculation at passages 4 or 5, preferentially. The cells were incubated at 37° C. in a 5% CO₂ and high humidity environment.

Hemogram

Blood collection was performed using the BD Vacutainer® tube (BD Diagnostics, Sao Paulo, SP, Brazil), next 250-500 μL with EDTA K2 was added to preserve cellular morphology. After the collection, the cell elements were counted using the automated hematology analyzer mindray BC-2800Vet®.

Morphological analyzes were carried out using blood smears stained with Panta Instant Pro NewProv® (NewProv—Produtos para Laboratorio, Pinhais, PR., Brazil) and also using the Nikon DS-Ri1 microscope. In addition, the clinical picture of the animals was evaluated throughout the experiment, adding and complementing the data obtained by the automatic counter.

Medullary Aspirate Collection

The medullary aspirate was collected from the femur and humerus bones after the euthanasia of the animals. To perform this procedure the bone was dissected, and the proximal epiphyses were removed, and the spinal contents were collected with the aid of a syringe (1 mL) and a needle (13×4.5-26G½ BD®). After collection of the spinal aspirate, absolute cell counts were performed in the Newbauer chamber, and aliquots of the BM samples were used for the CFU-F, flow cytometry, and BM smear assays, according to each protocol.

Test Colony Forming Unit (CFU-F)

Cells derived from BM aspiration were subjected to the CFU-F assay to compare the clonogenic capacity of BM cells treated with DPSC and the untreated group. Cells were counted in Neubauer's chamber and 4×10⁵ of viable cells were seeded in triplicate in cell culture dish (33 mm ø) TPP® (Techno Plastic Products, Zollstrasse, Trasadingen, Switzerland). DMEM-Low Glucose Gibco™ culture medium (Life Technologies, Carlsbad, Calif., USA) containing 10% Fetal Bovine Serum 1% non-essential amino acids, 1% L-Glutamine and 1% antibiotic (100 U/ml penicillin and 100 ng/ml streptomycin) Invitrogen™ (Invitrogen Corporation Carlsbad, Calif., USA), were used and the cells were maintained in a humidified atmosphere at 37° C. in 5% CO₂. Cells were maintained for 21 days in culture medium changed every three days. After 21 days, the cells were fixed in 4% paraformaldehyde solution for 24 hours. The cells were then dehydrated in a battery of 100%, 95%, 80%, and 50% alcohols for 3 minutes each, washed with distilled water and then stained with 1% Merck® crystal (Merck Millipore, Darmstadt, Germany) for 10 minutes to count the colonies formed.

Flow Cytometry

Aliquots of 1×10⁶ BM cells aspirated previously were fixed in 0.1% paraformaldehyde for 20 minutes, next the cells were washed twice with PBS for 5 minutes. Next, 0.1% Sigma® Triton X-100 (Sigma-Aldrich, Saint Louis, Mo., USA) was added for 20 minutes in the aliquots that to provide cells permeabilization. The cells were again centrifuged in PBS for 5 minutes at 2000 RPM. The cells were then incubated with 5% BSA solution (Sigma®) to block non-specific binding (for 30 minutes). Subsequently, the cells were washed 1×PBS for 5 minutes and incubated with the primary antibodies described in Table 1 overnight. The cells were then washed 1×PBS for 5 minutes for removal of the primary antibody, and incubated with the secondary antibodies described in Table 2 (Appendix B) for 1 hour. Finally, the samples were washed one more time for 5 minutes. The supernatant was discarded, and the pellet was resuspended in 100 μl PBS, and the cells were read and analyzed by the BD Accuri® c6 Flow Cytometer. The results were analyzed by the software FlowJo V.10.1.

TABLE 1
Primary antibodies used in flow cytometry (FACS) assays to characterize BM
cells aspiration, in immunohistochemistry (IHC) to characterize medullary
tissue, in immunocytofluorescence (ICF) to characterize BM cells in vitro,
and in immunocytochemistry (ICQ) to characterize the BM aspirate cells.

| Antibody | Species | Brand | Dilution | FACS | IHQ | ICF | ICQ |
|---|---|---|---|---|---|---|---|
| CD 34 | Goat | Santa Cruz | 1:100 | X | X | X | X |
| | | Biotechnology ® | | | | | |
| CD 44 | Rat | AbCam ® | 1:100 | X | X | X | X |
| CD 45 | Rat | AbCam ® | 1:100 | X | X | X | X |
| CD 90 | Rat | AbCam ® | 1:100 | X | X | X | X |
| CD 105 | Rabbit | AbCam ® | 1:200 | X | | | |
| C-kit | Rabbit | Santa Cruz | 1:100 | X | X | X | X |
| | | Biotechnology ® | | | | | |
| Fibronectin | Rabbit | Dako ® | 1:200 | X | X | X | X |
| Nestin | Rabbit | AbCam ® | 1:200 | X | X | X | X |
| Human Nucleus | Mouse | AbCam ® | 1:500 | | X | | |
| p53 | Rabbit | AbCam ® | 1:200 | X | | | |
| Sca-1 | Rat | Abcam ® | 1:100 | X | X | X | X |
| Vimentin | Goat | Santa Cruz | 1:100 | | | X | |
| | | Biotechnology ® | | | | | |

TABLE 2
Secondary antibodies used in flow cytometry (FACS) assays to characterize
BM cells, in immunohistochemistry (IHC) to characterize medullary tissue,
in immunocytofluorescence (ICF) to characterize BM cells in vitro, and
in immunocytochemistry (ICQ) to characterize the BM aspirate cells.

| Antibody | Species | Brand | Dilution | FACS | IHQ | ICF | ICQ |
|---|---|---|---|---|---|---|---|
| Anti-Goat IgG (H + L) (Alexa | Goat | ThermoFisher | 1:200 | X | | | |
| Fluor ® 647 conjugate) | | Scientific ® | | | | | |
| Anti-Goat IgG (FITC | Goat | Santa Cruz | 1:100 | | | X | |
| conjugate) | | Biotechnology ® | | | | | |
| Anti-Goat IgG (HRP | Goat | Dako ® | 1:100 | | X | | X |
| conjugate) | | | | | | | |
| Anti-Rabbit IgG (H + L) (Alexa | Rabbit | ThermoFisher | 1:200 | X | | | |
| Fluor ® 633 conjugate) | | Scientific ® | | | | | |
| Anti-Rabbit IgG (FITC | Rabbit | Santa Cruz | 1:100 | | | X | |
| conjugate) | | Biotechnology ® | | | | | |
| Anti-mouse/Anti-Rabbit | Mouse/ | Dako ® | — | | X | | X |
| Envision + Dual Link (HRP | Rabbit | | | | | | |
| conjugate) | | | | | | | |
| Anti-Rat IgG (FITC conjugate) | Rat | Santa Cruz | 1:100 | X | | X | |
| | | Biotechnology ® | | | | | |
| Anti-Rat IgG (HRP conjugate) | Rat | Dako ® | 1:100 | | X | | X |

Collection of Medullary and Spleen Tissue

After the euthanasia of the animals, collections of the medullary and spleen tissue were performed for the histological and immunohistochemical analyzes, for this purpose the samples were fixed in 10% paraformaldehyde (Sigma®).

Decalcification

After fixation, the bones were decalcified using the solution that contains 5.5 g EDTA (Sigma®), 10 ml Formol (Sigma®) and 90 ml distilled water. The material was covered with the solution at least 40 times and was changed every 3 days for 2 weeks.

Histochemistry

The histology slides were stained by the hematoxylin-eosin method (Merck®) to evaluate damage to the medullary and spleen tissues. For the hematoxylin-eosin technique, the samples were previously washed in the water, processed and embedded in paraffin (Sigma®), and were cut in histological sections of 4 μm. The slides were then de-paraphilized with Xylol I for 20 minutes and Xylol II for 20 minutes at room temperature. Then the hydration step was carried out, which consists of the use of absolute alcohol initially for 2 minutes and ethyl alcohol in 95%, 80% and 70% concentrations for 2 minutes each. The slides were washed in running water and distilled water and then submerged in Harris Hematoxylin (Merck®) for 2 minutes by washing in running water and washing in distilled water for 15 seconds each. The slides then underwent a dehydration process with ethyl alcohol at concentrations of 50%, 80%, 95% for 2 minutes each. Soon after, they were submerged in the eosin for 30 seconds, followed by 95% alcohol and 95% alcohol II for 2 minutes each, and by the absolute alcohol battery I, II and III. After this, the slides were diaphanized with Xylol I, II and III for 2 minutes each. For finalization, the slides were sealed with Permount Mounting Medium (VWR Laboratories, Radnor, Pa., USA) and visualized with the Nikon DS-Ri1 optical microscope provided.

Immunohistochemistry

To perform the immunohistochemical technique, the samples fixed in 10% paraformaldehyde and washed in running water, processed and included in paraffin, to obtain histological sections. The slides were then de-paraphilized with Xylol I for 20 minutes and Xylol II for 20 minutes at room temperature. Then the hydration step was carried out, which consists in the use of ethyl alcohol in 100% and 95% concentrations for 4 minutes each. The slides were then submerged in Ammonium Hydroxide (10% in 95% ethyl alcohol) for 10 minutes, followed by four washes with distilled water for 5 minutes. The slides were subjected to antigenic recovery in citrate buffer for 5 minutes at 95° C. in a pressure cooker, followed by three washes in distilled water for 5 minutes. Next, 10-minute baths were carried out in hydrogen peroxide 6% methanol in 1:1 ratio, for the blockade of the endogenous peroxidase, followed by two washes in distilled water for 5 minutes.

After this step the slides were incubated with the primary antibodies described in Table 1, in a moist chamber overnight at 4° C. The next day, the slides were washed in PBS for 5 minutes. Then, the secondary antibodies described in Table 2 were incubated for 1 hour in a humid chamber and at room temperature. After, two washes in PBS were performed for 5 minutes for removal of the secondary antibody and 100 μL of chromogen (DAB Kit) Zymed® (Zymed Laboratories, San Francisco, Calif., USA) was pipetted in the cuts of 3 to 5 minutes. The slides were washed in distilled water for 5 minutes. They were then submerged in Mayer's Hematoxylin (Merck®) for 1 minute and washed in running water for 15 seconds and washed in distilled water for 5 minutes. The slides then underwent a dehydration process in an up-stack of ethyl alcohol at concentrations of 50%, 80%, 95% for 2 minutes each, and 100% for 6 minutes. After this, the slides were diaphanized with Xylol I, II and III for 2 minutes each. For finalization, the slides were sealed with Permount Mounting Medium and visualized with the Nikon DS-Ri1 optical microscope.

Immunocytofluorescence

BM cells were grown under glass coverslips at 2×10⁴ concentration, and after reaching 50-60% confluency, were fixed in 4% paraformaldehyde solution (Sigma). To remove the paraformaldehyde the cells were washed 2 times with PBS solution for 5 minutes followed by the addition of 0.1% Triton (Sigma®) for 20 minutes to permeabilize the cell membrane. The cells were then washed 2 times with PBS solution for 5 minutes and 5% BSA (Sigma®) added for 40 minutes. Thereupon, primary antibodies described in Table 1 were added and incubated at 4° C. overnight.

Then the cells were washed twice with PBS solution for 5 minutes for the removal of primary antibodies. Then, the secondary antibodies described in Table 2 were added for one hour at room temperature. The coverslips were removed from the culture plates and washed with the vectashield kit with DAPI Vector® (Vector Laboratories, Ltd., Burlingame, Calif., USA) for analysis on the NIKON DS-Ri1 fluorescence microscope (with HBO lamp excitation light for the fluorochrome DAPI, excitation laser 488 nm for FITC).

Cytospin

Aliquots of medullary aspirate were separated at the concentration of 5×10⁴ cells. Samples were fixed at 4% paraformaldehyde (Sigma®) for 24 hours. Then, they were washed 1× with PBS and centrifuged at 2000 RPM for 5 minutes. The supernatants were then discarded, and the pellet of the samples was resuspended in 300 μL of 5% BSA (Sigma®).

Subsequently, silanized slides were mounted in the cytospin apparatus, and 300 μL of sample was pipetted. They were then centrifuged at 2000 RPM for 5 minutes in the specialized Thermo Shandon Cytospin® 4.

Immunocytochemistry

The slides with 5×10⁴ BM cells (previously concentrated by the cytospin technique) were washed twice in PBS for 5 minutes. Next, 0.1% Triton X-100 (Sigma®) was added for 20 minutes to provide cell permeabilization. The cells were again washed in PBS for 5 minutes. After this, the cells were incubated with 5% BSA solution (Sigma®) to block non-specific binding (for 40 minutes). The samples were then washed 1×PBS for 5 minutes and incubated with the primary antibodies described in Table 1, overnight at 4° C. The next day, the slides were washed in PBS for 5 minutes. Then, the secondary antibodies described in Table 2 were incubated for 1 hour in a humid chamber and at room temperature. Thereafter, two washes in PBS for 5 minutes for the removal of the secondary antibody were performed and 100 μL of chromogen (DAB Kit, Zymed Laboratories) pipetted onto the slides for 3 to 5 minutes. The slides were washed in distilled water for 5 minutes. They were then submerged in Mayer's Hematoxylin (Merck®) for 1 minute and washed in running water for 15 seconds and washed in distilled water for 5 minutes. For finalization, the slides were closed with aqueous Abcam® mounting medium (Abcam Plc., Cambridge, UK) and enamel, and visualized with the aid of the Nikon DS-Ri1 optical microscope.

Analysis of Results

After the experiments were carried out, the statistical analysis of the results by ANOVA, two way (ANOVA) were performed using GraphPad Prism 5.0 software (GraphPad; PRISM, 2007).

### Example 2. AA Model and Experimental Design

To induce AA, 40 female isogenic mice of the C57 black/6 line, 4 weeks of age, received whole body irradiation in a panoramic source of radiation containing Cobalt 60, with a mean rate of 11.60 kg and 40 cm of distance from the source. The dose used was 6 Gy, since it is considered moderate to severe. The exposure time of the animals in the irradiation chamber was 31 minutes. Each cage contained three animals, with food and water served ad libitum. Light cycles were maintained, and the temperature was maintained at 19-22° C. This study was approved by the Committee on Ethics in the Use of Animals of the Butantan Institute. Process number 01201/14.

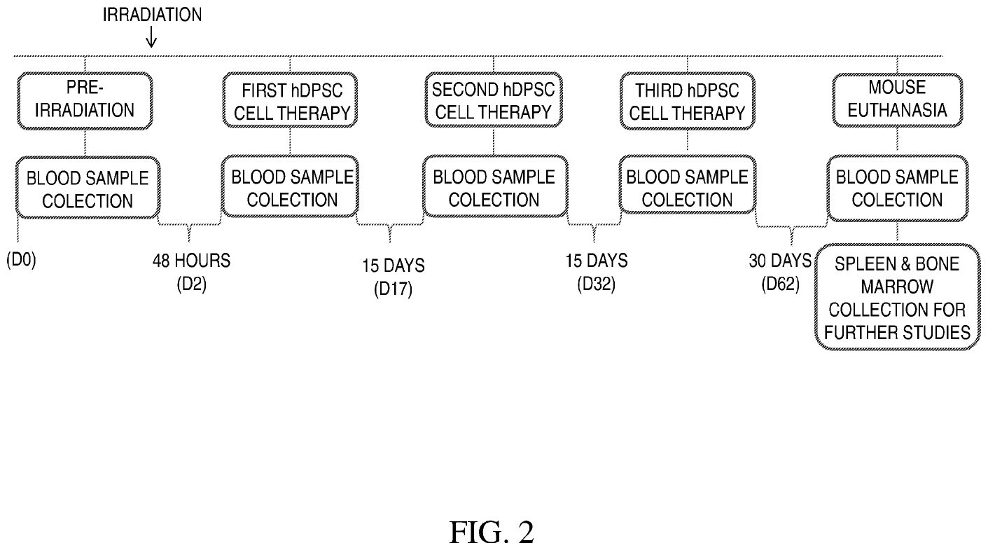

The irradiated mice after 48 hours of irradiation were divided into 2 groups (FIG. 2, Table 3):
Group treated with IDPSCs: irradiated animals+transplantation with IDPSCs via intraperitoneal using 1×10⁶ cells. N=20 animals;
Group not treated with IDPSCs: irradiated animals+PBS solution. N=20.

The animals received the first transplant of 1×10⁶ IDPSC after 48 hours of the irradiation process, the second transplant was performed 15 days after the first transplant, and the third transplant was performed 15 days after the second transplant, totaling three cell transplants (FIG. 2, Table 3).

TABLE 3
Experimental design

| Groups | Treatment | No. |
|---|---|---|
| Control | Non-irradiated (physiologic normal) | 10 |
| Irradiated | Irradiated and euthanatized after 48 hours | 10 |
| treatment | Irradiated + intraperitoneal injection with | 40 |
| | hIDPSC cells (1 × 10⁶) | |
| Non-treatment | Irradiated + intraperitoneal injection with | 40 |
| | saline solution | |
| Long term | Irradiated + intraperitoneal injection with | 5 |
| treatment | hIDPSC cells (1 × 10⁶) + euthanatized after | |
| | 6 months | |
| Long term | Irradiated + intraperitoneal injection with | 5 |
| non-treatment | saline solution (1 × 10 ⁶) + euthanatized after | |
| | 6 months | |

The animals were treated via intraperitoneal (IP) injection. A great advantage of using the IP pathway is the possibility of administering a large number of cells because peritoneum is capable of high absorption due to its high vascularization.

For the IP transplant, 1×10⁶ IDPSCs were resuspended in 100 μL of saline solution and then injected into the abdominopelvic cavity with an insulin syringe. The animals were followed up for 30 days after the date of the last cell transplantation. At this time, the animals were euthanized in a CO₂ chamber to collect the long bones (femur and humerus) and the spleen.

The hemograms were performed at the following time points: before irradiation (D0), after irradiation (D2), 15 days after each transplant (D17 and D32), and 30 days after the third transplant (D62) (FIG. 2). Control of the body mass of the animals was performed in the same chronological interval.

Six animals that were not irradiated for control of the study were separated, and six animals that underwent irradiation were euthanized 48 hours after irradiation to prove the efficiency of the radiation as a model of AA induction.
Control Group (A): Non-irradiated animals (wild type) N=6 animals;

Control Group (B): Non-irradiated animas treated with IDPSCs=6 animals;
Irradiated group: Animals irradiated and euthanized 48 hours after irradiation, N=6 animals.

Additional studies were carried out to evaluate the persistence of the treatment; therefore, a long-term group was included in the experimental design. In this group, a portion of the experimental animals (n=10) were not euthanized after the period proposed in this study (D62). These animals remained a period of 6 months after D62.
Group 6 months post-D62 Treated (irradiated+treated with IDPSCs+6 months), N=5 animals;
Group 6 months post-D62 Untreated (irradiated+without treatment+6 months), N=5 animals.

### Example 3. Expression of Selective Markers Involved in Hematopoiesis by IDPSCs

Passage P4 or P5 IDPSCs were used. The immunophenotypic characteristics of IDPSCs described previously (KERKIS et al., 2006; LIZIER et al., 2012) were confirmed in our present study (data not shown).

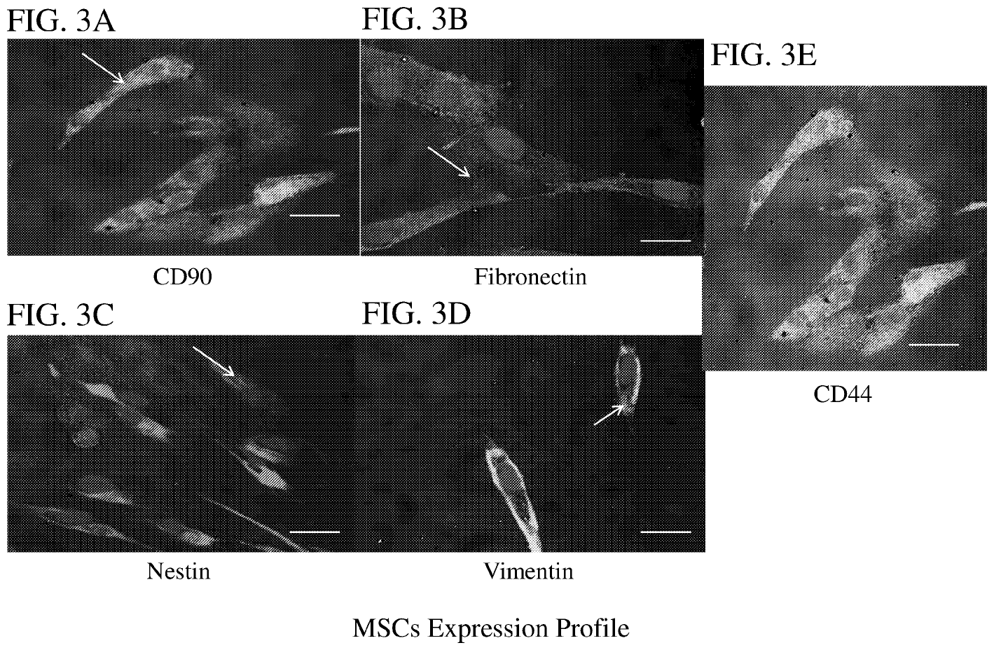

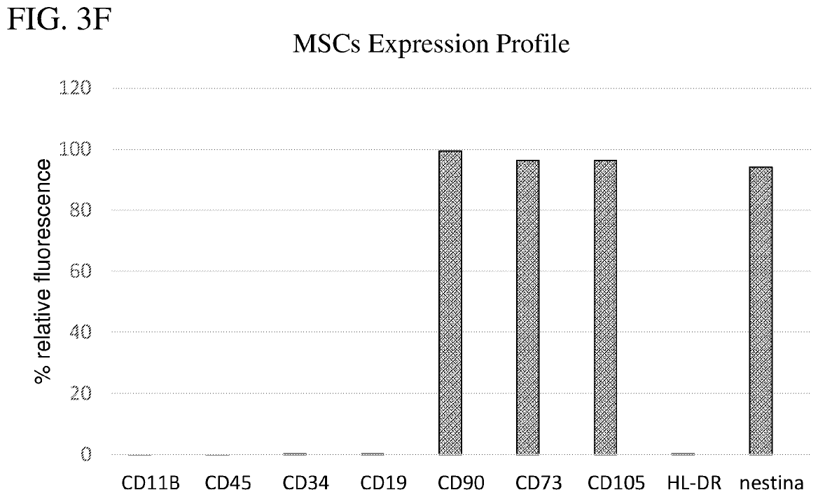

Immunocytofluorescence assays were performed using antibodies against CD90, Fibronectin, Nestin, Vimentin, or CD44. All these markers were immunopositive in IDPSCs. Immunostaining of CD90 was detected on the cell membrane by FITC (white arrow) (FIG. 3A). Immunostainings of Fibronectin, Nestin, Vimentin, and CD44 were detected in the cytoplasm by FITC (white arrow) (FIGS. 3A-3E).

FIG. 3F Flow cytometry analysis demonstrates expression of additional MSC markers that are positive in IDPSC: CD90⁺; CD73⁺, CD105⁺ and Nestin⁺. The expression of these markers and other that presented on FIG. 3 observed in between about 80% and 100% of cells. FIG. 3F also demonstrates that these cells are negative for CD11B; CD45; CD34; CD19 and HLA-DR. Overall, we can describe immuno profile of these cells as CD90⁺; CD73⁺, CD105⁺, Nestin⁺, CD44⁺, Vimentin⁺, Fibronectin⁺/CD11B⁻; CD45⁻; CD34⁻; CD19⁻, HLA-DR⁻.

### Example 4. Body Weight

The body weight of the animals was evaluated before irradiation (D0), 48 hours after irradiation (D2), and 15 days after each transplant with IDPSCs, first transplant (D17); second transplant (D32), and 30 days after the third transplant (D62/euthanasia).

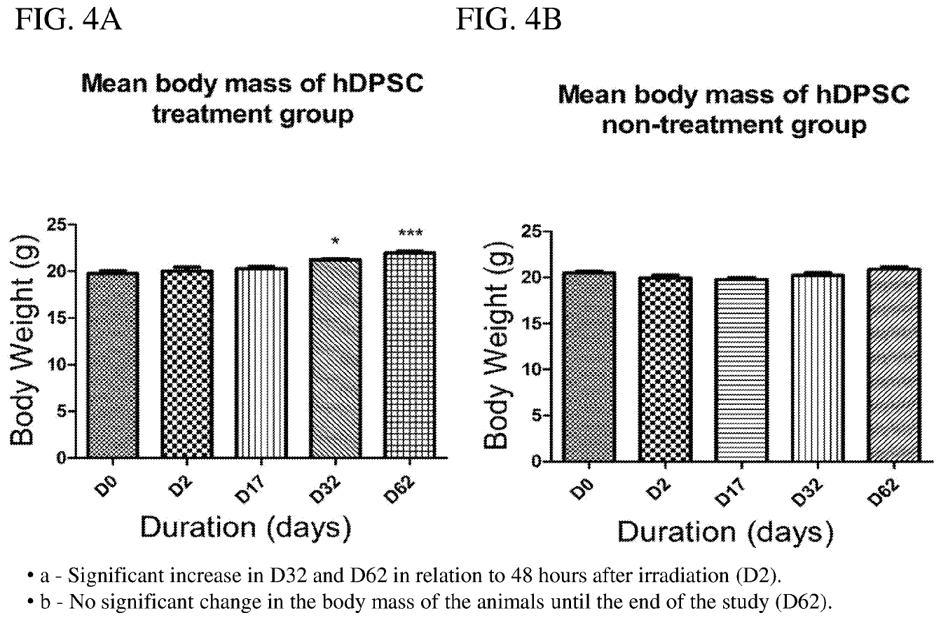

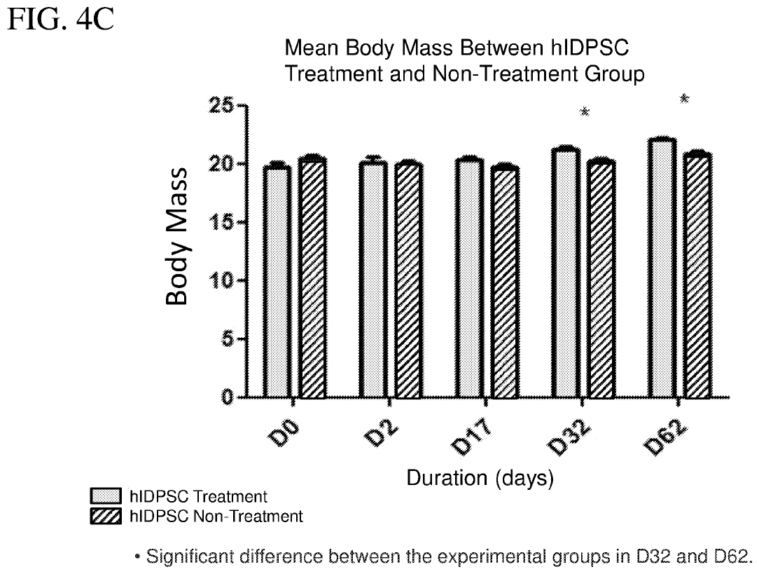

Mice from both experimental groups showed no significant decrease in body mass 48 hours after irradiation (6 Gy) (D2). However, the animals that received the 2nd IDPSC transplant (D32) showed a significant gain of body weight (P<0.05) in relation to D2 (48 hours after irradiation). This gain was more significant after the third transplant (D62) (P<0.001) (FIG. 4C). Meanwhile, animals from the untreated group (irradiation+saline) showed no significant change in their body mass during treatment (FIGS. 4A-4B).

### Example 5. Monitoring of Hematimetric Parameters

Erythrocytes

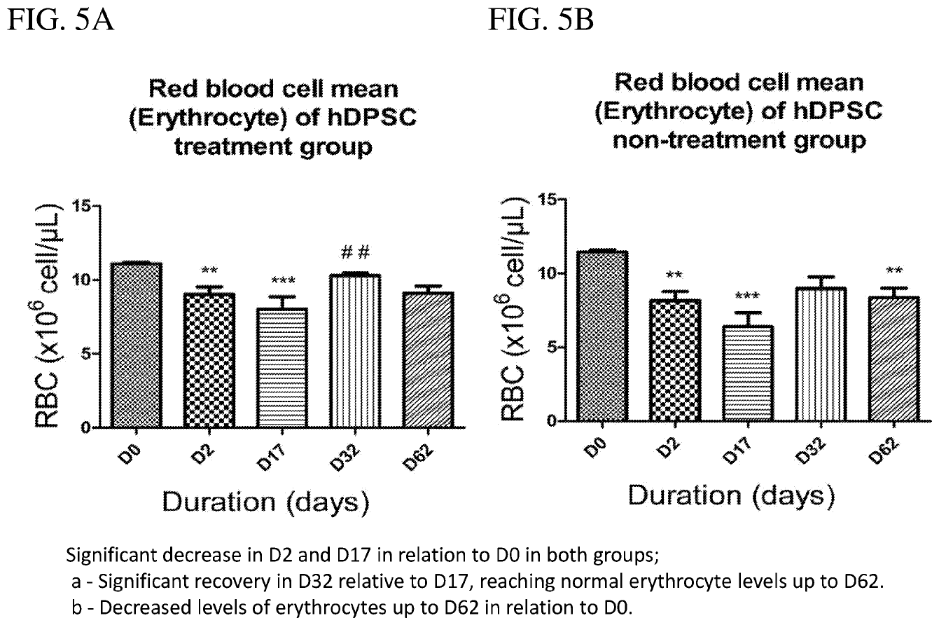

The hemogram showed a significant decrease in the number of erythrocytes after irradiation (D2) in both experimental groups (treated with IDPSCs and untreated) (P<0.01), which reached values below the reference of normal mice (>11×10⁶/μL). These data show the efficacy of irradiation at the dose of 6 Gy. It is worth mentioning that there was a more pronounced decrease in erythrocyte values in D17 (P<0.001) in both experimental groups (treated with IDPSCs and untreated) (FIGS. 5A-5E). However, in D32 animals treated with IDPSCs showed a significant increase (P<0.05) in absolute erythrocyte numbers in relation to the drop indicated in D17 (represented by ##), reaching normal reference values. This profile was observed until the end of the study period (D62). While the absolute number of erythrocytes of the animals in the untreated group remained below the reference values up to D62 (P<0.01) (FIGS. 5A-5B).

Comparison between groups shows that animals receiving IDPSCs treatment had erythrocyte levels higher than the group that did not receive the treatment, which was not even able to reach the normal reference values (>11×10⁶/μL) until the end of the study.

IDPSCs were also transplanted into wild-type animals (Control group B), which did not receive irradiation. Comparison between Control groups A and B did not demonstrate any difference in erythrocyte levels (FIGS. 5C-5E). Reference values for hemogram (Mouse): Erythrocytes (RBC=7-11×10⁶/μL).

Hematocrit Values

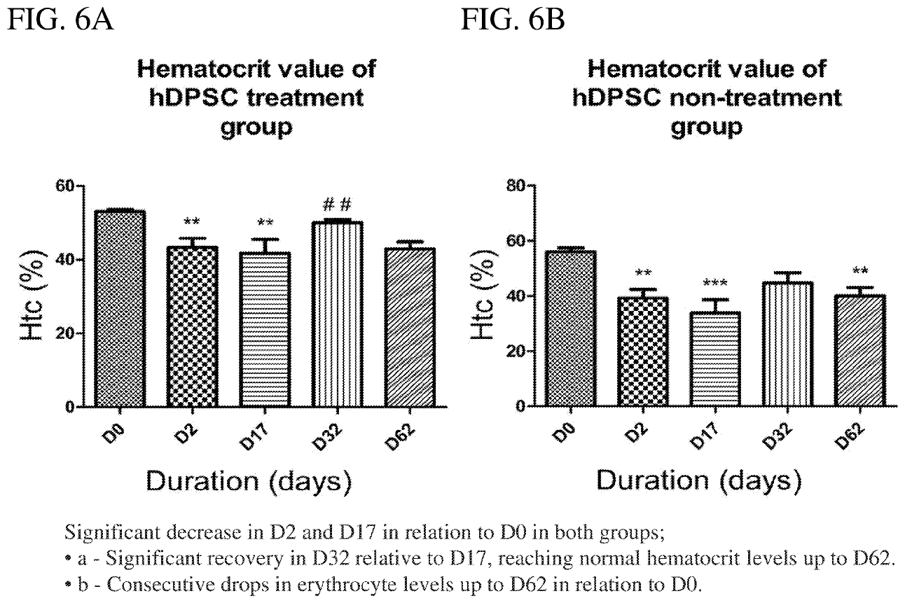

The results showed a significant decay in hematocrit values in both experimental groups shortly after irradiation (D2) (P<0.01). This decay continued in D17 (P<0.001) in relation to D0, reaching values below 50%, considered lower than the normal reference level for mice (WEISS; WARDROP, 2010) (FIGS. 6A-6B). However, in D32, the IDPSC treatment group showed a significant increase in the percentage of hematocrit (P<0.01) (represented by ##) in relation to D17, recovering the normal values for the species, which remained to D62. While the group not treated with IDPSCs did not recover normal hematocrit levels up to D62 (P<0.01) (FIGS. 6A-6B).

When comparing the hematocrit values between the IDPSC treated and untreated groups, it was possible to observe that the animals that received the treatment were able to normalize the hematocrit levels after the second transplant (D32). They showed the percentage of hematocrit higher than untreated animals, which did not recover the normal hematocrit value according to the reference by the end of this study (>50%).

IDPSCs were transplanted into wild-type animals (Control group B), which did not receive irradiation. Comparison between Control groups A and B did not demonstrate any difference in hematocrit percentage (FIG. 5D). Reference values for hemogram (Mouse): Erythrocytes (RBC=7-11×10⁶/μL)

Leukocytes

The absolute values of leukocytes in both experimental groups (treated with IDPSCs and untreated) had a significant reduction after irradiation (D2) (P<0.001). The decrease in absolute values of leucocytes remained at all points analyzed until the end of the study (D17, D32, and D62) in both experimental groups (P<0.001). These results demonstrate that leukocyte levels had a greater reduction compared to erythrocyte and hematocrit levels. This may be explained because the immune system responds quickly, and is very sensitive to whole-body irradiation, largely because of the tendency of lymphocytes to undergo apoptosis. In fact, the rate of decrease in the number of lymphocytes in the peripheral blood occurs on the first days after exposure to radiation. Another important factor to consider is that the peripheral blood has a high number of circulating lymphocytes, so a reduction in the number of this lineage is more significant. In addition, the hematopoietic system is extremely sensitive to ionizing radiation.

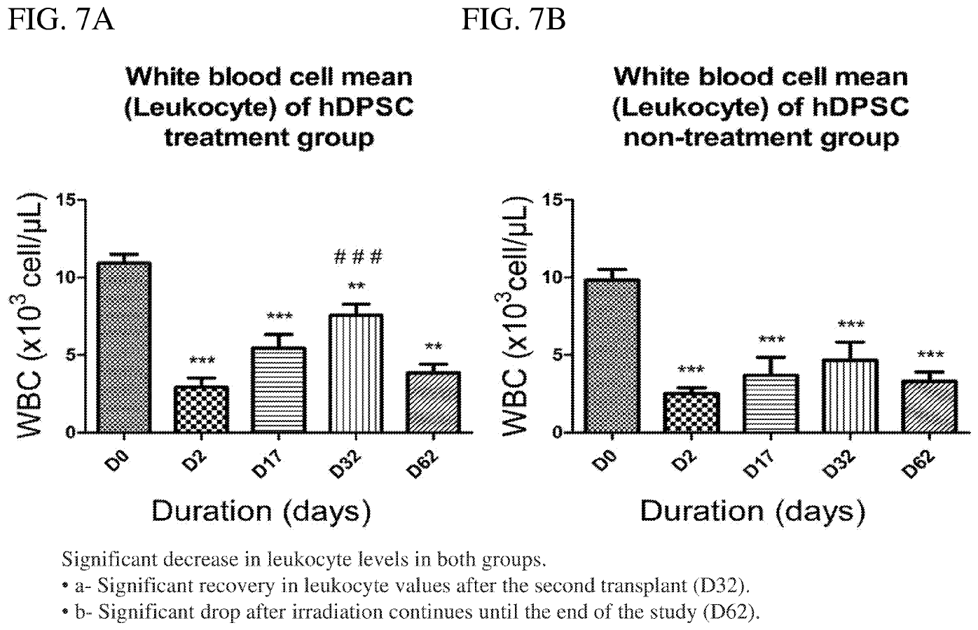

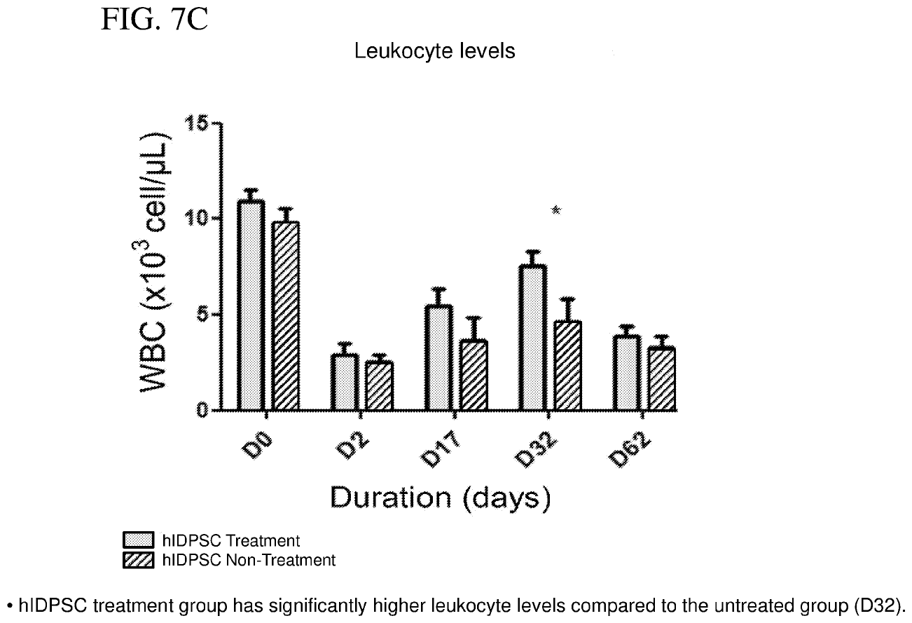

Only the IDPSC-treated group showed a slight recovery in total leukocyte levels after the second cell transplantation (D32) (P<0.001) (represented by ###) on D2. However, untreated animals showed no significant variation in the absolute values of leukocytes on D17, D32, and D62 (FIGS. 7A-7C).

Although leukocytes showed a significant decrease during the study period in both experimental groups until the end of the study (D62), there was an increase in leukocyte levels in the IDPSC group, although it did not reach normal levels for the species, while this was not observed in untreated animals.

When comparing total leukocyte values between the experimental groups, a higher leukocyte count was observed after the first IDPSC transplantation, which is significant after the second cell transplantation (P<0.05) (FIGS. 7A-7B).

IDPSCs were transplanted into wild-type animals (Control group B), which did not receive irradiation. Comparison between Control groups A and B showed a slight difference in Leucocytes level (FIG. 5E). However, these values do not exceed reference values established for hemogram (Mouse): Leucocytes (WBC=2-10×10³/μL).

### Example 6. Morphological Changes Observed in Bone Marrow Before and after Irradiation and IDPSC Transplant

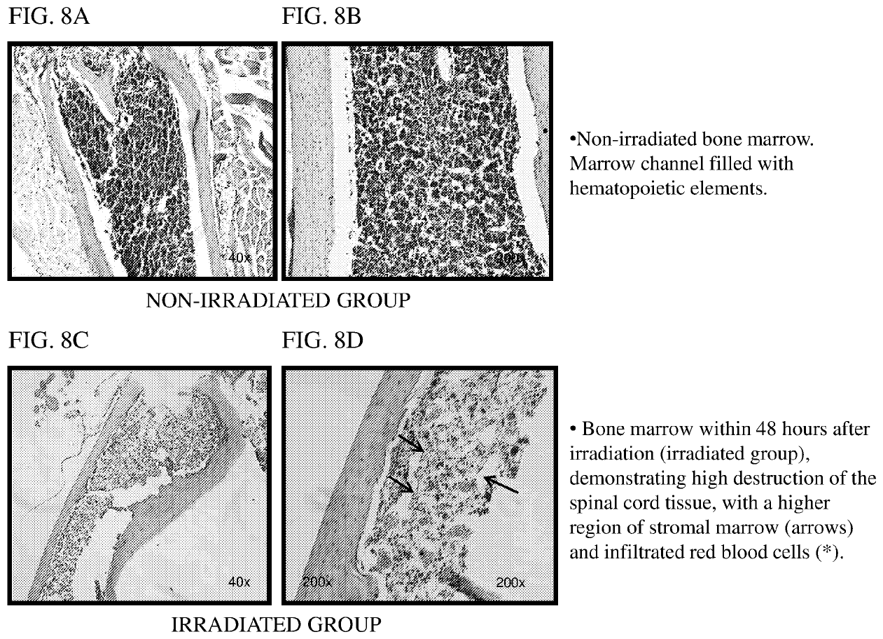

After irradiation (48 hours), bone marrow presented tissue destruction, with disseminated lesion along the medullary tissue and reduction of cellularity (hypoplasia/medullary aplasia), compared to BM of a normal animal (without irradiation). We identified a few hematopoietic elements. These include an absence of megakaryocytes, an infiltration of red blood cells, a greater amount of spinal stromal, and substitution of fatty elements in some regions of BM (FIGS. 8A-8B). These histological findings are similar to those described in the literature for experimental models of myelosuppression or myeloablation with ionizing radiation at doses starting at 4 Gy. These morphological changes are consequences of the impact caused by the radiation to the tissues, resulting in a tissue injury due to oxidative stress and inflammation, which alter the cellular microenvironment and especially the niches of hematopoietic cells that are more sensitive to radiation damage.

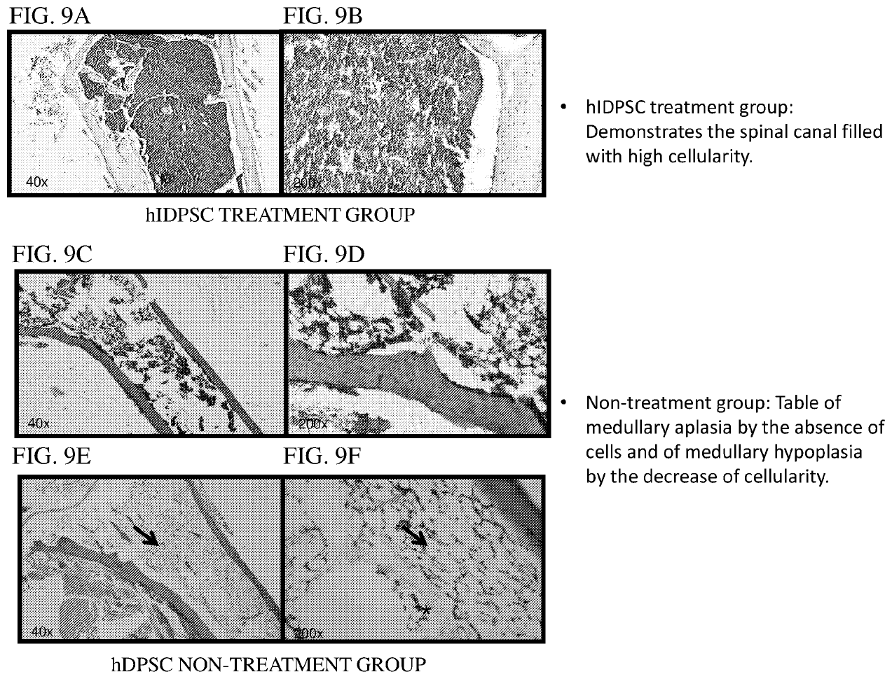

The cellularity of BM in animals treated with IDPSCs was similar to the non-irradiated group, with an absence of adipose cells (FIGS. 9A-9B). The results suggest that IDPSCs can regenerate BM after irradiation. Different results were observed in the animals of the group not treated with IDPSCs. In this group, the BM of the irradiated animals still presented partial or total myeloablation, with reduction of the cellular elements in general, and some regions were marked by the replacement of adipose cells (FIGS. 9C-9E). In some cases, the damage was very severe and was incompatible with life (FIG. 9F).

Bone Marrow Smear Analysis

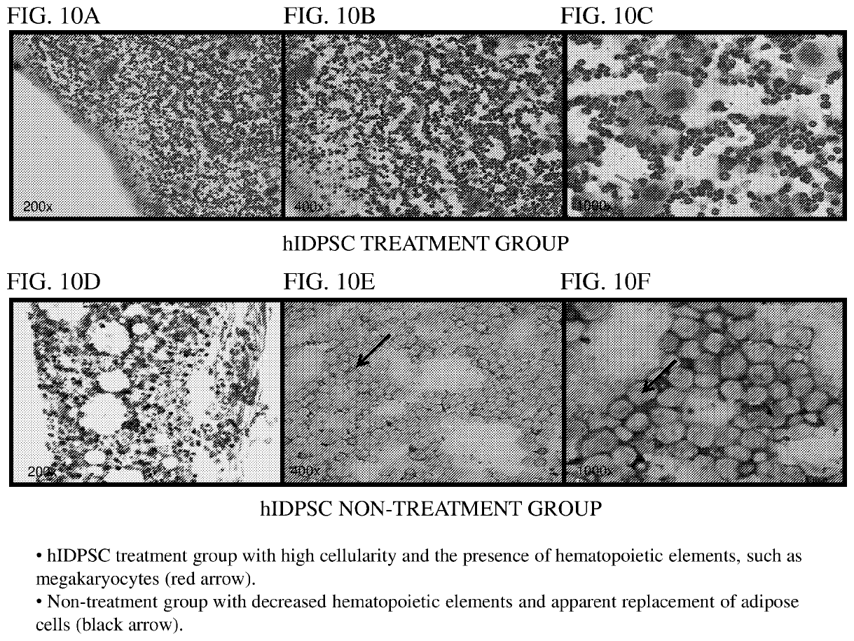

The results obtained after bone marrow smear analysis corroborate with the histological results presented above. On D62 (euthanasia), mice treated with IDPSCs showed higher cellularity, with the presence of hematopoietic precursors and nucleated cells in different degrees of maturation, such as megakaryocytes (FIGS. 10A-10C). While untreated group showed low cellularity, marked by the presence of large numbers of adipocytes, and an absence of hematopoietic precursors and megakaryocytes (FIGS. 10D-10E).

It is known that thrombocytopenia is one of the most important hematimetric indexes for AA that make patients who have this disease exposed to hemorrhage. Also, platelet deficiency can cause bruises (and bone marrow edema or bone contusion), which are blood infiltrations (red blood cells) in the BM due to rupture of capillaries. These infiltrates were found in BM of animals after irradiation and in BM of animals not treated with IDPSCs.

The absence of megakaryocytes in the BM after irradiation and without treatment with IDPSCs can be observed in animals with AA (FIG. 10E). While in the mice treated with IDPSCs, it was possible to visualize the presence of megakaryocytes (FIG. 10C). Therefore, these data provide indirect evidence that treatment with IDPSCs may also induce an improvement in the platelet function.

Together, these results (smear and histology of BM) demonstrate the effectiveness of IDPSCs in the recovery of the cellular components of BM in irradiated mice.

Colony-Forming Unit Fibroblasts

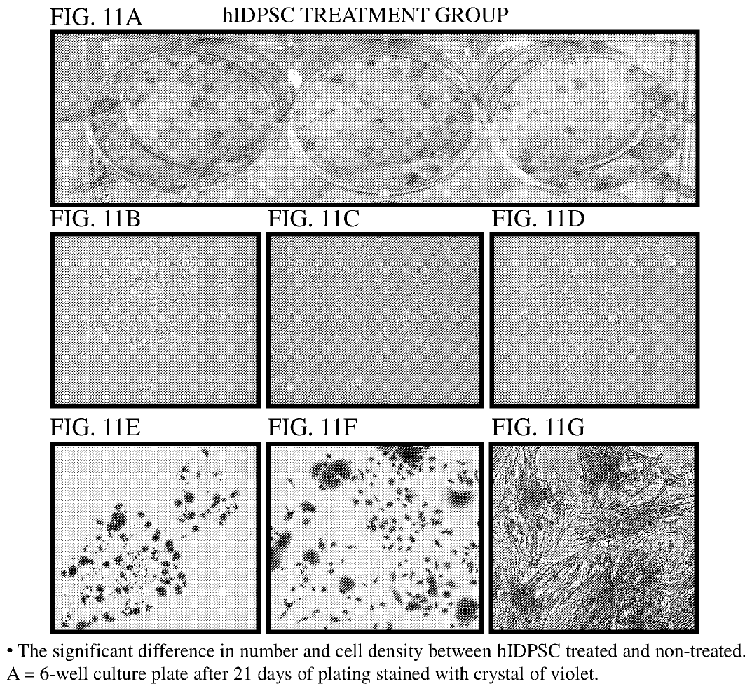

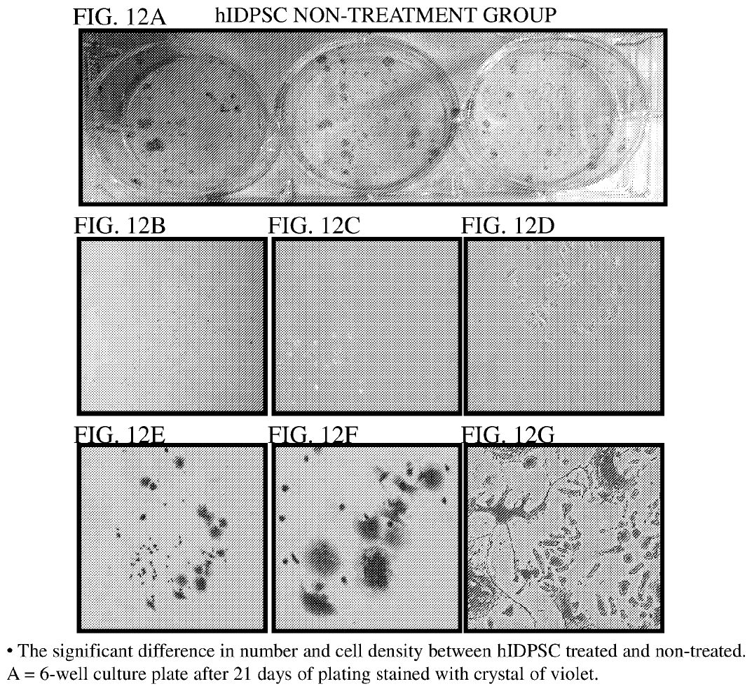

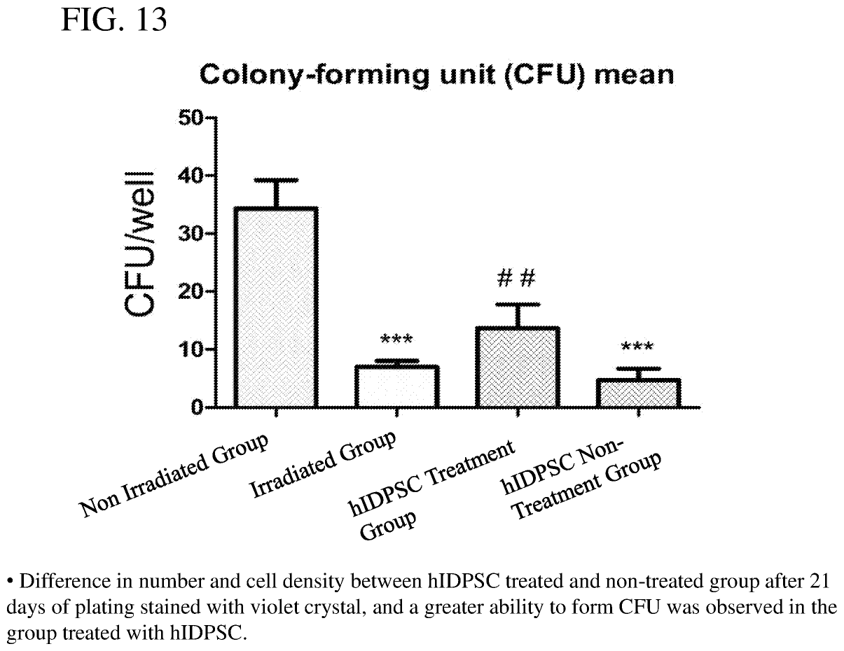

It is known that radiation at the dose of 10 Gy reduces the proliferative capacity of BM cells. Thus we evaluated whether the transplanted IDPSCs may influence the proliferative capacity of the BM cells in the irradiated mice. BM aspirate was collected, and the CFU-F assay was performed. A greater ability to form CFU-F was observed in the group treated with IDPSCs (FIGS. 11A-11G and FIGS. 12A-12G).

The statistical analysis of the number of colonies formed was carried out to determine the influence of IDPSCs in the formation of CFU. It was possible to observe a significant decrease (P<0.001) in the number of colonies in the animals with irradiation (48 hours) compared to the animals without irradiation. This reduction in the clonogenic capacity of BM cells was also significant (P<0.001) in the animals not treated with IDPSCs compared to the control (without irradiation). However, in animals receiving IDPSC treatment, no significant decrease in the number of CFUs was observed compared to the control (FIGS. 12A-12G). Also, a significant difference (P<0.05) (represented by ##) was observed between the experimental groups, where the animals that received IDPSCs showed a higher number of CFUs compared to the untreated group.

Given the above results, we can conclude that irradiation at the dose of 6 Gy affects the formation of CFU-F and that IDPSC transplantation protects the BM cells after irradiation. This is an important result because BM cells have a key role in supporting hematopoiesis.

### Example 7. Evaluation of the Functional Contribution of IDPSCs in the Hematopoietic and Stromal Function of the Bone Marrow in Mice

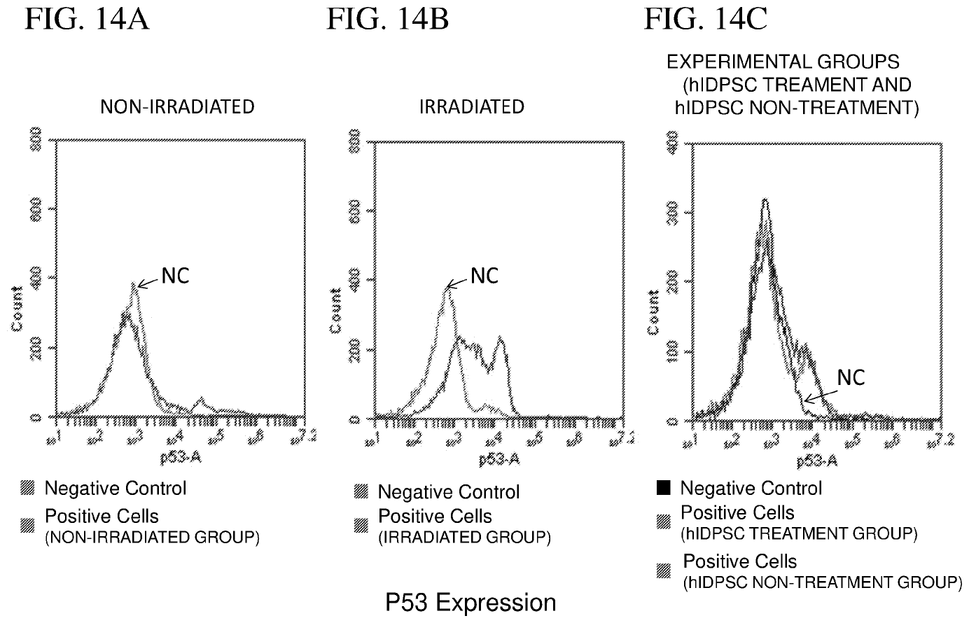

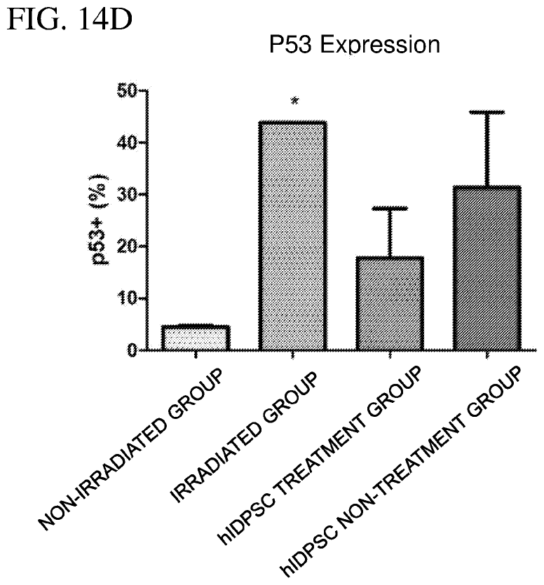

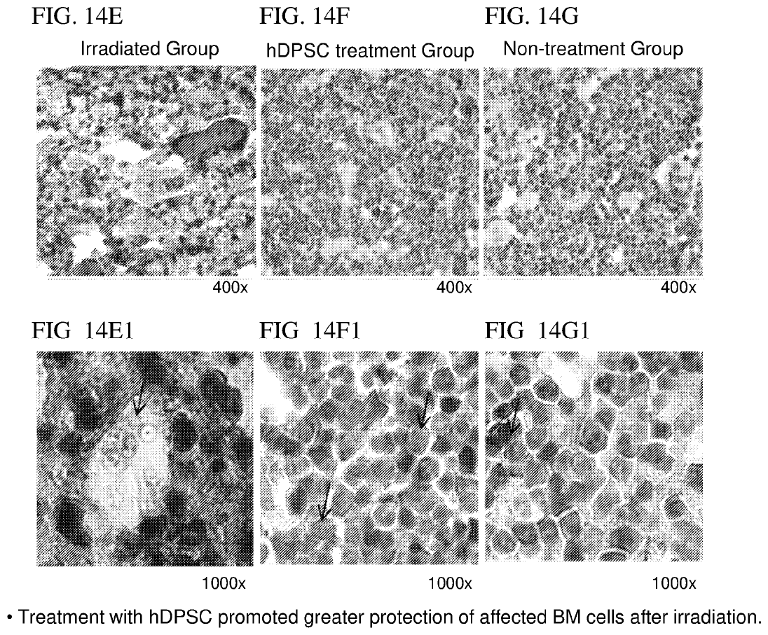

Apoptosis is considered the main cause of BM injury by irradiation. The occurrence of apoptosis is related to the oncogene and anti-oncogene regulation. The cytoplasmic protein p53 is an important biomarker for apoptosis. P53 controls the switch from the G1 to S phase in the cell cycle and thus determines whether the cells will enter the S phase (of the cell cycle) and/or continue for cellular apoptosis. Hematopoietic cells are highly sensitive to radiation damage and even exposure to relatively low levels may result in BMF. The BM irradiation is an important experimental model for study in hematological diseases. Our results demonstrated that irradiation at the dose of 6 Gy resulted in BM cell death by apoptosis. Flow cytometric analysis demonstrated that after irradiation (48 hours), almost 50% of the BM cells were apoptotic (mean 43.8% p53-positive cells). While only 4.46% of the cells in the control group (without radiation) were p53-positive (P<0.05) (FIGS. 14A-14G1).

At the end of this study (D62), the expression of p53 was reduced in both experimental groups. In this same period, the group treated with IDPSCs showed lower number of p53⁺ cells (mean: 17.75%) compared to the untreated group (mean: 31.35%) (FIG. 14D). This finding demonstrates a possible anti-apoptotic effect of IDPSCs on BM cells.

IDPSCs Action on the BM Extracellular Matrix

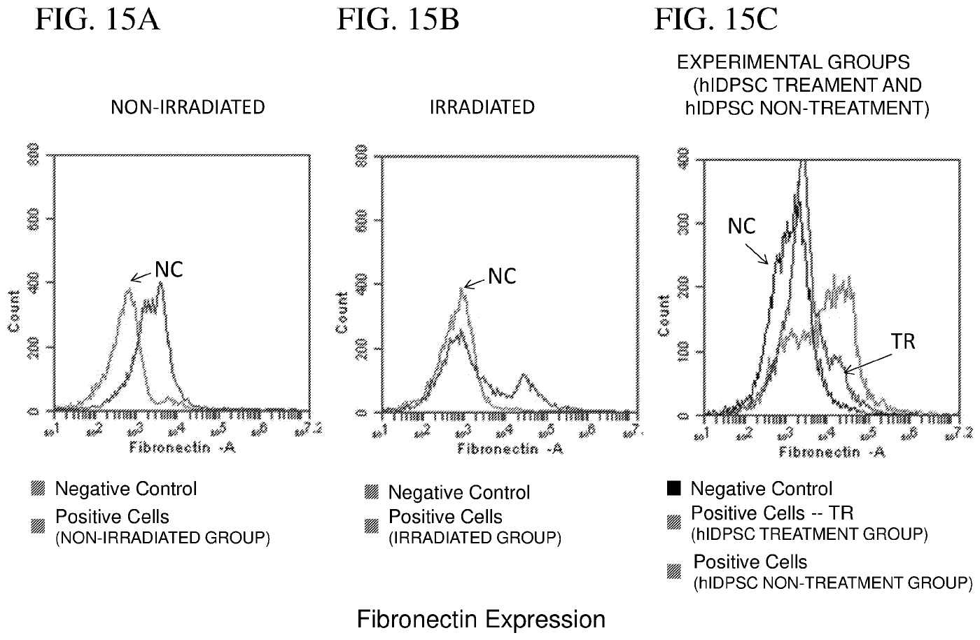

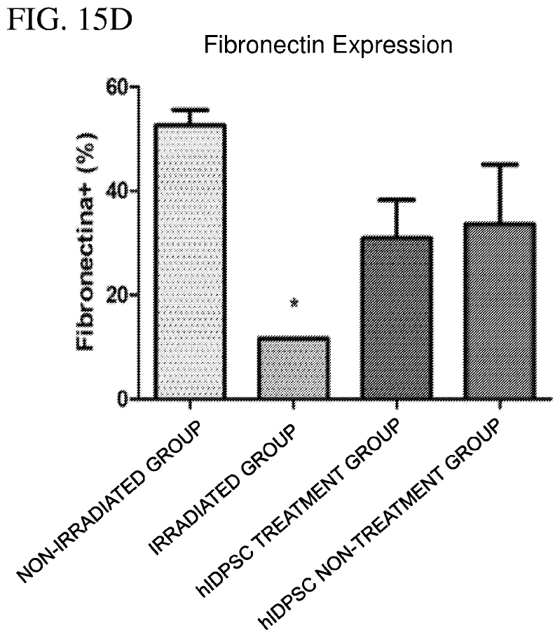

After irradiation at the dose of 6 Gy, a reduction of Fibronectin (P<0.05) (mean: 11.7% Fibronectin-positive cells) was observed in comparison to the animals in the control group (mean: 52.63% of Fibronectin-positive cells). However, we did not observe a significant difference between treated and untreated groups in the percentage of Fibronectin-positive cells (FIGS. 15A-15D).

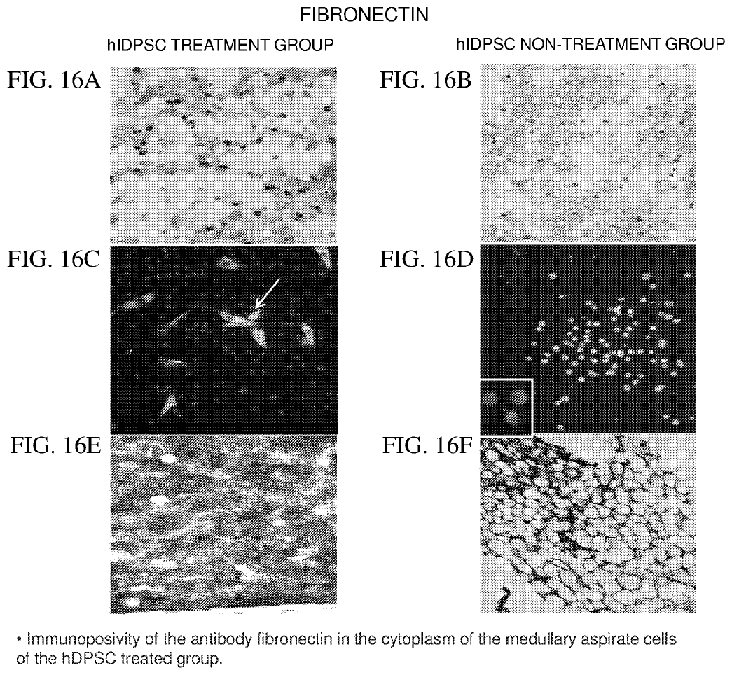

We compared the expression of Fibronectin in IDPSC-treated versus untreated animals using immunocytochemistry, immunocytofluorescence, and immunohistochemical analysis (FIGS. 16A-16F). BM aspirate in the IDPSC-treated group showed fibronectin⁺ cells with characteristic fibroblast-like morphology that is different from those of untreated animals (FIGS. 16C-16D). Fibronectin, an extracellular matrix protein, was observed in both groups but has a distinct pattern of localization in the extracellular matrix (FIGS. 16E-16F). High amount of fibronectin with a characteristic extracellular matrix formation in an extensive network was observed in bone morrow of irradiated and IDPSC treated mice (FIG. 16E). In untreated group rude fibers of fibronectin between adipose cells were observed (FIG. 16F). These findings suggest a role for IDPSCs in matrix remodeling.

DPSC Action in Vascular Niche

Nestin

Another BM component, the vascular niche, is composed of endothelial cells, endothelial progenitors, and MSC. Nestin, an intermediate filament in neural progenitors, is also expressed in proliferative endothelial progenitors and have a fundamental role in angiogenesis. Also, Nestin is widely expressed in perivascular cells, MSCs, and IDPSCs that are derived from the neural crest. Nestin can be used as a vascular niche biomarker (endothelial progenitors).

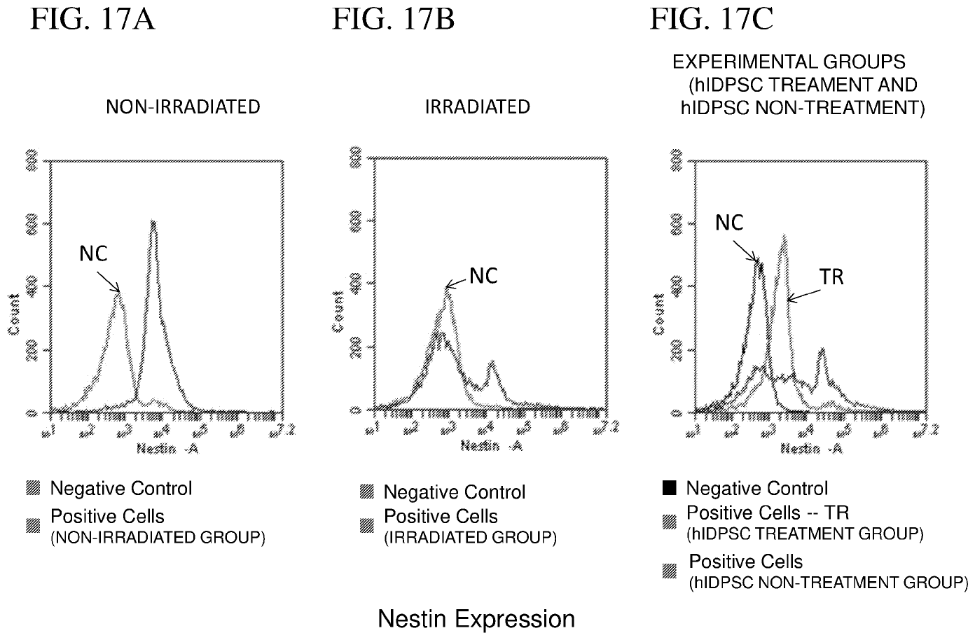

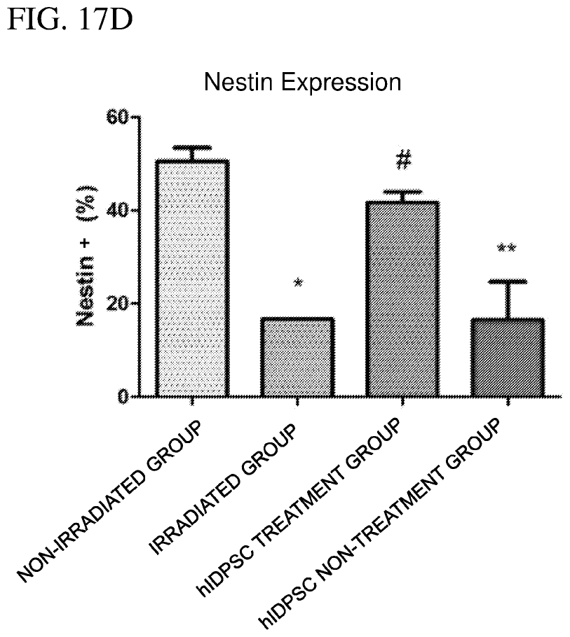

The results obtained by the flow cytometric analysis showed that the radiation caused a significant decrease in the number of Nestin-positive cells (P<0.05) (mean: 16.6% Nestin-positive cells) compared to the control group (not irradiated) (mean: 50.53% Nestin-positive cells) (FIGS. 17A-17D). The decrease of endothelial precursors has also been observed in patients with AA who have compromised endothelial vessels due to the disease. After transplantation with IDPSCs (D62), it was observed that the number of Nestin-positive cells increased significantly (P<0.05) in the group treated with IDPSCs (mean: 41.6% Nestin-positive cells) compared to the untreated group. Interestingly, the group that did not receive treatment show a significant decrease (P<0.05**) (mean: 16.4% Nestin-positive cells) until the end of the study (FIG. 17D).

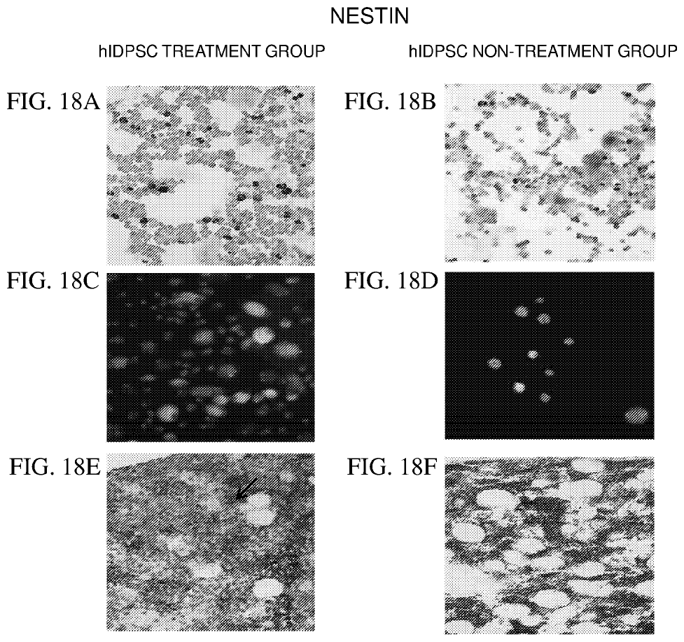

Also, high expression of Nestin in the endothelium of the sinusoid vessels was observed from the group treated with IDPSCs using immunohistochemistry. No such labeling besides a decrease of sinusoidal vessels of the studied area was observed in the untreated group (FIGS. 18A-18F).

CD105

Similar results were obtained with CD105 which is also expressed in endothelial progenitors, endothelial cells, as well as in stromal cells (MSCs).

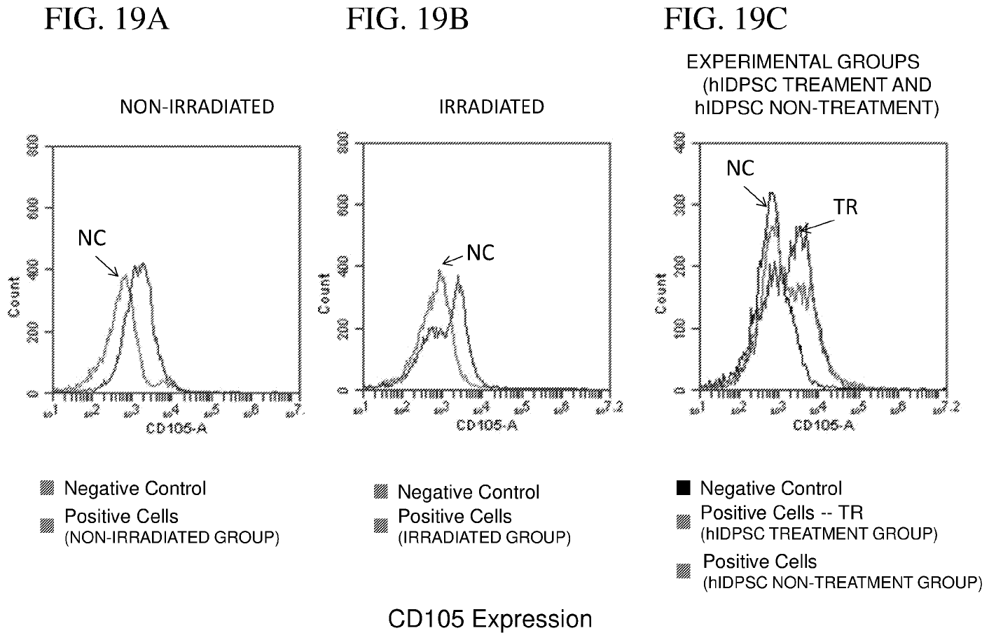

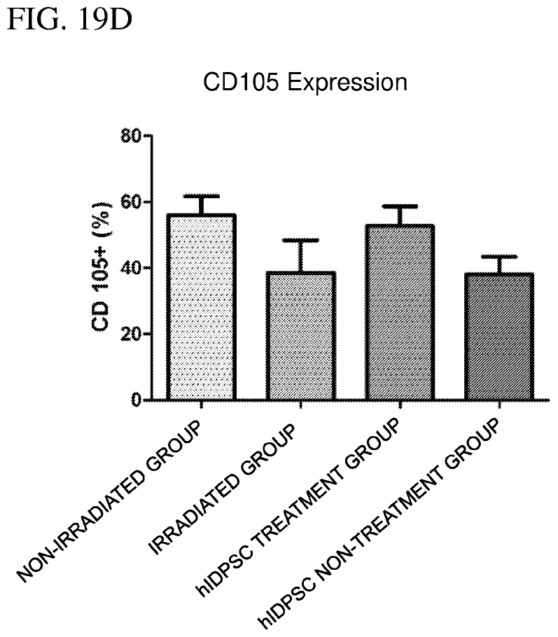

Irradiation (48 hours) caused a decrease of CD105 expression in relation to the control group. These results corroborate with those reported for patients with AA who have compromised endothelial vessels due to the disease. IDPSC treatment apparently recovered CD105 expression. While a slight reduction in CD105 expression remained in the untreated group until the end of the study (FIGS. 19A-19D).

Given that medullar microenvironment and angiogenesis are impaired in AA, leading to decreased of endothelial cells and their progenitors, the increase of Nestin-positive and CD105-positive proliferative endothelial progenitor cells is important for the protection of these cells and the improvement of angiogenesis.

IDPSC Action in Hematopoietic Niche

CD34, C-Kit and Sca-1

One of the benefits of MSCs on hematopoiesis is their role in the secretion of several growth factors and cytokines that support the growth and differentiation of the hematopoietic lineage. Different studies demonstrate that MSCs stimulate HTCs in vivo and in vitro.

MSCs secrete a large number of interleukins that act directly on hematopoiesis, such as IL-6, IL-7, IL-8, IL-11, IL-14, IL-15, Flt-3 ligand, and stem cell factor. However, after irradiation, there is a significant reduction of HTCs and MSCs in BM, which consequently affects the production of the whole blood line. Mouse HSCs express the hematopoietic stem cells markers such as Sca-1, C-kit, and CD34. To verify the mechanisms of action of IDPSCs in HTCs and their progenitors, we used CD44, CD45, CD34, C-kit and Sca-1 antibodies in flow cytometry quantification assays and immunohistochemistry and immunocytochemistry.

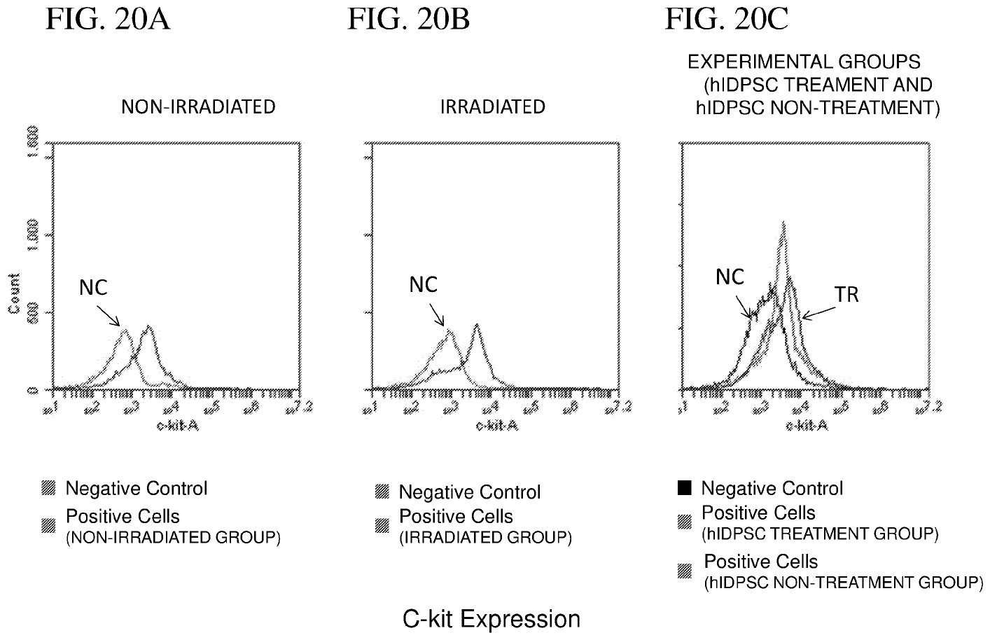

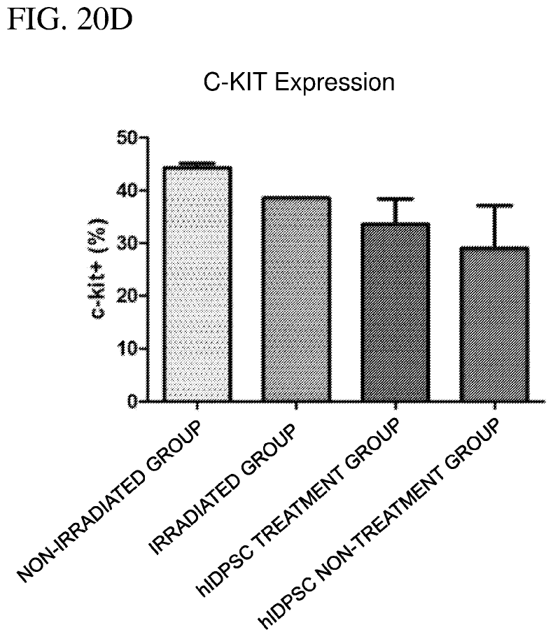

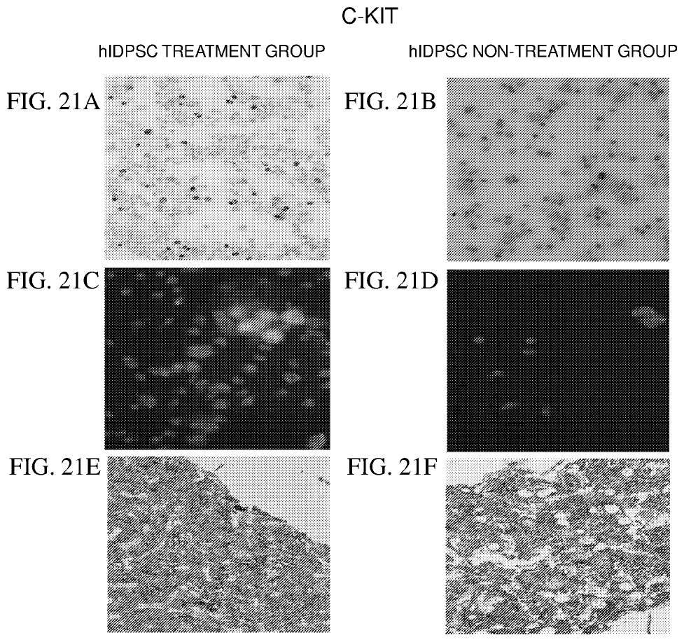

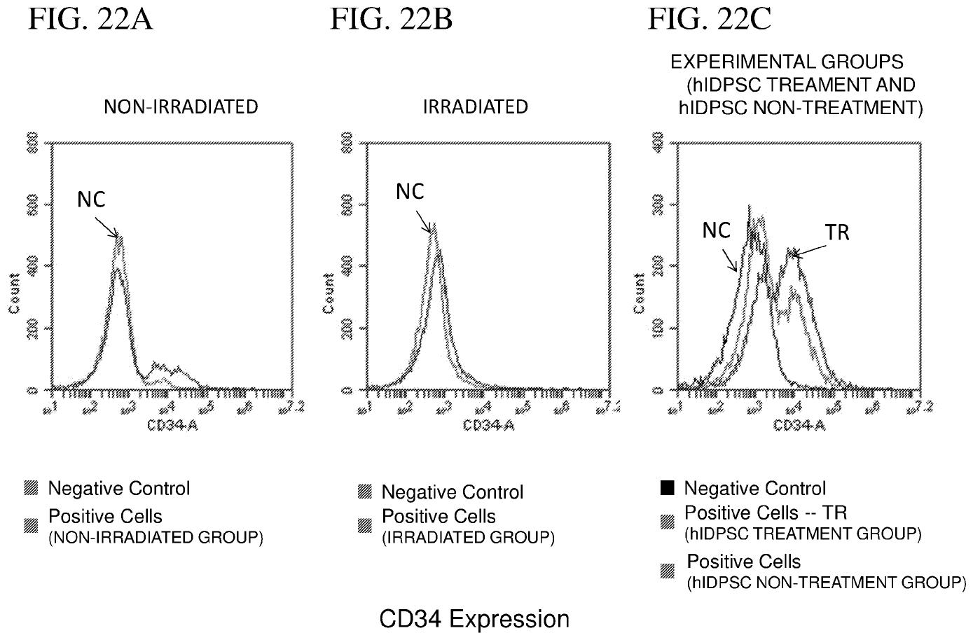

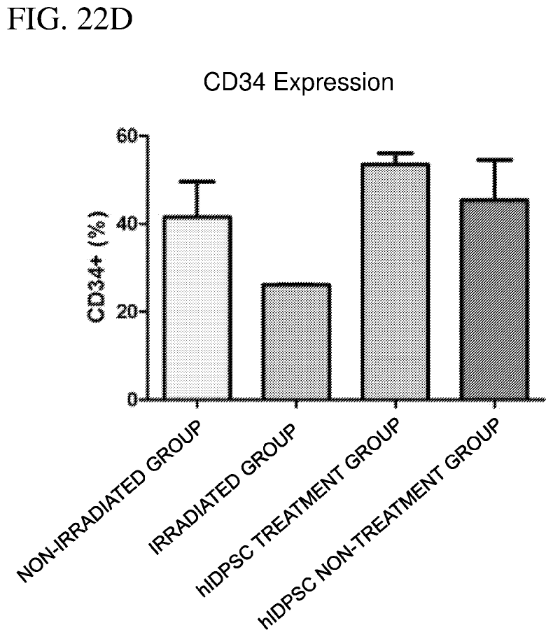

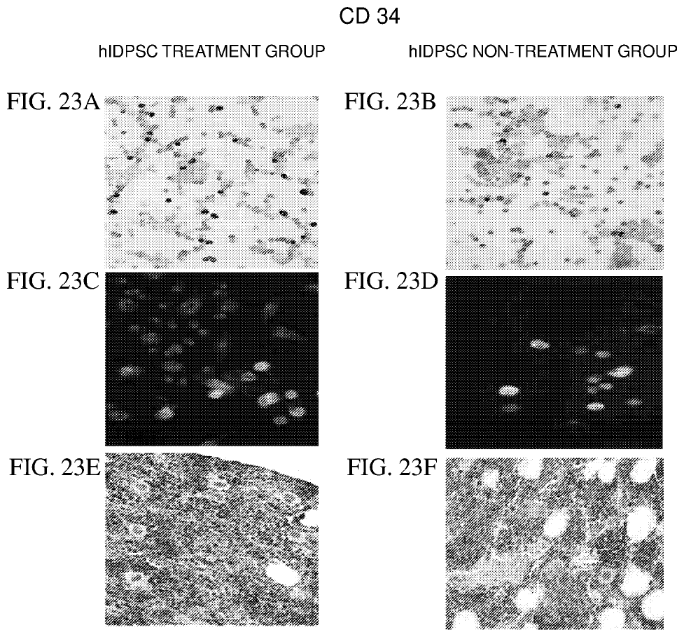

Despite being an important marker of the HSCs, C-kit is also a marker of precursors, multipotent progenitors, and progenitors of compromised lineages. C-kit is expressed in several BM cell types and exerts a broad spectrum of action. We observed a difference between the experimental groups (treated and untreated with IDPSCs). The C-kit expression is slightly higher in untreated group compared to the IDPSC treated group (FIGS. 20A-20D and 21A-21F). Radiation at the dose of 6 Gy reduced the CD34-positive cell population (26.15% CD34-positive cell) compared to the control (41.5% CD34-positive cell) (FIGS. 22A-22D and 23A-23F).

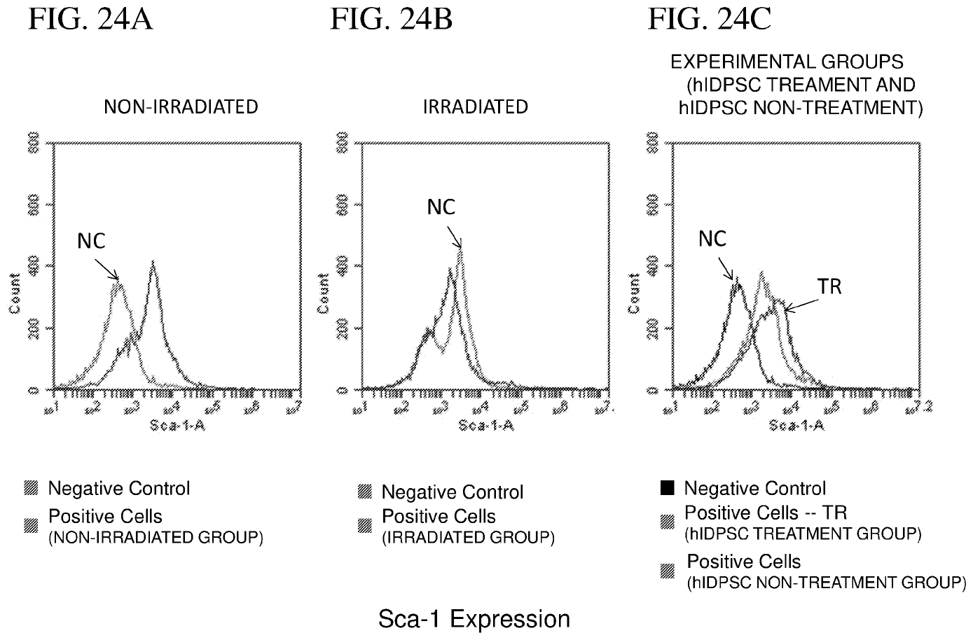

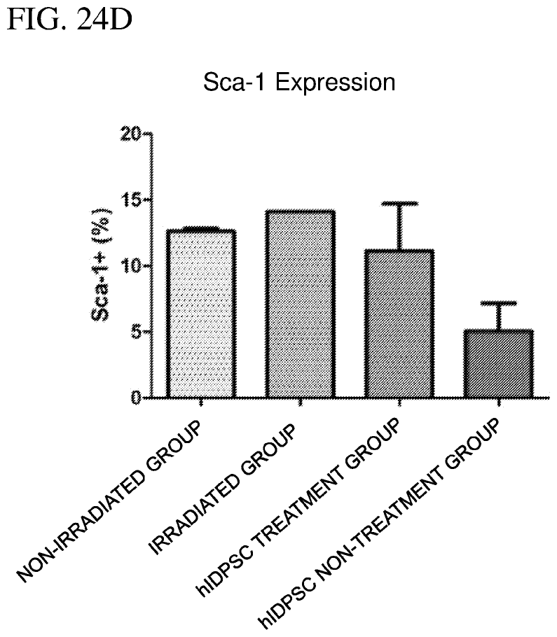

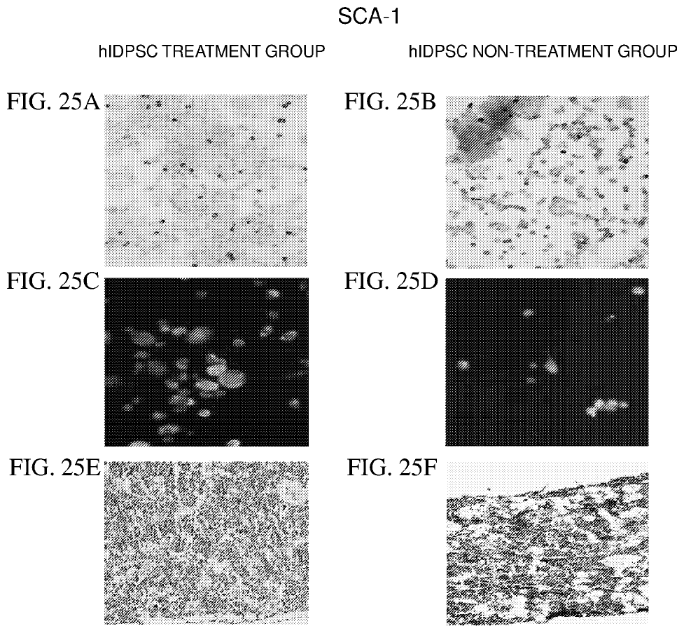

Sca-1 has been shown to play an important in the activation and self-renewal of HSCs. When HSCs are stimulated, they leave their quiescent state and express Sca-1 to promote self-renewal. It has been suggested that radiation causes a homeostatic imbalance of the medullary niche and consequently promotes a strong stimulus of Sca-1 for self-renewal of the remaining HSCs to compensate for other, compromised BM cells. Here, after the irradiation, a slight increase of the Sca-1 was observed (FIGS. 24A-24D and 25A-25F). However, the radiation provided continued oxidative stress, resulting in a decrease of HSCs until the end of the study (D2). This may explain the decrease in Sca-1 expression in the untreated group. However, the IDPSC treated group maintains the expression level of Sca-1 similar to the control (without irradiation), suggesting that IDPSCs can protect HSCs against irradiation, stimulate HSC proliferation, or both.

Reductions of CD34 (26.15% CD34-positive cells) and c-kit (38.5% c-kit-positive cells) were observed after irradiation compared to normal (41.5% CD34-positive cells and 44.26% c-kit-positive cells). However, there was no significant reduction in the percentage of Sca-1-positive cells 48 hours after irradiation (14.1% Sca-1-positive cells) compared to the control (without radiation) (12.6% Sca-1-positive cells) (FIGS. 20, 22, and 24).

Differences in the expression of hematopoietic markers were observed between the IDPSC-treated versus untreated groups. The IDPSC-treated group had a higher percentage of immunopositive cells for markers of HSCs and progenitors (11.3% Sca-1-positive, 33.6% c-kit-positive, and 53.45% CD34-positive) compared to the untreated group (5.06% Sca-1-positive, 28.9% c-kit-positive, and 45.35% CD34-positive) (FIGS. 20, 22, and 24).

The partial recovery of HSCs and their precursors in irradiated mice after IDPSC transplantation suggests that IDPSCs have a role in the stimulation of hematopoiesis.

CD45

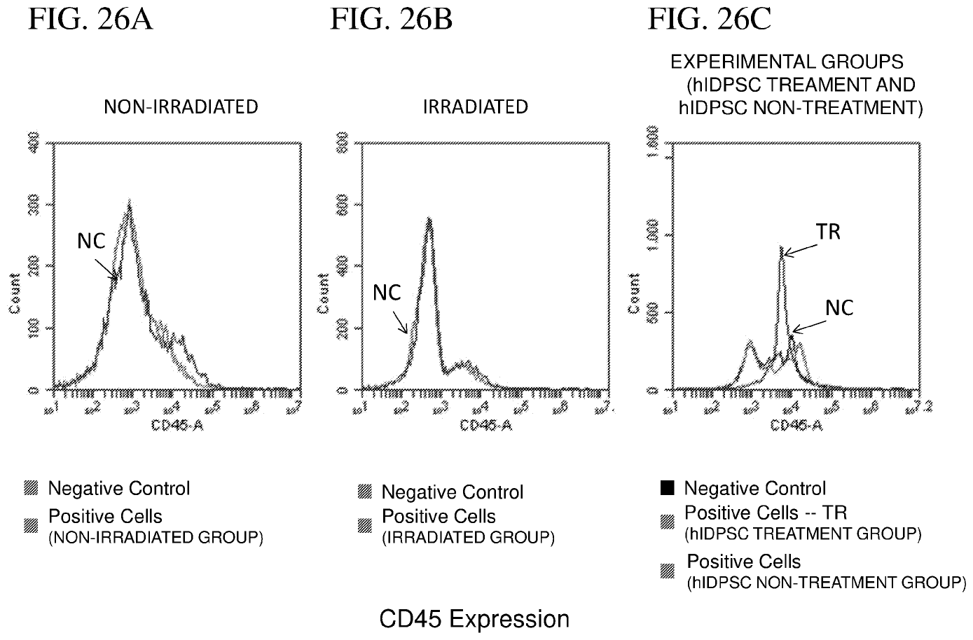

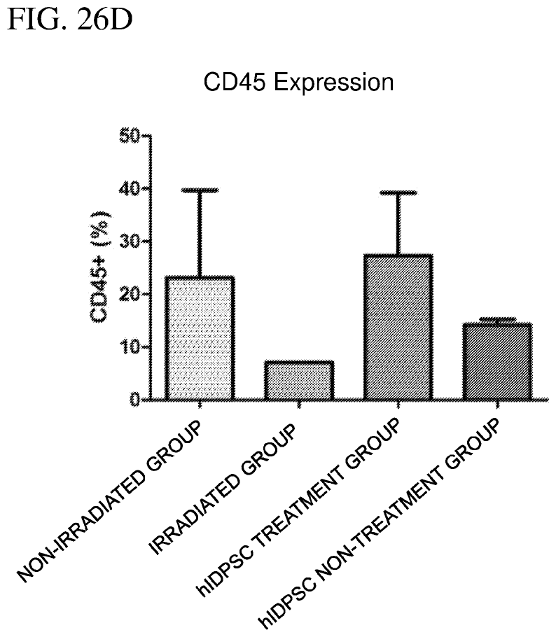

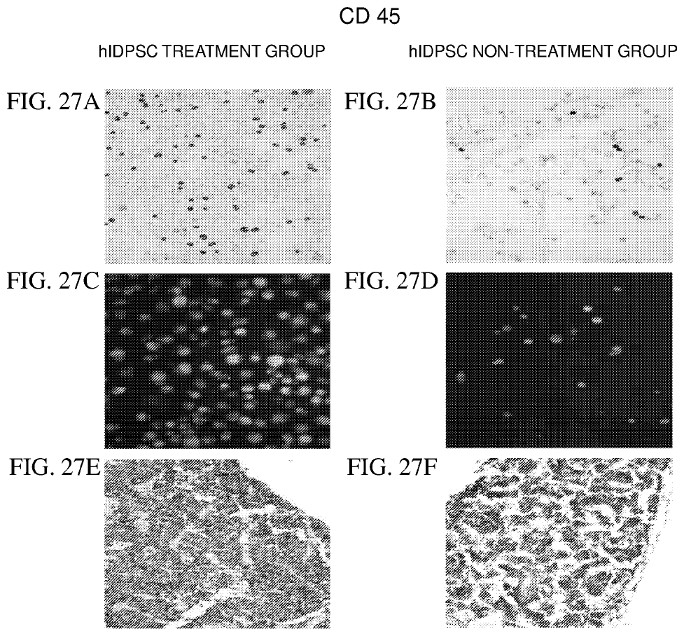

After exposure to radiation, leukocytes are the most affected, making them representative cells for confirming AA. CD45, a cell surface marker of leukocytes, plays an important function in leukocytes, especially in the activation of T lymphocytes. Here, a decrease in CD45 expression was detected after the irradiation (48 hours) (7.1% CD45-positive cells) compared to the control group (23.11% CD45-positive cells) (FIGS. 26A-D). This data confirmed the effectiveness of the experimental model used in this study. Moreover, IDPSC treatment promoted a significant increase in the proportion of CD45-positive lymphocytes and their progenitors (33.5% CD45-positive cells), higher than in the untreated group (14.8% CD45-positive cells) (FIG. 26D). Immunocytofluorescence and immunocytochemical analysis further corroborated our findings. BM aspirate showed a higher number of CD45-positive cells in the treated relative to the untreated group (FIG. 27A-27F).

CD44

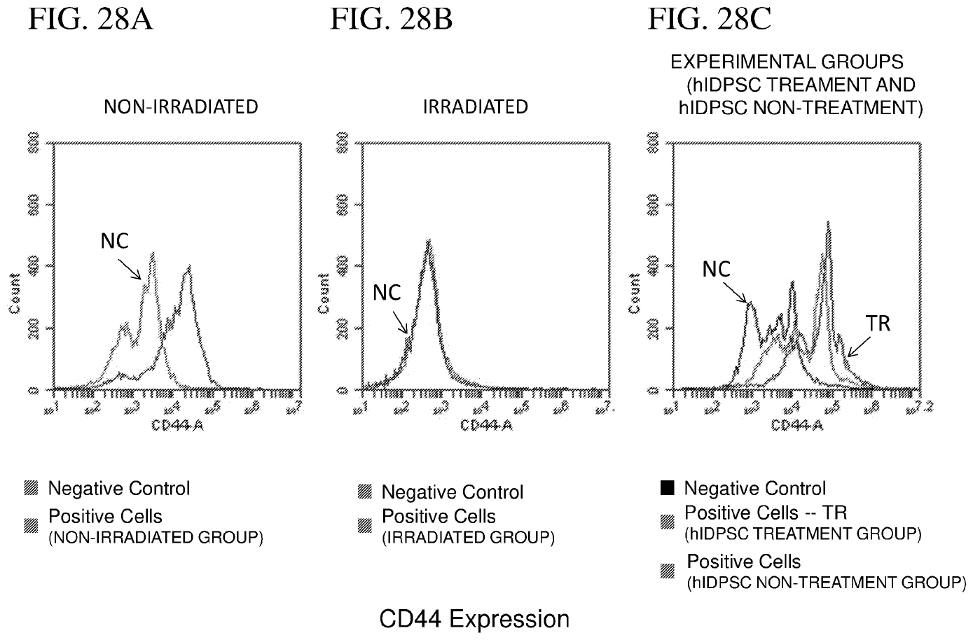

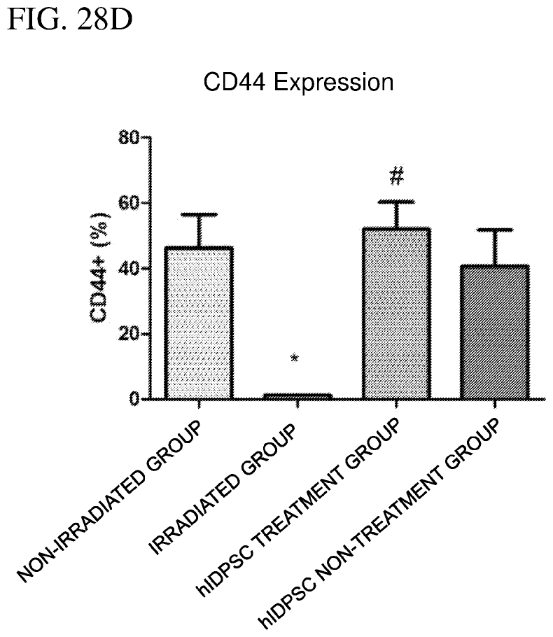

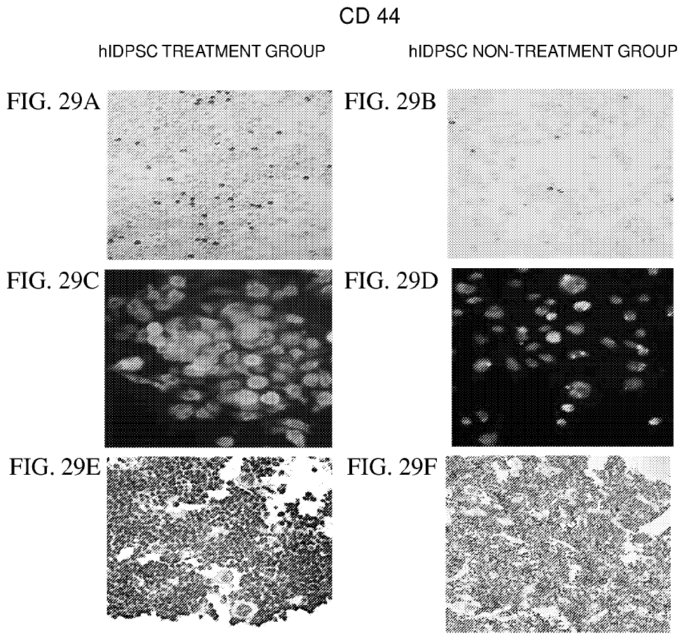

CD44, a cell adhesion molecule, is fundamental in the maintenance of precursor pools and migration of HSCs. CD44 is expressed in lymphocyte, progenitors, and MSC. After irradiation, there was a significant reduction of CD44-positive cells (P<0.05) (mean: 1.2% CD44-positive cells) compared to the control group (46.26% CD44-positive cells). Thus, CD44-positive cells are extremely sensitive to irradiation damages. After IDPSC transplantation, there was a significant recovery of CD44-positive cells in the IDPSCs treated group (P<0.05) (51.9% CD44-positive cells) and untreated group (mean 40.55% CD44-positive cells) (FIGS. 28A-28D and 29A-29F). Finally, medullary aspirate also demonstrated a higher percentage of CD44-positive cells in IDPSC treated group compared to the untreated group (FIG. 28D).

Hematopoiesis is a complex process. Hematopoiesis depends on a set of HSCs and progenitor cells to maintain the quiescent state and protect against cytotoxic insults such as irradiation for adequate production of blood cells throughout life (self-cell renewal). Quiescence and self-renewal are two important characteristics of HSCs. Following the stimulus, HSCs either auto renovate to maintain the cellular pool in their niche or proliferate and differentiate into progenitor cells eventually giving rise to differentiated cells in the peripheral blood. Our results strongly suggest that IDPSCs can provide stimulation to the intrinsic hematopoietic cells.

IDPSC Action in Stromal Niche

CD90

MSCs are naturally present in the medullary stroma and are important in the reconstitution of the medullary microenvironment through the secretion of various growth factors and interleukins such as IL-6, IL-7, IL-8, IL-11, IL-14, IL-15, Flt-3 linker, and stem cell factor. In vivo studies have also demonstrated the potential of MSCs in hematopoiesis. The co-transplantation of MSC with HSCs provides several benefits for successful cellular transplantation for the patients, including facilitating grafting and facilitating recovery of hematopoiesis.

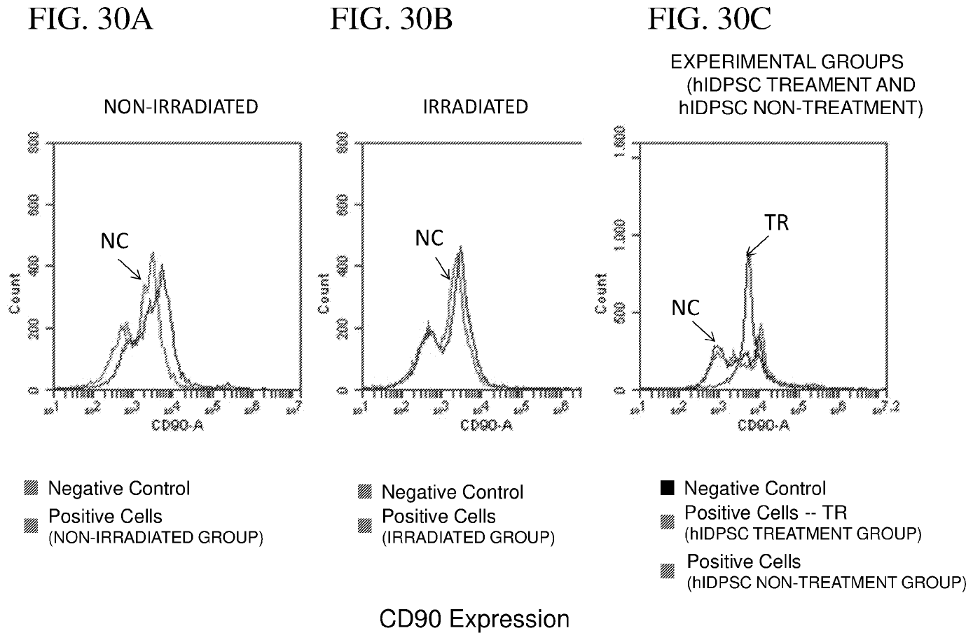

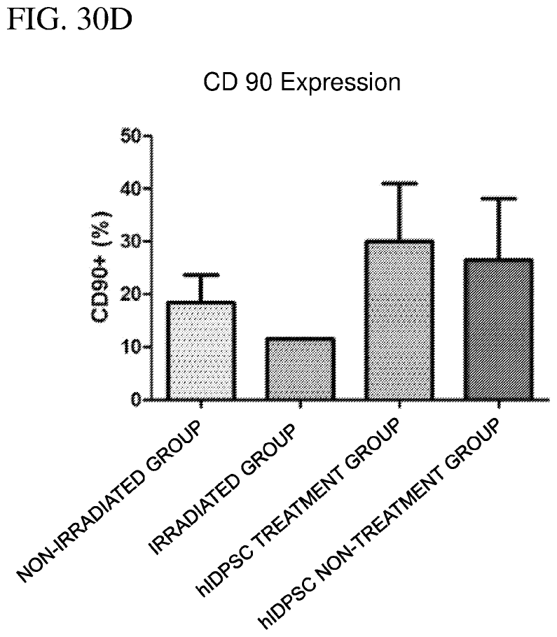

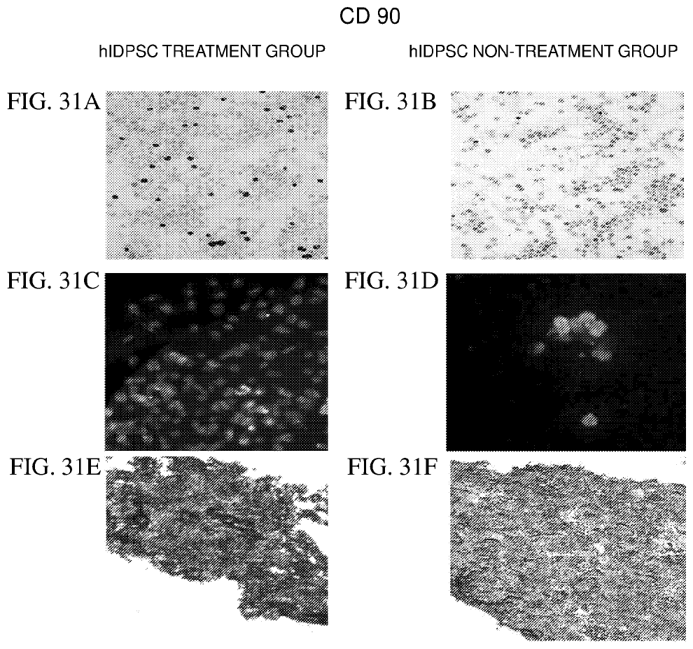

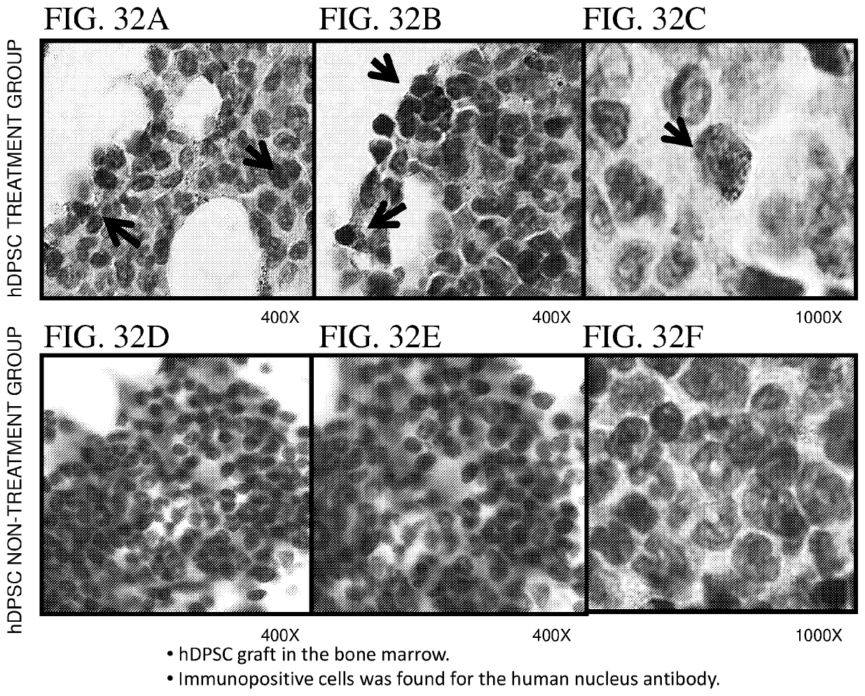

MSCs are heterogeneous cells that express markers such as CD44, C105, and CD90. We used antibodies against CD90 to determine the effects of radiation in MSCs and additional therapeutic benefits of IDPSC transplantation. CD90 is a surface marker highly expressed in MSCs and IDPSCs (CAPLAN et al., 2006; KERKIS et al., 2006). Flow cytometry demonstrated that irradiation caused a decrease in CD90-positive cells (11.5%) compared to the control (18.4%). CD90-positive cells is slightly increased in the IDPSC treated group (29.9%) compared to the untreated group (26.42%) (FIG. 30A-D). Immunocytofluorescence and immunocytochemistry assays corroborate the flow cytometry results, detecting a higher percentage of CD90-positive cells in the group treated with IDPSCs (FIGS. 31A-31F). These results are consistent with an increased expression of CD44 and CD105, which are also MSC markers.

### Example 8. IDPSCs Migrating and Homing in BM

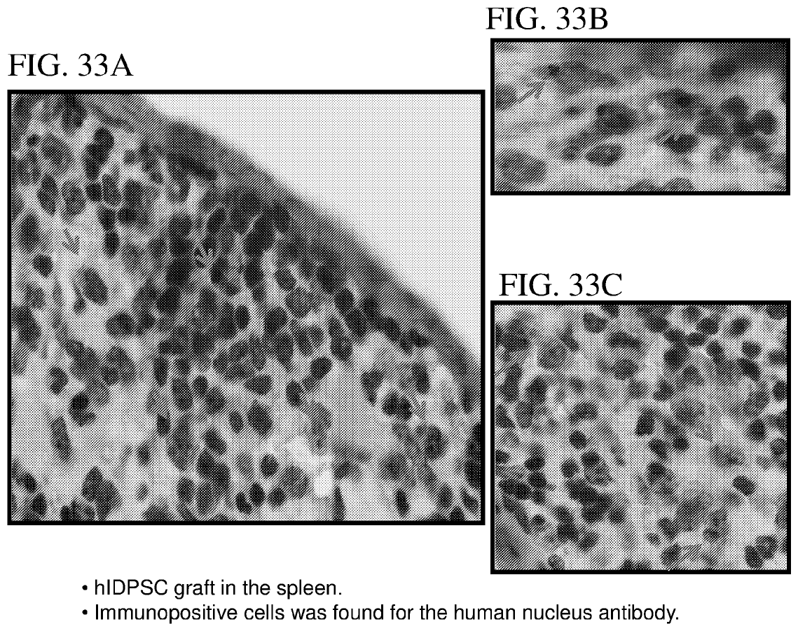

Although IDPSCs were administrated intraperitoneally, they were able to migrate and to home in BM. Using an anti-human nucleus antibody; IDPSCs were identified in BM of murine BM (FIGS. 32A-32C). Homing was not observed in the untreated group (FIGS. 32D-32F). Importantly, to date, there have been no studies to demonstrate that MSCs can migrate to BM in mouse models of AA or acute radiation syndrome. We also observed IDPSC homing in the spleen of the treated group (FIGS. 33 A-33C) but not the untreated group (data not shown).

### Example 9. Long-Term Effects of IDPSC Transplantation in Animal AA Model

To investigate whether the recovery of blood parameters promoted by IDPSC transplant (D62) was reversed, a long-term follow-up (6 months) was performed using hematimetric indexing, flow cytometry, and conventional histology. The data were compared with the same analysis on the animals euthanized at D62.

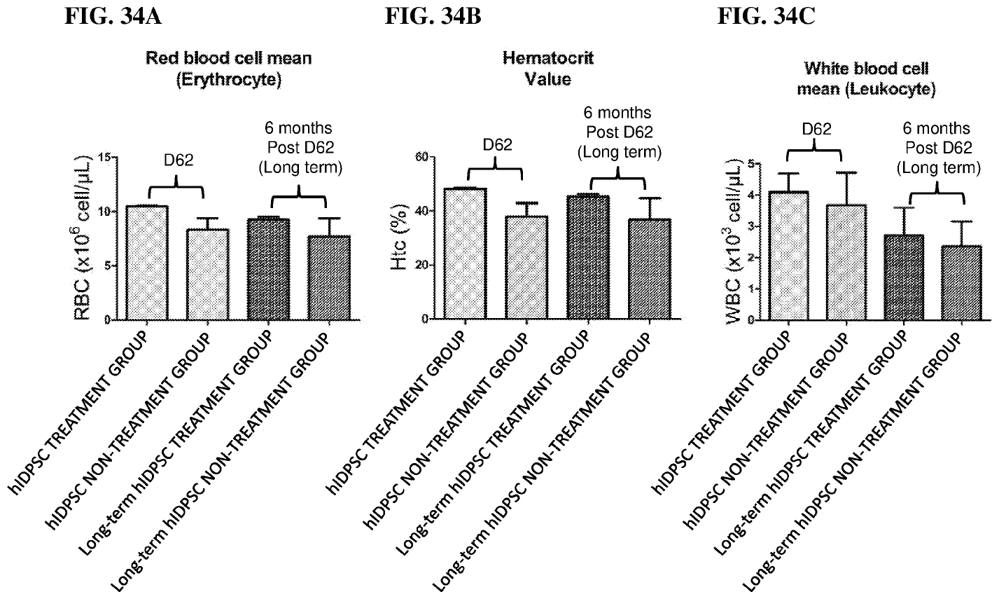

The hematimetric indexes (erythrocytes, hematocrit, and leukocytes) evaluated within 6 months after the third transplant (Post-D62) did not show a significant difference compared to D62 (FIGS. 34A-34C). However, a slight decrease in the blood parameters was observed after 6 months that was not statistically significant. Nevertheless, the result suggests that peripheral blood cells show a tendency of preserving BM recovery archived after IDPSC transplantation.

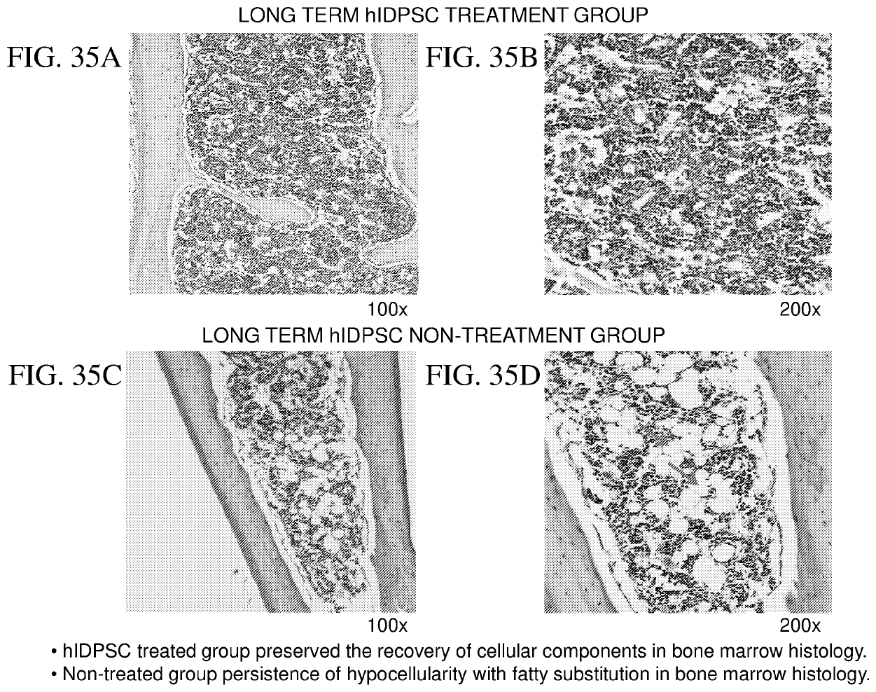

The histology of BM of the long-term experimental group (6 months) was performed, and the result showed that the BM cellularity in IDPSC treated group was preserved (FIGS. 35A-35B). The BM of the untreated group demonstrated the persistence of a hypocellularity with fat substitution (FIGS. 35C-35D) compared to the BM in IDPSC treated group, showing fully filled and high cellularity medullary canal (FIGS. 35A-35B). These data support the regenerative potential of IDPSC in AA.

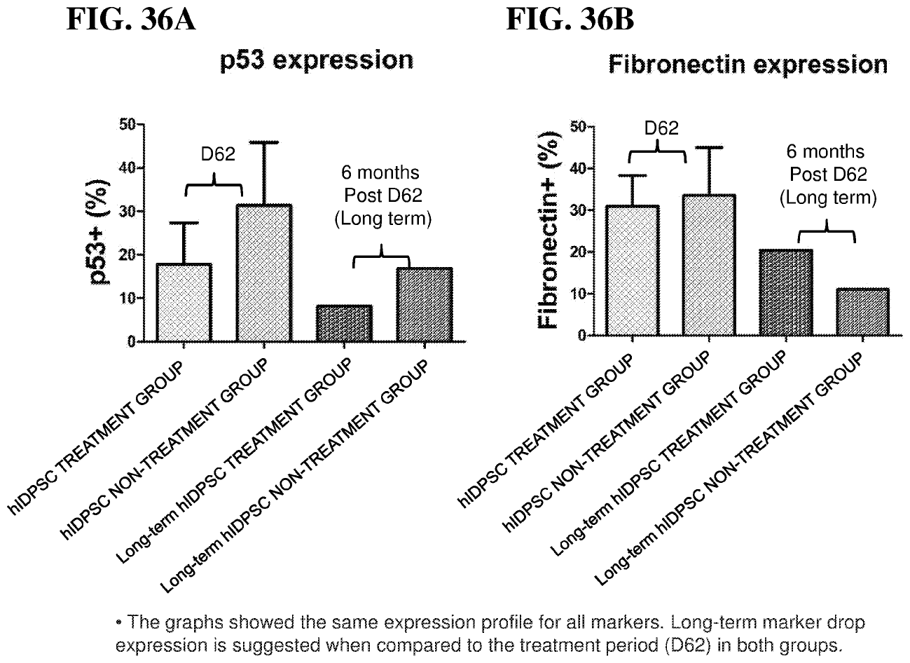

Cell death was also analyzed in this long-term group. The results revealed exposure to irradiation still affects BM (after 5 months), evidenced by the greater expression of p53 in the untreated group (FIG. 36A). In contrast, the expression of p53 was significantly lower in the IDPSC treated group. Thus, IDPSCs can decrease the side effect of cell death caused by radiation even after six months.

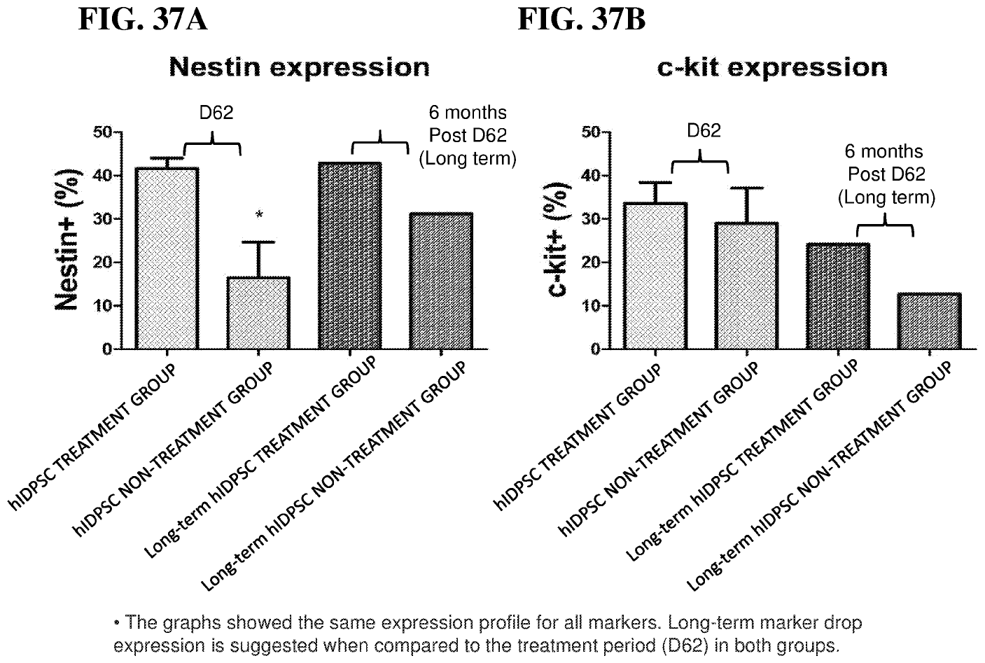

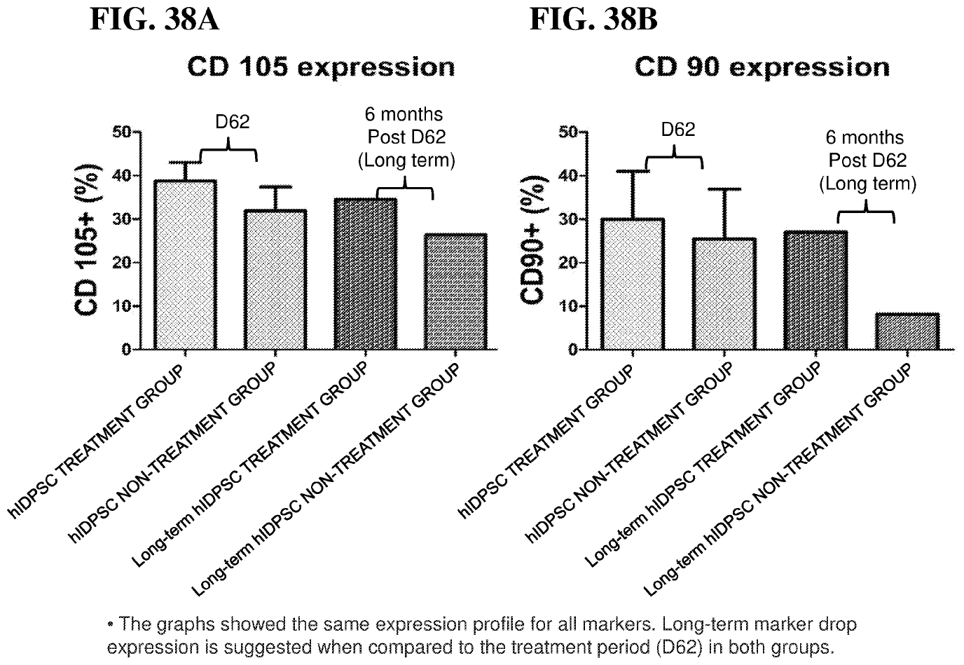

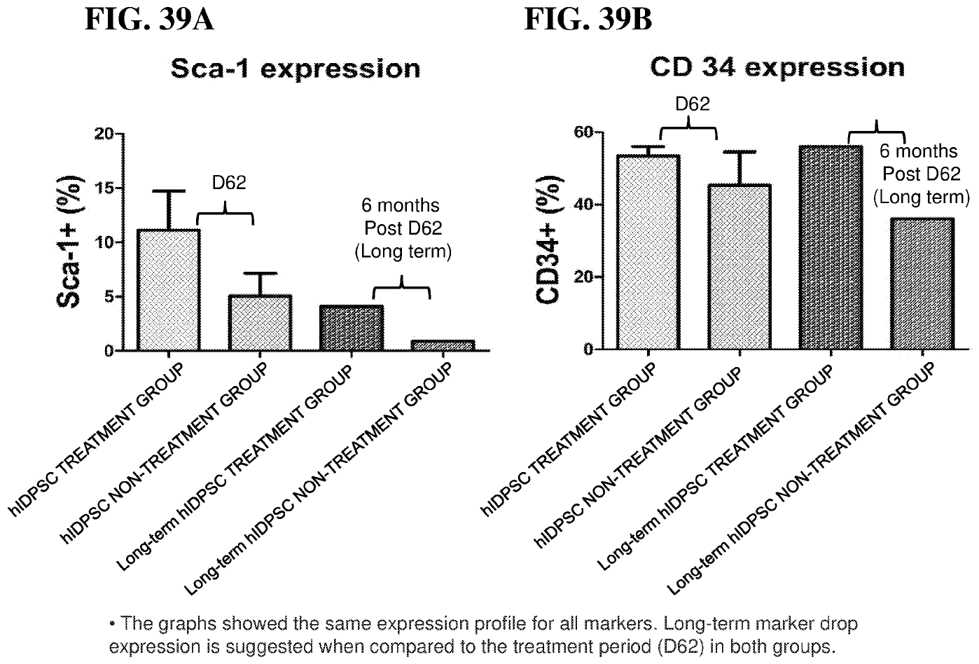

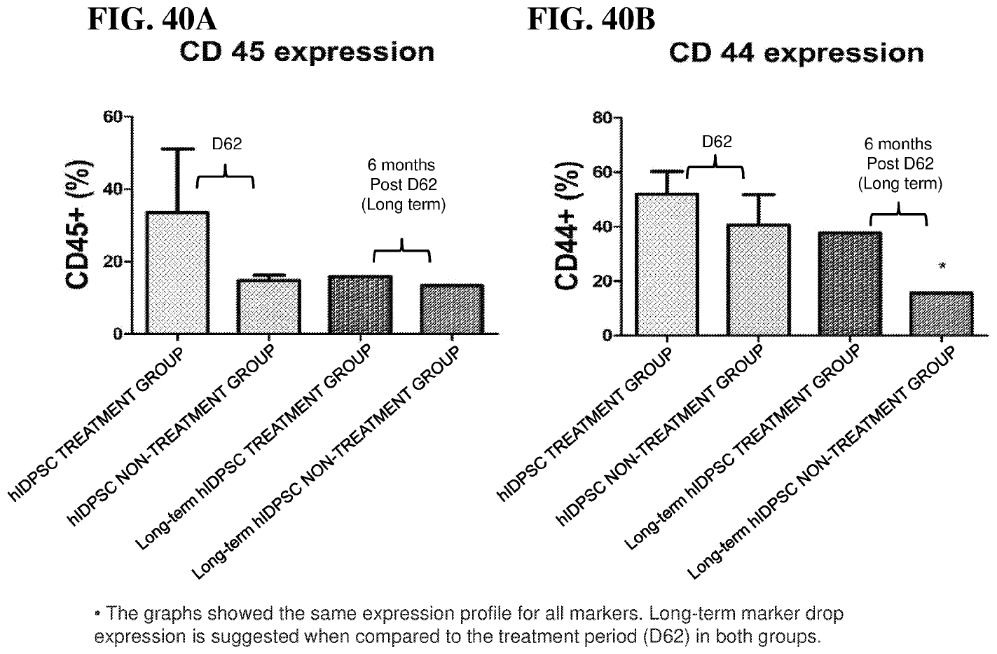

The expression of biomarkers was also analyzed in the long-term group (FIGS. 36B-36C, 37A-37C, and 40A-40C). In general, the expression of stromal and hematopoietic markers decreased slightly over time (6 months) in both experimental groups. However, no significant difference in marker expression was observed between the short-term and the long-term groups. IDPSC transplantation appears to support regeneration of BM intrinsic cells after exposure to 6 Gy irradiation.

### Example 10. Benefits of IDPSC Transplantation on Side Effects of Radiation Therapy

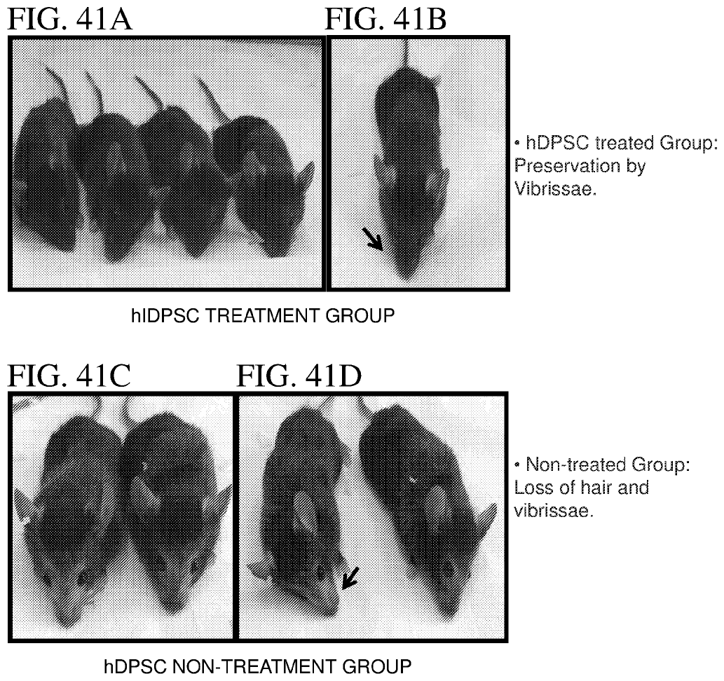

Ionic radiation affects an entire living organism. Acute reactions to radiation cause oxidative stress and inflammation that alter the microenvironment and injure normal tissues. Hair loss is one of the first noticeable damages. Here, the IDPSC treated group did not present loss hair and vibrissae until the end of the study (D62) (FIGS. 41A-41B). In contrast, the untreated group showed significant loss of hair and the vibrissae after irradiation (FIGS. 41C-41D).

Previous studies have shown that whole-body radiation at the dose of 6 Gy causes skin damages (around 2%), and that transplanting MSCs directly into the injured area rapidly regenerates the radiation-induced lesion. Radiation-induced hair loss is caused by irreversible defects in the self-renewal and/or development of follicular melanocyte stem cells in the hair follicles. Our results demonstrate hair preservation in animals, which received ionic radiation and IDPSC transplantation. This was not observed in animals that not received IDPSC therapy. These data further suggest IDPSCs' protective effects against ionic irradiation damage. In contrast to previous studies, which demonstrated local effect after topic application of MSC, the present invention provides generalized protective effects with systemic IDPSC infusion.

### Example 11. Dosage of Blood Plasma Cytokines by CBA—Cytometric Bead Array

The cytokine dosage was performed from the blood plasma of the animals, previously stored in a freezer at −80° C. A new kit, the murine CBA (Th1/Th2/Th17) provided by BD®, was used to perform the cytokine plasma levels: Interleukin-2 (IL-2), Interleukin-4 (IL4), Interleukin-6 (IL-6), Interferon-γ (IFN-γ), Tumor Necrosis Factor Alpha (TNF-α), Interleukin-17A (IL-17A). The samples were read by the BD® Canto II Flow Cytometer provided by the Immunochemistry Laboratory of the Butantan Institute, and the result obtained was analyzed by FCAP Array Software v3.0 software. After the experiments were carried out, the two groups were compared using two-way statistical analysis (ANOVA) using GraphPad Prism 5.0 software (GRAPHPAD; PRISM, 2007). We will consider those P values lower than 0.05, significant.

Tumor necrosis factor (TNF-α) is a pleiotropic cytokine known for its tumor cytotoxicity, besides being a potent inflammatory mediator and regulator of homeostatic functions and antimicrobial immunity. It is produced by activated T lymphocytes and activated macrophages. TNF-α is a proinflammatory cytokine capable of acting in its soluble or cell membrane-associated form. The TNF-α cytokine has been shown to be an important regulator of cell proliferation, survival, differentiation and apoptosis, through two transmembrane receptors, TNFR1 and TNFR2.

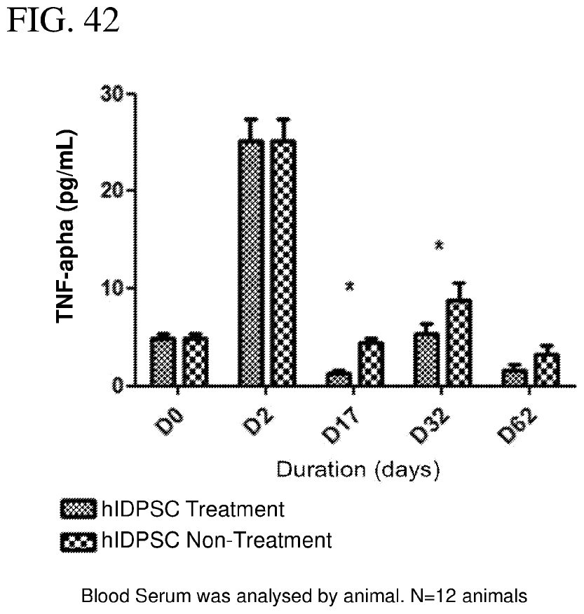

The data presented by the graph (FIG. 42) suggest that the greater participation of the cytokine TNF-α occurs in the first hours after the stimulation (D2), where its transcription causes the release of several factors that promote the acute inflammation in the animal. Although levels of TNF-α in untreated animals tend to decrease throughout the assay. As well as presented in the work done by DE AGUIAR et al., 2018, cellular therapy with IDPSC promotes the reduction of TNF-α levels in animals more efficiently and rapidly compared to those that did not receive treatment. Thus, it is possible to suggest that cellular therapy with IDPSC promoted the reduction of serum levels of TNF-α, thus promoting the attenuation of inflammation and preserving the homeostasis of the animal.

Interleukin-4 is a multifunctional cytokine that participates in the regulation of the immune system at several levels. Known as a growth factor and lymphocyte survival, the cytokine IL-4 also acts as a differentiation factor and stimulation of B lymphocytes. It is produced from the stimulation of CD4+ and CD8+ T cell receptors, in addition to basophils and mast cells. It has been shown that IL-4 has an important anti-inflammatory role, acting on leukocyte survival and tissue repair, activating the “alternative” pathway of macrophages. In addition, IL-4 is able to induce inhibition of the proinflammatory cytokine TNF-α through the transcription factor STATE.

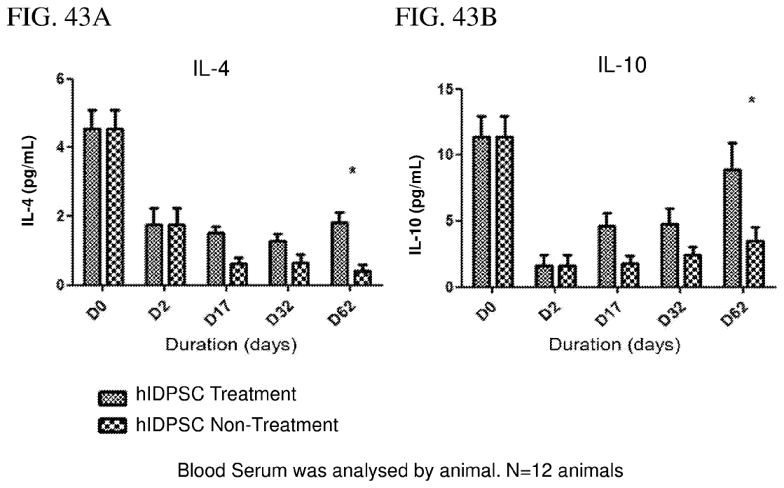

The data on the graph (FIG. 43A) show that after irradiation (D2) there was a large decrease in IL-4 values in relation to D0 due to the increase in inflammation caused by ionic radiation. During the three IDPSC therapies (D17, D32 and D62) it is possible to observe that IL-4 levels in the IDPSC-treated group are higher in relation to the untreated group, which present only a constant decrease in IL-4. In D62, the group receiving IDPSC treatment showed a significant increase compared to the group without treatment.

Thus, it is possible to suggest that IDPSC treatment may have promoted anti-inflammatory action by increasing the secreted levels of IL-4 in the animals that received IDPSC transplantation. In addition, it is also possible to suggest that the increase of IL-4 may have led to the decrease of the cytokine TNF-α, since this result corroborates with that also found in TNF-α. This suggests another benefit of cellular therapy with hIDPSC.

Interleukin-10 is a cytokine with strong anti-inflammatory properties, protecting the body from inflammation damage and reestablishing homeostasis. Produced by a variety of cells, IL-10 can be secreted in the acute and/or chronic phase of inflammation. The IL-10 cytokine acts by limiting responses of Th1 and Th2 profile cells, thereby inhibiting the secretion of proinflammatory cytokines and chemokines.

The data presented in the graph show (FIG. 43B) that after irradiation (D2) IL-10 levels fell sharply compared to the period before irradiation (D0). However, after the first therapy (D17) levels of IL-10 only in the group receiving cell therapy with IDPSC increased relative to D2.

Throughout the treatment period (D17, D32 and D62) the IDPSC treated group had the highest level of IL-10 compared to the untreated group. One month after the third therapy (D62), the level of IL-10 in the group receiving IDPSC increased so much that it showed a significant increase (*) compared to the group that did not receive the treatment, even reaching value close to that found before irradiation (D0).

Because IL-10 is a cytokine with potent anti-inflammatory properties, it is possible to suggest that cellular therapy treatment with IDPSC may have stimulated IL-10 cytokine secretion and consequently modulated inflammation and decreased prokaryotic cytokine secretion inflammatory.

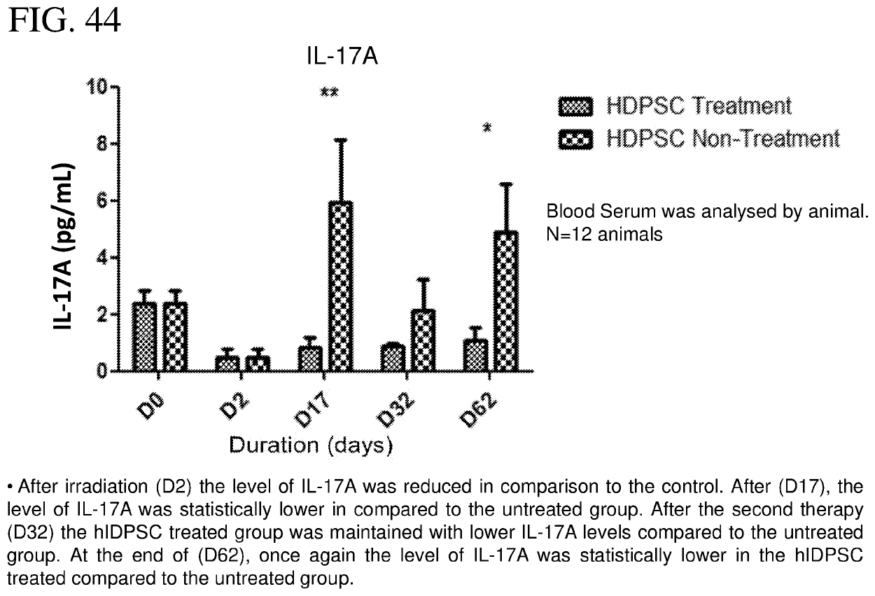

Interleucina-17A is predominantly secreted by cells of Th17 profile, the cytokine IL-17A is important in the development of inflammatory diseases, being characterized, therefore, pro-inflammatory. IL-17A expression is increased in autoimmune disease. The data presented in the graph (FIG. 44) show that after irradiation (D2), IL-17A levels were reduced in relation to D0. Already after the first therapy with CTIPDh (D17), IL-17A levels in the group that did not receive treatment showed a significant increase in relation to the treated group. After the second therapy (D32), IL-17A levels remained lower in the IDPSC-treated group compared to the non-treated group. Finally, in D62, the treated group was shown to have stabilized and low levels, while the group that did not receive treatment again showed a significant increase in relation to the treatment.

Thus, it is possible to suggest that IL-17A presented late response, thus demonstrating a possible role in chronic inflammation. Besides that, IDPSC cell therapy has been shown to stabilize IL-17A levels, preventing the progression of inflammation in treated animals. While the absence of the treatment only promoted the continuation of the increase of the inflammation.

The data presented were important since they suggest that the IDPSC cell, a type of MSC that possesses anti-inflammatory properties, and can thus promote the modulation of inflammation.

### REFERENCES

1. Caplan & Dennis, Mesenchymal Stem Cells as Trophic Mediators, 98 J. Cell Biochem. 5, 1076-84 (2006).
2. Hu et al., The Radiation Protection and Therapy Effects of Mesenchymal Stem Cells in Mice with Acute Radiation Injury, 83 Br. J. Radiol. 985, 52-8 (2010).
3. Ivanova et al., Dissecting Self-Renewal in Stem Cells with RNA Interference, 442 Nature 7102, 533-38 (2006).
4. Kerkis et al., Isolation and Characterization of a Population of Immature Dental Pulp Stem Cells expressing OCT-4 & Other Embryonic Stem Cell Markers, 184 Cells Tissues Organs. 3-4, 105-16 (2006).
5. Lizier et al., Scaling-Up of Dental Pulp Stem Cells Isolated from Multiple Niches, 7 PLoS ONE 6, e39885 (2012).
6. Sacchetti et al., No Identical “Mesenchymal Stem Cells” at Different Times and Sites: Human Committed Progenitors of Distinct Origin and Differentiation Potential Are Incorporated as Adventitial Cells in Microvessels, 6 Stem Cell Reports 6, 897-913 (2016).
7. Takahashi & Yamanaka, Induction of Pluripotent Stem Cells from Mouse Embryonic and Adult Fibroblast Cultures by Defined Factors, 126 Cell 4, 663-76 (2006).
8. Weiss & Wardrop, Schalm's Veterinary Hematology, WILEY-BLACKWELL (2011).
9. U.S. Pat. No. 9,790,468
10. PCT/US2013/047435
11. U.S. Pat. No. 8,545,833 B2
12. US20130273011 A1
13. WO2010033285 A2
14. WO2012127320 A1
15. WO2013012698 A1

1. A method of treating or preventing bone marrow failure, comprising administering to a subject a composition comprising a population of isolated immature dental pulp stem cells (IDPSCs), wherein the IDPSCs are in an amount sufficient to treat or prevent bone marrow failure in the subject.

2. The method of claim 1, wherein the administered IDPSCs are capable of migrating to the bone marrow of the subject.

3. The method of claim 1, wherein the bone marrow failure is caused by a radiation exposure at a dose of 1-30 Gy.

4. The method of claim 3, wherein the composition is administered between 24 and 60 hours after the radiation exposure.

5. The method of claim 1, wherein the IDPSCs are in an amount sufficient to increase at least one of the following:
fibroblast precursors in the bone marrow of the subject,
erythrocyte (red blood cell) count in the bone marrow of the subject;
hematocrit level in the bone marrow of the subject;
leukocyte (white blood cell) count in the bone marrow of the subject; and
megakaryocytes, Sca-1-positive cells, Nestin-positive cells, fibronectin-positive cells, CD44-positive cells, or a combination thereof in the bone marrow of the subject.

6. (canceled)

7. (canceled)

8. (canceled)

9. (canceled)

10. The method of claim 1, wherein the IDPSCs are in an amount sufficient to cause at least one of the following: an increase in bone marrow cellularity in the subject; an increase in extracellular matrix remodeling in the bone marrow of the subject a reduction of bone marrow cell apoptosis in the subject.

11-12. (canceled)

13. The method of claim 1, wherein the IDPSCs are in an amount sufficient to reduce serum levels of TNF-α, IL-17A, or both in the subject; and/or to increase serum levels of IL-4 and IL-10 in the subject.

14. (canceled)

15. The method of claim 1, wherein the IDPSCs are in an amount sufficient to improve hematopoietic recovery for at least 6 months.

16. The method of claim 1, wherein the composition is administered at least twice at an interval of between 10 and 20 days.

17. A composition for treating bone marrow failure, comprising a population of isolated immature dental pulp stem cells (IDPSCs) prepared by obtaining a dental pulp (DP); establishing an explant culture by placing the DP onto a plastic surface in a culture medium and allowing IDPSCs to migrate out of the DP; passaging the IDPSCs to expand the explant culture; mechanically transferring the DP onto a different plastic surface in the culture medium; and collecting the IDPSCs that adhere to the plastic surface from the explant culture, the population of IDPSCs comprising IDPSCs, wherein the IDPSCs are obtained after five transfers and four or five passages.

18. The method of claim 1, wherein at least 80% of the IDPSCs express CD117 (c-kit), Fibronectin, or both.

19. The method of claim 1, wherein 96%-99% of the IDPSCs are positive for at least one of the following: CD105, CD73, CD90, CD44, CD117, Nestin, Vimentin, Fibronectin, or combinations thereof; and/or 96%-99% of the IDPSCs are negative for at least one of the following: CD45, CD34, HLA DR cells, or combinations thereof.

20-27. (canceled)

28. The method of claim 1, wherein the composition further comprises bone-marrow stem-cells, hematopoietic stem cells, or both.

29. The method of claim 1, wherein the subject is human and the IDPSCs are allogeneic.

30. The method of claim 1, wherein the composition comprises 5×10⁶-1×10⁹ IDPSCs.

31. The composition of claim 17, wherein at least 80% of the IDPSCs express CD117, Fibronectin, or both.

32. The composition of claim 17, wherein 96%-99% of the IDPSCs are positive for at least one of the following: CD105, CD73, CD90, CD44, CD117, Nestin, Vimentin, Fibronectin, or combinations thereof; and/or 96%-99% of the IDPSCs are negative for at least one of the following: CD45, CD34, HLA DR cells, or combinations thereof.

33. The composition of claim 17, wherein the composition further comprises bone-marrow stem-cells, hematopoietic stem cells, or both.

34. The composition of claim 17, wherein the subject is human and the IDPSCs are allogeneic.

35. The composition of claim 17, wherein the composition comprises 5×10⁶-1×10⁹ IDPSCs.
